SEQUENCE LISTING (8 sequences)

SEQ ID NO: 1 - DNA, 50 bp, Artificial
  Primer sequence
gcttggcgcgcactccgaggtccaactggtggagagcggtggaggtgtta

SEQ ID NO: 2 - DNA, 60 bp, Artificial
  Primer sequence
gcgacgtcgacaggactcacctgaggagacggtgaccggggtcccttggccccagtaagc

SEQ ID NO: 3 - DNA, 50 bp, Artificial
  Primer sequence
gcttggcgcgcactccgacatccagatgacccagagcccaagcagcctga

SEQ ID NO: 4 - DNA, 60 bp, Artificial
  Primer sequence
gcccttaattaagttattctactcacgtgtgatttgcagcttggtcccttggccgaacgt

SEQ ID NO: 5 - DNA, 1380 bp, Human/murine chimera
  CDS (1)..(1380)
gaggtccaactggtggagagcggtggaggtgttgtgcaacctggccggtccctgcgcctgtcctgctccacgtctggcttcactttcagtgactattacatgtattgggtgagacaggcacctggaaaaggtcttgagtgggttgcatacatgagtaatgttggtgctatcaccgactatccagacactgtgaaggggagatttacaatatcgagagacaacagcaagaacacattgttcctgcaaatggacagcctgagacccgaagacaccggggtctatttttgtgcaagaggcacccgtgatggctcgtggtttgcttactggggccaagggaccccggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagccctgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatgagtgcgacggccgtctagacccggggaattc

SEQ ID NO: 6 - protein, 449 aa, Human/murine chimera
  Human/Murine Chimera G193 Heavy Chain
EVQLVESGGGVVQPGRSLRLSCSTSGFTFSDYYMYWVRQAPGKGLEWVAYMSNVGAITDYPDTVKGRFTISRDNSKNTLFLQMDSLRPEDTGVYFCARGTRDGSWFAYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEALGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - DNA, 657 bp, Human/murine chimera
  CDS (1)..(657)
gacatccagatgacccagagcccaagcagcctgagcgccagcgtgggtgacagagtgaccatcacctgtagatctagtcagcgcattgtacatagtaatggaaacacctatttagaatggtaccagcagacgccaggtaaggctccaaagctgctgatctacaaagtttccaaccgattttctggtgtgccaagcagattcagcggtagcggtagcggtaccgacttcaccttcaccatcagcagcctccagccagaggacatcgccacctactactgctttcaaggttcacatgttcccttcacgttcggccaagggaccaagctgcaaatcacacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 8 - protein, 219 aa, Human/murine chimera
DIQMTQSPSSLSASVGDRVTITCRSSQRIVHSNGNTYLEWYQQTPGKAPKLLIYKVSNRFSGVPSRFSGSGSGTDFTFTISSLQPEDIATYYCFQGSHVPFTFGQGTKLQITRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2006/0002942 A1
(43) Pub. Date: Jan. 5, 2006

(54) Calicheamicin conjugates

(75) Inventors: Arthur Kunz, New City, NY (US); Justin Keith Moran, Valley Cottage, NY (US); Joseph Thomas Rubino, Towaco, NJ (US); Neera Jain, Monroe, NY (US); Erwin Raymond Arsene Boghaert, Monroe, NY (US); Philip Ross Hamann, Thiells, NY (US); Mark Edward Ruppen, Garnerville, NY (US); Nitin Krishnaji Damle, Upper Saddle River, NJ (US); Eugene Vidunas, Middletown, NY (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 11/080,587

(22) Filed: Mar. 15, 2005

Related U.S. Application Data
(60) Provisional application No. 60/553,112, filed on Mar. 15, 2004.

(51) Int. Cl. A61K 39/395; C07K 16/46

(52) U.S. Cl. 424/178.1; 530/391.1

(57) ABSTRACT

Anti-Lewis Y antibodies are described. Methods for preparing monomeric cytotoxic drug/carrier conjugates with a drug loading significantly higher than in previously reported procedures and with decreased aggregation and low conjugate fraction (LCF) are described. Cytotoxic drug derivative/antibody conjugates, compositions comprising the conjugates and uses of the conjugates are also described. Specifically, monomeric calicheamicin derivative/anti-Lewis Y antibody conjugates, compositions comprising the conjugates and uses of the conjugates are also described.

This application claims priority from copending provisional application Ser. No. 60/553,112 filed on Mar. 15, 2004 the entire disclosure of which is hereby incorporated by reference.

### FIELD OF THE INVENTION

The present invention relates to methods for the production of monomeric calicheamicin cytotoxic drug conjugated to an IgG1 antibody having higher drug loading and substantially reduced low conjugate fraction (LCF). Particularly, the invention relates anti-Lewis Y antibody conjugated to calicheamicin. The invention also relates to the uses of these conjugates.

### BACKGROUND OF THE INVENTION

The use of cytotoxic chemotherapy has improved the survival of patients suffering from various types of cancers. Used against select neoplastic diseases such as, e.g., acute lymphocytic leukemia in young people (Kalwinsky, D. K. (1991) 3: 39-43, 1991) and Hodgkin lymphomas (Dusenbery, K. E, et al. (1988) American Journal of Hematology, 28: 246-251), cocktails of cytotoxic drugs can induce complete cures. Unfortunately, chemotherapy, as currently applied, does not result in complete remissions in a majority of cancers. Multiple reasons can explain this relative lack of efficacy (for review see: Gottesman, M. M. (2002) Ann. Rev. of Med. 53, 615-62; Mashima, T. et al. (1998) Biotherapy: 12(6), 947-952; Mareel, M. M. et al. (1986) Radiotherapy and Oncology: 6, 135-142. Among these, the low therapeutic index of most chemotherapeutics is a likely target for pharmaceutical improvement. The low therapeutic index reflects the narrow margin between the efficacious and toxic dose of a drug, which may prevent the administration of sufficiently high doses necessary to eradicate a tumor and obtain a curative effect.

One strategy to circumvent this problem is the use of a so-called magic bullet. The magic bullet was conceived by Ehrlich (Ehrlich, P. (Collected Studies on Immunity 2, 442-447) and consists of a cytotoxic compound that is chemically linked to an antibody. Binding a cytotoxic anticancer drug to an antibody that recognizes a tumor-associated-antigen can improve the therapeutic index of the drug. This antibody should ideally recognize a tumor-associated antigen (TAA) that is exclusively expressed at the surface of tumor cells. This strategy allows the delivery of the cytotoxic agent to the tumor site while minimizing the exposure of normal tissues. The antibody can deliver the cytotoxic agent specifically to the tumor and thereby reduce systemic toxicity.

Drug conjugates developed for systemic pharmacotherapy are target-specific cytotoxic agents. The concept involves coupling a therapeutic agent to a carrier molecule with specificity for a defined target cell population. Antibodies with high affinity for antigens are a natural choice as targeting moieties. One such antigen is the Lewis Y antigen, which is expressed in normal tissues, but the level of expression is higher in certain tumor types. The Lewis Y (Le^{y}) antigen is found on cells of some breast, colon, gastric, esophageal, pancreatic, duodenal, lung, bladder and renal carcinomas and gastric and islet cell neuroendrocrine tumors. Its presence on some tumor cells is not accompanied by an increase in its serum levels, thus administered Lewis Y specific antibody is not significantly bound by soluble antigen.

With the availability of high affinity monoclonal antibodies, the prospects of antibody-targeting therapeutics have become promising. Toxic substances that have been conjugated to monoclonal antibodies include toxins, low-molecular-weight cytotoxic drugs, biological response modifiers, and radionuclides. Antibody-toxin conjugates are frequently termed immunotoxins, whereas immunoconjugates consisting of antibodies and low-molecular-weight drugs such as methotrexate and Adriamycin are called chemoimmunoconjugates. Immunomodulators contain biological response modifiers that are known to have regulatory functions, such as lymphokines, growth factors, and complement-activating cobra venom factor (CVF). Radioimmunoconjugates consist of radioactive isotopes, which may be used as therapeutics to kill cells by their radiation or used for imaging. Antibody-mediated specific delivery of cytotoxic drugs to tumor cells is expected to not only augment their anti-tumor efficacy, but also to prevent nontargeted uptake by normal tissues, thus increasing their therapeutic indices.

Immunoconjugates using a member of the potent family of antibacterial and antitumor agents, known collectively as the calicheamicins or the LL-E33288 complex, were developed for use in the treatment of cancers. The most potent of the calicheamicins is designated γ₁^{l}, which is herein referenced simply as gamma. These compounds contain a methyltrisulfide that can be reacted with appropriate thiols to form disulfides, at the same time introducing a functional group such as a hydrazide or other functional group that is useful in attaching a calicheamicin derivative to a carrier. The calicheamicins contain an enediyne warhead (FIG. 1) that is activated by reduction of the —S—S— bond causing breaks in double-stranded DNA.

MYLOTARG® (Sievers, E. L. et al (1999) Blood: 93, 3678-3684), also referred to as CMA-676 or CMA, is the only commercially available drug that works according to this principle. MYLOTARG® (gemtuzumab ozogamicin) is currently approved for the treatment of acute myeloid leukemia in elderly patients. The drug consists of an antibody against CD33 that is bound to calicheamicin by means of an acid-hydrolyzable linker. The disulfide analog of the semi-synthetic N-acetyl gamma calicheamicin was used for conjugation (U.S. Pat. Nos. 5,606,040 5,770,710). This molecule, N-acetyl gamma calicheamicin dimethyl hydrazide, is hereafter abbreviated as CM.

Several lines of experimental evidence reinforce the idea that using antibodies that recognize TAAs different from CD33 could expand the application range of the magic bullet approach. Multiple conjugates of antibodies and chemotherapeutic agents (immunoconjugates) have a proven ability to cure a host of xenografted tumors. Some examples of targeted TAAs are: HER2/neu (Starling, J. J, et al. (1992) Bioconjugate Chemistry: 3(4), 315-322; DiJoseph, J. F et al. (2002) European Journal of Cancer: 38 (suppl. 7), S150); PSCA (Sjogren, H. O, et al. (1997) Cancer Res.: 57, 4530-4536); mucine type glycoproteins (MIRACL-26457) (Zhang, S. et al. (1997) Int. J. Cancer, 73: 50-56; Wahl, A. F. et al (2000) Int. J. Cancer, 93: 590-600); EGFR (Furokawa, K., et al. (1990) Mol. Immunol., 27: 723-732); CEA (Kitamura, K., et al, (1994) Proc. Natl. Acad. Sci. USA., 91: 12957-12961); CD22 (Clarke, K., et al. (2000) Cancer Res., 60: 4804-4811) and Lewis^{y}-antigen (Le^{y}) (Morgan, A., et al (1995) Immunology, 86: 319-324). To achieve a cytotoxic effect, antibodies against these surface antigens were conjugated to pseudomonas exotoxin (DiJoseph, J. F et al., S150), maytansinoid (Sjogren, H. O, et al. 4530-4536; Zhang, S. et al. 50-56), calicheamicin (Wahl, A. F. et al. 590-600; Clarke, K., et al. 4804-4811), RNase (Furokawa, K., et al. 723-732), vinca alkaloid (Kitamura, K., et al., 12957-12961) or doxorubicin (Morgan, A., et al. 319-324).

The use of the monomeric calicheamicin derivative/carrier conjugates in developing therapies for a wide variety of cancers has been limited both by the availability of specific targeting agents (carriers), as well as the conjugation methodologies which result in the formation of protein aggregates when the amount of the calicheamicin derivative that is conjugated to the carrier (i.e., the drug loading) is increased. Since higher drug loading increases the inherent potency of the conjugate, it is desirable to have as much drug loaded on the carrier as is consistent with retaining the affinity of the carrier protein.

The natural hydrophobic nature of many cytotoxic drugs, including the calicheamicins, creates difficulties in the preparation of monomeric drug conjugates with good drug loadings and reasonable yields. The increased hydrophobicity of the linkage, as well as the increased covalent distance separating the therapeutic agent from the carrier (antibody), appears to exacerbate this problem. The presence of aggregated protein, which may be nonspecifically toxic and immunogenic, and therefore must be removed for therapeutic applications, thus makes the scale-up process for the production of these conjugates more difficult and decreases the yield of the products.

The amount of calicheamicin loaded on the carrier protein (the drug loading), the amount of aggregate that is formed in the conjugation reaction, and the yield of final purified monomeric conjugate that can be obtained are all related. In some cases, it is often difficult to make conjugates in useful yields with useful loadings for therapeutic applications using the reaction conditions disclosed in U.S. Pat. No. 5,053,394 due to excessive aggregation. These reaction conditions utilized DMF as the co-solvent in the conjugation reaction. Methods that allow for higher drug loadings/yield without aggregation and the inherent loss of material are therefore needed. Improvements to reduce aggregation are described in U.S. Pat. Nos. 5,712,374 and 5,714,586, and U.S. Patent Application Nos. 2004/0082764 A1 and 2004/0192900 A1, which are incorporated herein in their entirety.

The tendency for calicheamicin conjugates to aggregate is especially problematic when the conjugation reactions are performed with the linkers described in U.S. Pat. Nos. 5,877,296 and 5,773,001, which are incorporated herein in their entirety. In this case, a large percentage of the conjugates produced are in an aggregated form, and it is quite difficult to purify conjugates made by these processes, e.g., using the process described in U.S. Pat. No. 5,877,296, for therapeutic use. For some carrier proteins, conjugates with even modest loadings are virtually impossible to make except on a small scale. This is especially true for antibodies wherein the antibody isotype and differential glycosylation patterns affect the conjugation process. Consequently, there is a need to devise new and improved methods for conjugating calicheamicins to particular antibodies, thereby minimizing the amount of aggregation and allowing for as high a drug loading as possible with a reasonable yield of product.

### BRIEF DESCRIPTION OF THE DRAWINGS

FIG. 1 shows the structure of an anti-Lewis Y antibody (hu3S193) conjugated to calicheamicin (hu3S193-AcBut-CM).

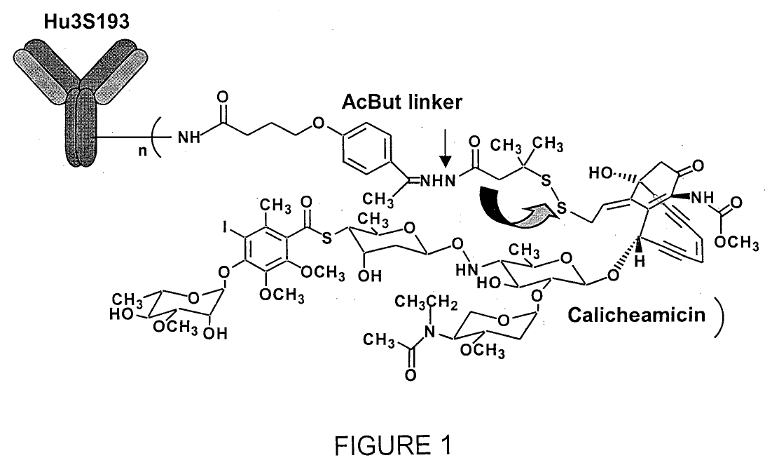

FIGS. 2A and 2B show the effect of an anti-Lewis Y antibody conjugated to calicheamicin (hu3S193-AcBut-CM) on Le^{y+ and -} cells as graphs of the frequency of occurrence versus the ED₅₀ (ng/ml); FIG. 2A shows the Le^{y+} cell line AGS and FIG. 2B shows the Le^{y-} cell line PC3MM2. FIG. 2C shows the effect of hu3S193-AcBut-CM on Le^{y+ and -} cells as a graph of the fold of CMA versus expression of Lewis Y on the surface of the cells (i.e., the Le^{y+} cell lines LOVO, N87, HCT8/S11-R1, AGS, LNCaP, NCl-H358 and the Le^{y-} cell lines PC3-MM2, A431, and PANC-1), with n representing the number of independent ED₅₀ determinations.

FIG. 3 shows the in vivo activity of an anti-Lewis Y antibody conjugated to calicheamicin (hu3S193-AcBut-CM) against N87 gastric carcinoma xenografts as graphs of tumor volume (cm³) versus period of tumor growth (days); FIG. 3A shows control conjugates CMA and RITUXAN®-AcBut-CM and FIG. 3B shows hu3S193 and hu3S193-AcBut-CM.

FIG. 4 shows the in vivo activity of an anti-Lewis Y antibody conjugated to calicheamicin (hu3S193-AcBut-CM) against LNCaP prostate carcinoma xenografts as a graph of tumor volume (cm³) versus period of tumor growth (days).

FIG. 5 shows the in vivo activity of an anti-Lewis Y antibody conjugated to calicheamicin (hu3S193-AcBut-CM) against LOVO colon carcinoma xenografts as graphs of tumor volume (cm³) versus period of tumor growth (days); FIG. 5A shows a control conjugate RITUXAN-AcBut-CM and FIG. 5B shows hu3S193 and hu3S193-AcBut-CM.

FIG. 6 shows the in vivo activity of an anti-Lewis Y antibody conjugated to calicheamicin (hu3S193-AcBut-CM) against LOVO colon carcinoma xenografts as graphs of tumor volume (cm³) versus period of tumor growth (days); FIG. 6A shows control conjugates CMA and RITUXAN-AcBut-CM and FIG. 6B shows hu3S193 and hu3S193-AcBut-CM administered at 4 μg Q4Dx3 or Q4Dx4.

FIG. 7 shows a comparison of the amino acid sequences of the mature secreted anti-Lewis Y antibodies hu3S193 (wt) and G193 (mt) IgG1 heavy chains in which the mutant sites are bolded and highlighted and the CDRs are bolded and shaded.

FIG. 8 shows a comparison of the amino acid sequences of the IgG1 heavy chains of hu3S193 (Wyeth wt) and hu3S193 (Ludwig Institute for Cancer Research hereinafter referred to as LICR wt) in which the CDRs are bolded and shaded and the allotypic differences are highlighted and bolded.

FIG. 9 shows the growth inhibition in vitro of A431 (FIG. 9A) and A431/Le^{y} (FIG. 9B) epidermoid carcinoma cells by an anti-Lewis Y antibody conjugated to calicheamicin (CMD-193) as a graph of the percent control (CMA) versus concentration of calicheamicin (cal. eq., ng/ml).

FIG. 10 shows the in vivo growth inhibition of anti-Lewis Y antibodies conjugated to calicheamicin (hu3S193-AcBut-CM and CMD-193) against N87 gastric carcinoma xenografts as graphs of tumor volume (cm³) versus period of tumor growth (days); FIG. 10A shows the control conjugate CMA, FIG. 10B shows CMD-193 and hu3S193-AcBut-CM, and FIG. 10C shows free antibody.

FIG. 11 shows the in vivo growth inhibition of anti-Lewis Y antibodies conjugated to calicheamicin (hu3S193-AcBut-CM and CMD-193) against L2987 lung carcinoma xenografts as graphs of tumor volume (cm³) versus period of tumor growth (days); FIG. 11A shows the control conjugate CMA and FIG. 11B shows CMD-193.

FIG. 12 shows the in vivo growth inhibition of anti-Lewis Y antibodies conjugated to calicheamicin (hu3S193-AcBut-CM and CMD-193) against L2987 lung carcinoma xenografts as graphs of the number of mice with a tumor volume less than the initial average volume of of each group (%) versus period of tumor growth (days); FIG. 12A shows the control conjugate CMA and FIG. 12B shows CMD-193.

FIG. 13 shows the in vivo growth inhibition of an anti-Lewis Y antibody conjugated to calicheamicin (CMD-193) against L2987 lung carcinoma xenografts as a graph of tumor volume (cm³) versus period of tumor growth (days).

FIG. 14 shows the in vivo growth inhibition of an anti-Lewis Y antibody conjugated to calicheamicin (CMD-193) against A431/Le^{y} epidermoid carcinoma xenografts as a graph of tumor volume (cm³) versus period of tumor growth (days).

FIG. 15 shows the in vivo growth inhibition of an anti-Lewis Y antibody conjugated to calicheamicin (CMD-193) against A431/Le^{y} epidermoid carcinoma xenografts as graphs of tumor volume (cm³) versus period of tumor growth (days); FIG. 15A shows the efficacy of the control conjugate CMA and FIG. 15B shows the efficacy of CMD.

FIG. 16 shows the in vivo growth inhibition of an anti-Lewis Y antibody conjugated to calicheamicin (CMD-193) against A431/Le^{y} epidermoid carcinoma xenografts as graphs of the number of mice with a tumor volume less than the initial average volume of of each group (%) versus period of tumor growth (days); FIG. 16A shows the efficacy of the control conjugate CMA and FIG. 16B shows the efficacy of CMD.

FIG. 17 shows the in vivo growth inhibition of an anti-Lewis Y antibody conjugated to calicheamicin (CMD-193) against LS174T colon carcinoma cellxenografts as graphs of tumor volume (cm³) versus period of tumor growth (days); FIG. 17A shows the efficacy of the control conjugate CMA and FIG. 17B shows the efficacy of CMD.

FIG. 18 shows the in vivo growth inhibition of anti-Lewis Y antibodies conjugated to calicheamicin (CMD-193 and hu3S193-AcBut-CM) against LOVO colon carcinoma xenografts as graphs of tumor volume (cm³) versus period of tumor growth (days); FIG. 18A shows the efficacy of the control conjugate CMA and G193, FIGS. 18B and 18C show the efficacy of CMD at Z4DX3 and Q4DX4, respectively, and FIGS. 18D and 18E show the efficacy of CMD at various time intervals.

FIG. 19 shows the survival of nude mice following injection with various doses of an anti-Lewis Y antibody conjugated to calicheamicin (CMD-193) as a graph of percent survival versus observation period (days).

FIG. 20 shows the binding specificity of an anti-Lewis Y antibody conjugated to calicheamicin (CMD-193) to the Lewis Y antigen as a graph of Lewis Y and structurally related antigens versus BIAcore resonance units.

FIG. 21 shows the in vivo activity of an anti-Lewis Y antibody conjugated to calicheamicin (hu3S193-AcBut-CM) against HCT8S11 colon carcinoma xenografts as graphs of tumor mass (g) versus period of tumor growth (days); FIG. 21A shows small tumors and FIG. 21B shows large tumors.

FIG. 22 shows the in vivo activity of an anti-Lewis Y antibody conjugated to calicheamicin (CMD-193) against MX1 breast carcinoma xenografts as a graph of relative tumor growth versus period of tumor growth (days).

FIG. 23 shows the in vivo activity based on different drug loadings of an anti-Lewis Y antibody conjugated to calicheamicin (CMD-193) against N87 gastric carcinoma xenografts as a graph of tumor mass (g) versus tumor growth period (days).

FIG. 24 shows the in vitro complement-dependent cytotoxicity (CDC) activity of an anti-Lewis Y antibody (G193) and its calicheamicin conjugate (CMD-193) against N87 gastric carcinoma cells as a graph of percent cytotoxicity versus antibody concentration (μg/ml).

FIG. 25 shows the in vitro antibody-dependent cellular cytotoxicity (ADCC) activity of an anti-Lewis Y antibody (G193) and its calicheamicin conjugate (CMD-193) against A431/Le^{y} epidermoid carcinoma cells; FIG. 25A shows activity against Lewis Y⁺⁺⁺ A431 carcinoma cells and FIG. 25B shows activity against Lewis Y negative A431 carcinoma cells.

### SUMMARY OF THE INVENTION

The present invention provides processes for preparing a calicheamicin conjugate comprising reacting at a pH of about 7 to about 9 (preferably about 8.2) (i) an activated calicheamicin-hydrolyzable linker derivative and (ii) an IgG1 antibody in the presence of a member of the deoxycholate family or a salt thereof, as well as conjugates prepared by this process. Also provided by the present invention are compositions comprising a conjugate of a calicheamicin-hydrolyzable linker derivative covalently attached to an anti-Lewis Y antibody.

In one embodiment, the deoxycholate family member has one of the following structures: 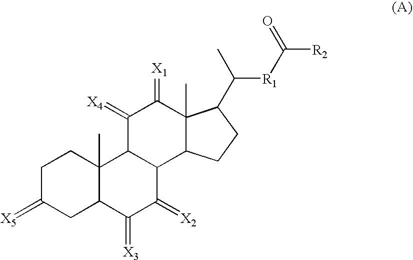
wherein
two of X₁ through X₅ are H or OH and the other three are independently either O or H;
R₁ is (CH₂)ₙ where n is 0-4 and
R₂ is OH, NH(CH₂)ₘCOOH, NH(CH₂)ₘSO₃H, or NH(CH₂)ₘPO₃H₂ where m is 1-4.
OR 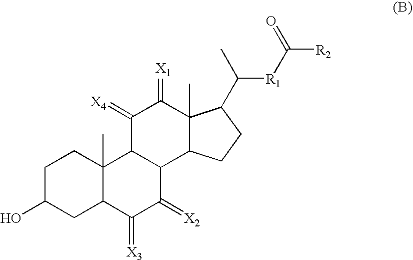
   wherein
one of X₁ through X₄ is H or OH and the other three are independently either O or H;
R₁ is (CH₂)ₙ where n is 0-2 and
R₂ is OH, NH(CH₂)ₘCOOH, or NH(CH₂)ₘSO₃H, where and m is 2.
OR 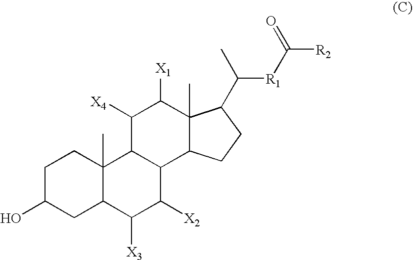
   wherein
one of X₁ through X₄ is OH and the other three are H;
R₁ is (CH₂)ₙ where n is 0-2 and
R₂ is OH, NH(CH₂)₂SO₃H.
   The deoxycholate family member can also be chenodeoxycholic acid, hyodeoxycholate, urosodeoxycholic acid, glycodeoxycholic acid, taurodeoxycholic acid, tauroursodeoxycholic, or taurochenodeoxycholic. Preferably, the deoxycholate family member is deoxycholic acid at a concentration of about 10 mM.

In another embodiment, the calicheamicin derivative is about 3 to about 9% by weight of the IgG1 antibody, preferably about 7% by weight of the IgG1 antibody.

The IgG1 antibody, in one embodiment, is an anti-Lewis Y antibody, which, preferably, is anti-Lewis Y antibody is G193 or Hu3S193.

In another embodiment, the calicheamicin derivative is an N-acyl derivative of calicheamicin or a disulfide analog of calicheamicin. Preferably, the calicheamicin derivative is N-acetyl gamma calicheamicin dimethyl hydrazide (N-acetyl calicheamicin DMH).

In yet another embodiment, the hydrolyzable linker is 4-(4-acetylephenoxy) butanoic acid (AcBut).

The process can optionally further comprise purifying the calicheamicin conjugate. Such purification can comprise chromatographic separation and ultrafiltration/diafiltration. Preferably, the chromatographic separation is size exclusion chromatography (SEC) or hydrophobic interaction chromatography (HIC). Following the purification step, preferably the average loading of the conjugate is from about 5 to about 7 moles of calicheamicin per mole of IgG1 antibody and the low conjugated fraction (LCF) of the conjugate is less than about 10%.

Calicheamicin conjugates of the present invention preferably have a K_{D} of about 1×10⁻⁷ M to about 4×10⁻⁷ M and, more preferably, a K_{D} of about 3.4×10⁻⁷ M. Such conjugates bind the Lewis Y antigen and do not bind Lewis X and H-2 blood group antigens, have cytotoxic activity, and have anti-tumor activity. Preferably, the conjugate is present in the composition in a therapeutically effective amount.

Thus, the present invention provides a composition comprising a conjugate of N-acetyl gamma calicheamicin dimethyl hydrazide-4-(4-acetylephenoxy) butanoic acid (N-acetyl calicheamicin DMH-AcBut) covalently linked to G193, wherein the average loading is from about 5 to about 7 moles of N-acetyl calicheamicin DMH per mole of G193 and the low conjugated fraction (LCF) of the conjugate is less than about 10%.

The present invention also provides a process for preserving biological activity of these compositions comprising: contacting the composition with a cryoprotectant, a surfactant, a buffering agent, and an electrolyte in a solution; and lyophilizing the solution.

In one embodiment, the conjugate is at a concentration of about 0.5 mg/mL to about 2 mg/mL. Preferably, the conjugate is at a concentration of 1 mg/mL.

In another embodiment, the cryoprotectant is at a concentration of about 1.5% to about 6% by weight. The cryoprotectant can be a sugar alcohol or a carbohydrate; preferably, the cryoprotectant is trehalose, mannitol, or sorbitol, and, more preferably, the the cryoprotectant is sucrose at a concentration of about 5%.

The surfactant in one embodiment is at a concentration of about 0.005% to about 0.05%. Preferably, the surfactant is Polysorbate 80 at a concentration of 0.01% by weight or Tween 80 at a concentration of about 0.01%.

In another embodiment, the buffering agent is at a concentration of about 5 mM to about 50 mM. Preferably, the buffering agent is Tris at a concentration of about 20 mM.

The electrolyte in another embodiment is at a concentration of about 5 mM to about 100 mM. Preferably, the electrolyte is a sodium or potassium salt and, more preferably, the electrolyte is NaCl at a concentration of about 10 mM.

Prior to lyophilization, in one embodiment, the pH is about 7.8 to about 8.2 and, preferably, the pH is about 8.0.

In one embodiment, lyophilization comprises: freezing the solution at a temperature of about −35° to about −50° C.; initially drying the frozen solution at a primary drying pressure of about 20 to about 80 microns at a shelf-temperature of about −10° to about −40° C. for 24 to 78 hours; and secondarily drying the freeze-dried product at a secondary drying pressure of about 20 to about 80 microns at a shelf temperature of about +100 to about +30° C. for 15 to 30 hours. Preferably, freezing is carried out at about −45° C.; the initial freeze drying is at a primary drying pressure of about 60 microns and a shelf temperature of about −30° C. for 60 hours; and the secondary drying step is at a drying pressure about 60 microns and a shelf temperature of about +25° C. for about 24 hours.

The process can optionally further comprises adding a bulking agent prior to lyophilization. Preferably, the bulking agent is at a concentration of about 0.5 to about 1.5% by weight and, more preferably, the bulking agent is Dextran 40 at a concentration of about 0.9% by weight or hydroxyethyl starch 40 at a concentration of about 0.9% by weight.

The present invention further provides a formulation comprising a calicheamicin-anti-Lewis Y antibody conjugate composition described above, a cryoprotectant, a surfactant, a buffering agent, and an electrolyte.

A method of treating cancer or another proliferative disorder is also provided by the present invention comprising administering a therapeutically effective amount of the compositions described herein, which can also be used in the manufacture of a medicament for treating cancer.

These compositions can be administered as a second-line monotherapy or as a first-line combination therapy.

Preferably, the cancer is positive for Lewis Y antigen and, more preferably, the cancer is a carcinoma. Also, preferably, the cancer is Non-Small Cell Lung Cancer (NSCLC), breast cancer, prostate cancer or colorectal cancer.

The methods of the present invention can be practiced in combination with a bioactive agent such as, for example, an anti-cancer agent.

Also provided by the present invention are kits comprising (i) a container which holds any of the formulations of the present invention; and (ii) instructions for reconstituting the formulation with a diluent to a conjugate concentration in the reconstituted formulation within the range from about 0.5 mg/mL to about 5 mg/mL.

### DETAILED DESCRIPTION OF THE INVENTION

The present invention provides processes for preparing calicheamicin conjugates. The processes involve reacting, at a pH of about 7 to about 9, (i) an activated calicheamicin-hydrolyzable linker derivative and (ii) an IgG1 antibody, e.g., an anti-Lewis Y antibody in the presence of a member of the deoxycholate family or a salt thereof, as well as conjugates produced thereby. Also provided by the present invention are compositions having conjugates of a calicheamicin-hydrolyzable linker covalently attached to an anti-Lewis Y antibody. Processes are also provided for preserving biological activity of these compositions involving contacting the composition with a cryoprotectant, a surfactant, a buffering agent, and an electrolyte in a solution; and lyophilizing the solution. Formulations of the calicheamicin-anti-Lewis Y antibody conjugates, a cryoprotectant, a surfactant, a buffering agent, and an electrolyte are further provided, as well as articles of manufacture. Finally, the present invention provides methods of treating cancer or other proliferative disorders by administering a therapeutically effective amount of such compositions/formulations, inlcluding uses of these compositions/formulations in the manufacture of medicaments for treatment of cancer or other proliferative diseases. Described below are various embodiments of the present invention.

Established conjugation conditions have been applied to the formation of MYLOTARG (referred to also as CMA-676 or CMA) and CMC-544, a humanized anti-CD22 antibody G5/44 calicheamicin conjugate. Both of these are IgG4 antibodies. Since the introduction of MYLOTARG, it has been learned, through the use of ion-exchange chromatography, that the calicheamicin is not distributed on these types of antibodies in a uniform or homogenous manner. Although the average loading of these conjugates is from 0.1 to 10 or 15 moles of calicheamicin per mole of antibody, most of the calicheamicin is on approximately half of the antibody, while the other half exists in a low conjugate fraction (LCF) that contains only small amounts of calicheamicin.

Improved methods for conjugating cytotoxic drugs such as calicheamicins to carriers, thereby minimizing the amount of aggregation and allowing for a higher uniform drug loading with a significantly improved yield of the conjugate product was accomplished during the development of CMC-544. A specific example is that of the G5/44-humanized anti-CD22 antibody-NAc-gamma calicheamicin DMH AcBut conjugate (i.e, CMC-544). The reduction of the amount of the LCF to <10% of the total antibody was desired for development of CMC-544, and various options for reduction of the levels of the LCF were considered. Other attributes of the immunoconjugate, such as antigen binding and cytotoxicity, must not be affected by the ultimate solution. The options considered included genetic or physical modification of the antibody molecule, chromatographic separation techniques, or modification of the reaction conditions.

Reaction of the G5/44 antibody with NAc-gamma calicheamicin-DMH-AcBut-OSu using the old reaction conditions (CMA-676 Process Conditions) resulted in a product with similar physical properties (drug loading, LCF) and aggregation as CMA-676. However, the high level (50-60%) of LCF present after conjugation was deemed undesirable. Optimal reaction conditions were determined through statistical experimental design methodology in which key reaction variables, such as temperature, pH, calicheamicin derivative input, and additive concentration, were evaluated. In order to reduce the LCF to <10%, the calicheamicin derivative input was increased from 3% to 8.5% (w/w) relative to the amount of antibody in the reaction. The additive was changed from octanoic acid or its salt at a concentration of 200 mM (CMA process) to decanoic acid or its salt at a concentration of 37.5 mM. The reaction conditions incorporating these changes reduced the LCF to below 10 percent while increasing calicheamicin loading, and is hereinafter referred to as CMC-544 Process Conditions.

The increase in calicheamicin input increased the drug loading from 2.5-3.0 weight percent to 5.0-9.0 (most typically 5.5-8.5) weight percent, and resulted in no increase in protein aggregation in the reaction. Due to reduction of aggregate and LCF, the CMC-544 Process Conditions resulted in a more homogeneous product.

Due to variations in amino acid sequence and isotype not all antibodies show the same physical characteristics and reaction conditions must be tailored to each specific antibody. When the CMA-676 conjugation reaction conditions were used with an IgG1 antibody, for example, an anti-Lewis Y antibody, the resulting conjugate had similar physical properties (drug loading, LCF, and aggregation) as CMA-676, and the high level (50-60%) of LCF present after conjugation was deemed undesirable. Using the modified conditions developed for CMC-544 with an IgG1 antibody resulted in a product with lower LCF, but the amount of aggregate produced in the reactions was considered too high. It was determined that specific bile acids, the deoxycholate family, or their salts worked as the best additives to reduce both LCF and aggregate in this instance. A comparison of one IgG1 antibody conjugate prepared with additives from both the CMA-676, CMC-544 and new optimized process is shown in Table 1 (Comparison of Octanoate, Decanoate and Deoxycholate).

TABLE 1

| Conditions/Results | Octanoate | Decanoate | Deoxycholate |
|---|---|---|---|
| Calicheamicin Derivative | 7.0% (w/w) | 7.0% (w/w) | 7.0% (w/w) |
| Input | | | |
| Additive Concentration | 200 mM | 37.5 mM | 10 mM |
| Temperature | 32 (±2)° C. | 32 (±2)° C. | 32 (±2)° C. |
| pH | 8.2 (±0.2) | 8.2 (±0.2) | 8.2 (±0.2) |
| Loading (μg | 65-75 | 65-75 | 65-75 |
| calicheamicin/mg antibody) | | | |
| Low Conjugate Fraction | 13.5% | 5.3% | 3.8% |
| Aggregation (End of | 25.4% | 14.6% | 3.2% |
| Reaction) | | | |

The present invention thus provides a process for preparing a calicheamicin conjugate. In this process, an activated calicheamicin-hydrolyzable linker derivative and an IgG1 antibody are reacted in the presence of a member of the deoxycholate family or a salt thereof. This process minimizes the amount of aggregation and significantly increases drug loading for IgG1 antibody conjugates.

Any suitable member of the deoxycholate family of bile acids or a salt thereof can be used in the present inventive process. In one embodiment, the deoxycholate family member has the following structure: 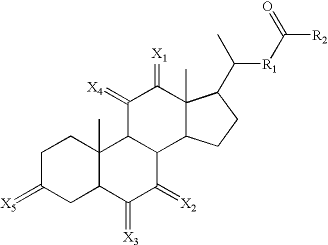
wherein
two of X, through X₅ are H or OH and the other three are independently either O or H;
R₁ is (CH₂)ₙ where n is 0-4 and
R₂ is OH, NH(CH₂)ₘCOOH, NH(CH₂)ₘSO₃H, or NH(CH₂)ₘPO₃H₂ where m is 1-4.
   Alternatively, the deoxycholate family member can have the following structure: 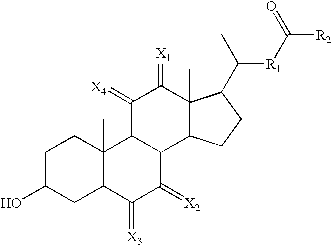
   wherein
one of X₁ through X₄ is H or OH and the other three are independently either O or H;
R₁ is (CH₂)ₙ where n is 0-2 and
R₂ is OH, NH(CH₂)ₘCOOH, or NH(CH₂)ₘSO₃H, where m is 2.
   Also alternatively, the deoxycholate family member can have the following structure: 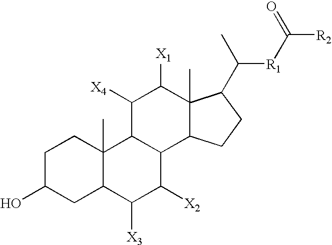
   wherein
one of X₁ through X₄ is OH and the other three are H;
R₁ is (CH₂)ₙ where n is 0-2 and
R₂ is OH, NH(CH₂)₂SO₃H.

Preferably, the deoxycholate family member is deoxycholic acid chenodeoxycholic acid, hyodeoxycholate, urosodeoxycholic acid, glycodeoxycholic acid, taurodeoxycholic acid, tauroursodeoxycholic acid, or taurochenodeoxycholic acid. More prefereably, the deoxycholate family member is deoxycholic acid, which is preferably present at a concentration of about 10 mM.

As discussed previously, calicheamicin refers to a family of antibacterial and antitumor agents, as described in U.S. Pat. No. 4,970,198 (see also U.S. Pat. No. 5,108,912). In one preferred embodiment of the present process, the calicheamicin is an N-acyl derivative of calicheamicin or a disulfide analog of calicheamicin. The dihydro derivatives of these compounds are described in U.S. Pat. No. 5,037,651 and the N-acylated derivatives are described in U.S. Pat. No. 5,079,233. Related compounds, which are also useful in this invention, include the esperamicins, described in U.S. Pat. Nos. 4,675,187; 4,539,203; 4,554,162; and 4,837,206. All of these compounds contain a methyltrisulfide that can be reacted with appropriate thiols to form disulfides, at the same time introducing a functional group such as a hydrazide or similar nucleophile. All information in the above-mentioned patent citations is incorporated herein by reference. Two compounds that are useful in the present invention are disclosed in U.S. Pat. No. 5,053,394, and are shown in Table 1 of U.S. Pat. No. 5,877,296, gamma dimethyl hydrazide and N-acetyl gamma dimethyl hydrazide.

Preferably, in the context of the present invention, the calicheamicin is N-acetyl gamma calicheamicin dimethyl hydrazide (N-acetyl calicheamicin DMH). N-acetyl calicheamicin DMH is at least 10- to 100-fold more potent than the majority of cytotoxic chemotherapeutic agents in current use. Its high potency makes it an ideal candidate for antibody-targeted therapy, thereby maximizing antitumor activity while reducing nonspecific exposure of normal organs and tissues.

Thus, in one embodiment, the conjugates of the present invention have the formula:
Pr(—X—W)ₘ
wherein:
Pr is an IgG1 antibody;
X is a linker that comprises a product of any reactive group that can react with the IgG1 antibody;
W is a cytotoxic drug from the calicheamicin family;
m is the average loading for a purified conjugation product such that the calicheamicin constitutes 3-9% of the conjugate by weight; and
(—X—W)ₘ is a cytotoxic drug derivative
Preferably, X has the formula
   (CO-Alk¹-Sp¹-Ar-Sp²-Alk²-C(Z¹)=Q-Sp)
   wherein
Alk¹ and Alk² are independently a bond or branched or unbranched (C₁-C₁₀) alkylene chain;
Sp¹ is a bond, —S—, —O—, —CONH—, —NHCO—, —NR—, —N(CH₂CH₂)₂N—, or —X—Ar—Y—(CH₂)ₙ-Z wherein X, Y, and Z are independently a bond, —NR—, —S—, or —O—, with the proviso that when n=0, then at least one of Y and Z must be a bond and Ar is 1,2-, 1,3-, or 1,4-phenylene optionally substituted with one, two, or three groups of (C₁-C₅) alkyl, (C₁-C₄) alkoxy, (C₁-C₄) thioalkoxy, halogen, nitro, —COOR, —CONHR, —(CH₂)ₙCOOR, —S(CH₂)ₙCOOR, —O(CH₂)ₙCONHR, or —S(CH₂)ₙCONHR, with the proviso that when Alk¹ is a bond, Sp¹ is a bond;
n is an integer from 0 to 5;
R is a branched or unbranched (C₁-C₅) chain optionally substituted by one or two groups of —OH, (C₁-C₄) alkoxy, (C₁-C₄) thioalkoxy, halogen, nitro, (C₁-C₃) dialkylamino, or (C₁-C₃) trialkylammonium -A⁻ where A⁻ is a pharmaceutically acceptable anion completing a salt;
Ar is 1,2-, 1,3-, or 1,4-phenylene optionally substituted with one, two, or three groups of (C₁-C₆) alkyl, (C₁-C₅) alkoxy, (C₁-C₄) thioalkoxy, halogen, nitro, —COOR, —CONHR, —O(CH₂)ₙCOOR, —S(CH₂)ₙCOOR, —O(CH₂)ₙCONHR, or —S(CH₂)ₙCONHR wherein n and R are as hereinbefore defined or a 1,2-, 1,3-, 1,4-, 1,5-, 1,6-, 1,7-, 1,8-, 2,3-, 2,6-, or 2,7-naphthylidene or 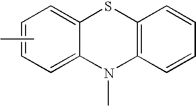
with each naphthylidene or phenothiazine optionally substituted with one, two, three, or four groups of (C₁-C₆) alkyl, (C₁-C₅) alkoxy, (C₁-C₄) thioalkoxy, halogen, nitro, —COOR, —CONHR, —O(CH₂)ₙCOOR, —S(CH₂)ₙCOOR, or —S(CH₂)ₙCONHR wherein n and R are as defined above, with the proviso that when Ar is phenothiazine, Sp¹ is a bond only connected to nitrogen;
Sp² is a bond, —S—, or —O—, with the proviso that when Alk² is a bond, Sp² is a bond;
Z¹ is H, (C₁-C₅) alkyl, or phenyl optionally substituted with one, two, or three groups of (C₁-C₅) alkyl, (C₁-C₅) alkoxy, (C₁-C₄) thioalkoxy, halogen, nitro, —COOR, —ONHR, —O(CH₂)ₙCOOR, —S(CH₂)ₙCOOR, —O(CH₂)ₙCONHR, or —S(CH₂)ₙCONHR wherein n and R are as defined above;
Sp is a straight or branched-chain divalent or trivalent (C₁-C₁₈) radical, divalent or trivalent aryl or heteroaryl radical, divalent or trivalent (C₃-C₁₈) cycloalkyl or heterocycloalkyl radical, divalent or trivalent aryl- or heteroaryl-aryl (C₁-C₁₈) radical, divalent or trivalent cycloalkyl- or heterocycloalkyl-alkyl (C₁-C₈) radical or divalent or trivalent (C₂-C₁₈) unsaturated alkyl radical, wherein heteroaryl is preferably furyl, thienyl, N-methylpyrrolyl, pyridinyl, N-methylimidazolyl, oxazolyl, pyrimidinyl, quinolyl, isoquinolyl, N-methylcarbazoyl, aminocourmarinyl, or phenazinyl and wherein if Sp is a trivalent radical, Sp can be additionally substituted by lower (C₁-C₅) dialkylamino, lower (C₁-C₅) alkoxy, hydroxy, or lower (C₁-C₅) alkylthio groups; and
Q is ═NHNCO—, ═NHNCS—, ═NHNCONH—, ═NHNCSNH—, or ═NHO—.

Preferably, Alk¹ is a branched or unbranched (C₁-C₁₀) alkylene chain; Sp is a bond, —S—, —O—, —CONH—, —NHCO—, or —NR wherein R is as hereinbefore defined, with the proviso that when Alk¹ is a bond, Sp¹ is a bond;
Ar is 1,2-, 1,3-, or 1,4-phenylene optionally substituted with one, two, or three groups of (C₁-C₆) alkyl, (C₁-C₅) alkoxy, (C₁-C₄) thioalkoxy, halogen, nitro, —COOR, —CONHR, —O(CH₂)ₙCOOR, —S(CH₂)ₙCOOR, —O(CH₂)ₙCONHR, or —S(CH₂)ₙCONHR wherein n and R are as hereinbefore defined, or Ar is a 1,2-, 1,3-, 1,4-, 1,5-, 1,6-, 1,7-, 1,8-, 2,3-, 2,6-, or 2,7-naphthylidene each optionally substituted with one, two, three, or four groups of (C₁-C₆) alkyl, (C₁-C₅) alkoxy, (C₁-C₄) thioalkoxy, halogen, nitro, —COOR, —CONHR, —O(CH₂)ₙCOOR, —S(CH₂)ₙCOOR, —O(CH₂)ₙCONHR, or —S(CH₂)ₙCONHR.

Z¹ is (C₁-C₅) alkyl, or phenyl optionally substituted with one, two, or three groups of (C₁-C₅) alkyl, (C₁-C₄) alkoxy, (C₁-C₄) thioalkoxy, halogen, nitro, —COOR, —CONHR, —O(CH₂)ₙCOOR, —S(CH₂)ₙCOOR, —O(CH₂)ₙCONHR, or —S(CH₂)ₙCONHR.
Alk² and Sp² are together a bond.
Sp and Q are as immediately defined above.

In the present process, the calicheamicin is preferably added to the reaction at about 3 to about 9% by weight of the IgG1 antibody and more preferably about 7% by weight of the IgG1 antibody.

The conjugates of the present invention utilize the cytotoxic drug calicheamicin derivatized with a linker that includes any reactive group which reacts with an IgG1 antibody, which is used as a proteinaceous carrier targeting agent to form a cytotoxic drug derivative-antibody conjugate. U.S. Pat. Nos. 5,773,001; 5,739,116 and 5,877,296, incorporated herein in its entirety, discloses linkers that can be used with nucleophilic derivatives, particularly hydrazides and related nucleophiles, prepared from the calicheamicins. These linkers are especially useful in those cases where better activity is obtained when the linkage formed between the drug and the linker is hydrolyzable. These linkers contain two functional groups. One group typically is a carboxylic acid that is utilized to react with the carrier. The acid functional group, when properly activated, can form an amide linkage with a free amine group of the carrier, such as, for example, the amine in the side chain of a lysine of an antibody or other proteinaceous carrier. The other functional group commonly is a carbonyl group, i.e., an aldehyde or a ketone, which will react with the appropriately modified therapeutic agent. The carbonyl groups can react with a hydrazide group on the drug to form a hydrazone linkage. This linkage is hydrolyzable, allowing for release of the therapeutic agent from the conjugate after binding to the target cells. Preferably, the hydrolyzable linker is 4-(4-acetylphenoxy) butanoic acid (AcBut).

N-hydroxysuccinimide (OSu) esters or other comparably activated esters can be used to generate the activated calicheamicin-hydrolyzable linker derivative. Examples of other suitable activating esters include NHS(N-hydroxysuccinimide), sulfo-NHS (sulfonated NHS), PFP (pentafluorophenyl), TFP (tetrafluorophenyl), and DNP (dinitrophenyl).

Examples of antibodies that may be used in the present invention include monoclonal antibodies (mAbs), for example, chimeric antibodies, humanized antibodies, primatized antibodies, resurfaced antibodies, human antibodies and biologically active fragments thereof. The term antibody, as used herein, unless indicated otherwise, is used broadly to refer to both antibody molecules and a variety of antibody derived molecules. Such antibody-derived molecules comprise at least one variable region (either a heavy chain or light chain variable region) and include molecules such as Fab fragments, F(ab′)₂ fragments, Fd fragments, Fabc fragments, Sc antibodies (single chain antibodies), diabodies, individual antibody light single chains, individual antibody heavy chains, chimeric fusions between antibody chains and other molecules, and the like.

Preferably the IgG1 antibodies of the present invention are directed against cell surface antigens expressed on target cells and/or tissues in proliferative disorders such as cancer. In one embodiment, the IgG1 antibody is an anti-Lewis Y antibody. Lewis Y is a carbohydrate antigen with the structure Fuca1→2Galβ1→4[Fuca1→3]GlcNacβ1→3R (Abe et al. (1983) J. Biol. Chem., 258 11793-11797). Lewis Y antigen is expressed on the surface of 60% to 90% of human epithelial tumors (including those of the breast, colon, lung, and prostate), at least 40% of which overexpress this antigen, and has limited expression in normal tissues.

In order to target Le^{y} and effectively target a tumor, an antibody with exclusive specificity to the antigen is ideally required. Thus, preferably, the anti-Lewis Y antibodies of the present invention do not cross-react with the type 1 structures (i.e., the lacto-series of blood groups (Le^{a} and Le^{b})) and, preferably, do not bind other type 2 epitopes (i.e., neolacto-structure) like Le^{x} and H-type 2 structures.

During the past decades, several antibodies that recognize Le^{y} have been generated. Most of these, however, show cross-reactivity with Le^{x} and type 2H-antigen structures (Furokawa, K., et al. 723-732). An example of a preferred anti-Lewis Y antibody is designated hu3S193 (see U.S. Pat. Nos. 6,310,185; 6,518,415; 5,874,060, incorporated herein in their entirety). Other examples of anti-Lewis Y antibodies (e.g., European Patent No. 0 285 059; U.S. Pat. Nos. 4,971,792 and 5,182,192) include the monoclonal antibody BR96 (e.g., U.S. Pat. Nos. 5,491,088; 5,792,456; 5,869,045), which is currently being evaluated as a doxorubicin conjugate in SGN-15 (e.g., U.S. Pat. No. 5,980,896), the monoclonal antibody of LMB-9 (B3(dsFv)PE38), which is a recombinant disulfide stabilized anti-Lewis Y IgGK immunotoxin containing a 38 kD toxic element derived from the Pseudomonas Aeruginosa exotoxin A (PE) (e.g., U.S. Pat. No. 5,980,895), and the IGN311 humanized antibody (e.g., European Patent No. 0 528 767 and U.S. Pat. No. 5,562,903).

The humanized antibody hu3S193 (Attia, M. A., et al. 1787-1800) was generated by CDR-grafting from 3S193, which is a murine monoclonal antibody raised against adenocarcinoma cell with exceptional specificity for Le^{y} (Kitamura, K., 12957-12961). Hu3S193 not only retains the specificity of 3S193 for Le^{y} but has also gained in the capability to mediate complement dependent cytotoxicity (hereinafter referred to as CDC) and antibody dependent cellular cytotoxicity (hereinafter referred to as ADCC) (Attia, M. A., et al. 1787-1800). This antibody targets Le^{y} expressing xenografts in nude mice as demonstrated by biodistribution studies with hu3S193 labeled with ¹²⁵I, ¹¹¹In, or ¹⁸F, as well as other radiolabels that require a chelating agent, such as ¹¹¹In, ^{99m}Tc, or ⁹⁰Y (Clark, et al. 4804-4811).

The subject invention provides for numerous humanized antibodies specific for the Lewis Y antigen based on the discovery that the CDR regions of the murine monoclonal antibody could be spliced into a human acceptor framework so as to produce a humanized recombinant antibody specific for the Lewis Y antigen. CDRs can be defined using any conventional nomenclature known in the art, such as the Kabat numbering system, the Chothia number system, or the AbM definition, which is a compromise between Kabat and Chothia used by Oxford Molecular's AbM antibody modeling software. Particularly preferred embodiments of the invention are the exemplified humanized antibody molecules that have superior antigen binding properties. The protocol for producing humanized recombinant antibodies specific for the Lewis Y antigen is set forth in U.S. Pat. No. 6,518,415, incorporated herein in its entirety. As discussed previously, in a preferred embodiment of the subject invention, the CDRs of the humanized Lewis Y specific antibody are derived from the murine antibody 3S193.

When the CDRs are grafted, any appropriate acceptor variable region framework sequence may be used having regard to the class/type of the donor antibody from which the CDRs are derived, including mouse, primate and human framework regions. Examples of human frameworks, which can be used in the present invention are KOL, NEWM, REI, EU, TUR, TEI, LAY and POM (Kabat et al. Seq. of Proteins of Immunol. Interest, 1:310-334 (1994)). For example, KOL and NEWM can be used for the heavy chain, REI can be used for the light chain and EU, LAY and POM can be used for both the heavy chain and the light chain.

In practice, for the generation of efficacious humanized antibodies retaining the specificity of the original murine antibody, it is not usually sufficient simply to substitute CDRs. There is a requirement for the inclusion of a small number of critical murine antibody residues in the human variable region frameworks. The identity of these residues depends on the structure of both the original murine antibody and the acceptor human antibody. Thus, the humanized antibodies described herein contain some alterations of the acceptor antibody, i.e., human, heavy and/or light chain variable domain framework regions that are necessary for retaining binding specificity of the donor monoclonal antibody. In other words, the framework region of some embodiments, the humanized antibodies described herein, does not necessarily consist of the precise amino acid sequence of the framework region of a naturally occurring human antibody variable region, but contains various substitutions that improve the binding properties of a humanized antibody region that is specific for the same target as the murine antibody 3S193. A minimal number of substitutions are made to the framework region in order to avoid large-scale introductions of non-human framework residues and to ensure minimal immunogenicity of the humanized antibody. Preferred anti-Lewis Y antibodies in the context of the present invention are thus hu3S193 and G193.

In one embodiment, variants of the antibody molecules of the present invention are directed against Lewis Y and display improved affinity for Lewis Y. Such variants can be obtained by a number of affinity maturation protocols including mutating the CDRs (Yang et al., J. Mol. Biol., 254, 392-403, 1995), chain shuffling (Marks et al., Bio/Technology, 10, 779-783, 1992), use of mutator strains of E. coli (Low et al., J. Mol. Biol., 250, 359-368, 1996), DNA shuffling (Patten et al., Curr. Opin. Biotechnol., 8, 724-733, 1997), phage display (Thompson et al., J. Mol. Biol., 256, 77-88, 1996) and PCR (Crameri et al., Nature, 391, 288-291, 1998).

The humanized antibodies of the subject invention may be produced by a variety of methods useful for the production of polypeptides, e.g., in vitro synthesis, recombinant DNA production, and the like. Preferably, the humanized antibodies are produced by recombinant DNA technology. The humanized Lewis Y specific antibodies of the invention thus can be produced by recombinant protein expression methods using DNA technology. Techniques for manipulating DNA (e.g., polynucleotides) are well known to the person of ordinary skill in the art of molecular biology. Examples of such well-known techniques can be found in Molecular Cloning: A Laboratory Manual 2^{nd} Edition, Sambrook et al, Cold Spring Harbor, N.Y. (1989). Techniques for the recombinant expression of immunoglobulins, including humanized immunoglobulins, can also be found, among other places in Goeddel et al, Gene Expression Technology Methods in Enzymology, Vol. 185, Academic Press (1991), and Borreback, Antibody Engineering, W. H. Freeman (1992). Additional information concerning the generation, design and expression of recombinant antibodies can be found in Mayforth, Designing Antibodies, Academic Press, San Diego (1993). Examples of conventional molecular biology techniques include, but are not limited to, in vitro ligation, restriction endonuclease digestion, PCR, cellular transformation, hybridization, electrophoresis, DNA sequencing, and the like.

The general methods for construction of the vector of the invention, transfection of cells to produce the host cell of the invention, culture of cells to produce the antibody of the invention are all conventional molecular biology methods. Likewise, once produced, the recombinant antibodies of the invention can be purified by standard procedures of the art, including cross-flow filtration, ammonium sulphate precipitation, affinity column chromatography, gel electrophoresis, diafiltration and the like. The host cells used to express the recombinant antibody may be either a bacterial cell, such as E. coli, or preferably, a eukaryotic cell. Preferably, a mammalian cell such as a PER.C.6 cell or a Chinese hamster ovary cell (CHO) is used. The choice of expression vector is dependent upon the choice of host cell, and is selected so as to have the desired expression and regulatory characteristics in the selected host cell.

Use of particular cosolvents, additives, and specific reaction conditions together with the separation process results in the formation of a monomeric cytotoxic drug derivative antibody conjugate with a significant reduction in the LCF. The monomeric form of the conjugates as opposed to the aggregated form has significant therapeutic value, and minimizing the LCF and substantially reducing aggregation results in the utilization of the antibody starting material in a therapeutically meaningful manner by preventing the LCF from competing with the more highly conjugated fraction (HCF).

In the context of the present invention, a monomeric cytotoxic drug conjugate refers to a single antibody covalently attached to any number of calicheamicin molecules without significant aggregation of the antibodies. The number of calicheamicin moieties covalently attached to an antibody is also referred to as drug loading. For example, according to the present invention, the average loading can be anywhere from 0.1 to 10 or 15 calicheamicin moieties per antibody. A given population of conjugates (e.g., in a composition or formulation) can be either heterogeous or homogenous in terms of drug loading. In a heterogenous population, since average loading represents the average number of drug molecules (or moles) conjugated to an antibody, the actual number of drug moieties per antibody can vary substantially. The percentage of antibody in a given population having unconjugated or significantly under-conjugated antibody is referred to as the low conjugate fraction or LCF.

The use of deoxycholate with a non-nucleophilic, protein-compatible, buffered solution was found to generally produce monomeric cytotoxic drug derivative derivative/carrier conjugates with higher drug loading/yield and decreased aggregation having excellent activity. Preferred buffered solutions for conjugates made from N-hydroxysuccinimide (OSu) esters or other comparably activated esters are phosphate-buffered saline (PBS), N-(2-Hydroxyethyl)piperazine-N-(4-butanesulfonic acid) (HEPBS), or N-2-hydroxyethyl piperazine-N-2-ethanesulfonic acid (HEPES buffer). The buffered solution used in such conjugation reactions cannot contain free amines or nucleophiles. Those who are skilled in the art can readily determine acceptable buffers for other types of conjugates.

The amount of additive necessary to effectively form a monomeric conjugate also varies from antibody to antibody. This amount can also be determined by one of ordinary skill in the art without undue experimentation. In the present reactions, the concentration of antibody can range from 1 to 15 mg/ml and the concentration of the calicheamicin derivative, e.g., N-acetyl gamma-calicheamicin DMH AcBut OSu ester, ranges from about 3-9% by weight of the antibody.

The cosolvent can alternatively be ethanol, for which good results have been demonstrated at concentrations ranging from 6 to 11.4% (volume basis). The reactions can be performed in PBS, HEPES, N-(2-Hydroxyethyl)piperazine-N-(4-butanesulfonic acid) (HEPBS), or other compatible buffer at a pH of about 7 to about 9, preferably 8 to 9, at a temperature ranging from about 25° C. to about 40° C., preferably about 30° C. to about 35° C., and for times ranging from 15 minutes to 24 hours. More preferably, the reaction is carried out at a pH of about 8.2. Those who are skilled in the art can readily determine acceptable pH ranges for other types of conjugates. For various antibodies the use of slight variations in the combinations of the aforementioned additives have been found to improve drug loading and monomeric conjugate yield, and it is understood that any particular antibody may require some minor alterations in the exact conditions or choice of additives to achieve the optimum results.

Following conjugation, the monomeric conjugates may be purified from unconjugated reactants (such as proteinaceous carrier molecules/antibodies and free cytotoxic drug/calicheamicin) and/or aggregated form of the conjugates. Conventional methods for purification, for example, size exclusion chromatography (SEC), hydrophobic interaction chromatography (HIC), ion exchange chromatography (IEC), chromatofocusing (CF), can be used. Following, for example, chromatographic separation, the conjugate can be ultrafiltered and/or diafiltered.

The purified conjugates are monomeric and usually contain from 3 to 9% by weight cytotoxic drug/calicheamicin. In a preferred embodiment, the conjugates are purified using HIC. When a cytotoxic drug has a highly hydrophobic nature, such as a calicheamicin derivative, and is used in a conjugate, HIC is a preferred candidate to provide effective separation of conjugated and unconjugated antibody. HIC presents three key advantages over SEC: (1) it has the capability to efficiently reduce the LCF content as well as aggregate; (2) the column load capacity for HIC is much higher; and (3) HIC avoids excessive dilution of the product. A number of high-capacity HIC media, suitable for production scale use, such as Butyl, Phenyl and Octyl Sepharose 4 Fast Flow (Amersham Biosciences, Piscataway, N.J.) could effectively separate unconjugated and aggregates of the conjugate from monomeric conjugated components following conjugation process.

Preferably, the HIC is carried out using Butyl Sepharose FF resin with a loading and wash buffer of 0.60 M potassium phosphate and an elution buffer of 20 mM Tris/25 mM NaCl. Also preferably, ultrafiltration is carried out using a regenerated cellulose membrane and diafiltration is carried out using 10 diavolumes of 20 mM Tris/10 mM NaCl buffer at a pH of 8.0.

Thus, according to the present inventive process, following the purification step, the average loading of the conjugate is from about 5 to about 7 moles of calicheamicin per mole of IgG1 antibody. In addition, following the purification step, the low conjugated fraction (LCF) of the conjugate is less than about 10%.

The present invention also provides conjugates prepared by these processes. Such conjugates preferably maintain the binding kinetics and specificity of the naked antibody. As such, the conjugates of the present invention preferably have a K_{D} of about about 100 to 400 nM, preferably 3.4×10⁻⁷ M, as determined by BIAcore analysis, bind the Lewis Y antigen and do not bind the Lewis X and H-2 blood group antigens, have cytotoxic activity, and/or have anti-tumor activity. Any known method can be used to determine the binding kinetics and specificty of the conjugate, such as FACS or BIAcore analysis, for example.

A preferred calicheamicin conjugate prepared by the process of the present invention is N-acetyl gamma calicheamicin dimethyl hydrazide (N-acetyl calicheamicin DMH) covelently attached to the hydrolyzable linker 4-(4-acetylphenoxy) butanoic acid (AcBut), covelently attached to the anti-Lewis Y antibody G193 (referred to variously as CMD-193 or CMD) with the average loading of the calicheamicin conjugate from about 5 to about 7 moles of calicheamicin per mole of antibody and the low conjugated fraction (LCF) of the conjugate less than about 10%.

Also provided by the present invention are compositions comprising a conjugate of a calicheamicin-hydrolyzable linker covalently attached to an anti-Lewis Y antibody together with a pharmaceutically acceptable excipient, diluent or carrier. Thus, a preferred composition according to the present invention comprises a conjugate of N-acetyl gamma calicheamicin dimethyl hydrazide-4-(4-acetylphenoxy) butanoic acid (N-acetyl calicheamicin DMH-AcBut) covalently linked to G193, wherein the average loading is from about 5 to about 7 moles of N-acetyl calicheamicin DMH per mole of G193 and the low conjugated fraction (LCF) of the conjugate is less than about 10%.

The humanized Lewis Y specific antibodies can be used in conjunction with, or attached to other antibodies (or parts thereof) such as human or humanized monoclonal antibodies. These other antibodies may be reactive with other markers (epitopes) characteristic for the disease against which the antibodies of the invention are directed or may have different specificities chosen, for example, to recruit molecules or cells of the human immune system to the diseased cells. The antibodies of the invention (or parts thereof) may be administered with such antibodies (or parts thereof) as separately administered compositions or as a single composition with the two agents linked by conventional chemical or by molecular biological methods. Additionally, the diagnostic and therapeutic value of the antibodies of the invention may be augmented by labeling the humanized antibodies with labels that produce a detectable signal (either in vitro or in vivo) or with a label having a therapeutic property. Some labels, e.g., radionuclides may produce a detectable signal and have a therapeutic property. Examples of radionuclide labels include ¹²⁵I, ^{1311,} ¹⁴C. Examples of other detectable labels include a fluorescent chromophore, such as fluorescein, phycobiliprotein or tetraethyl rhodamine for fluorescence microscopy, an enzyme which produces a fluorescent or colored product for detection by fluorescence, absorbance visible color or agglutination, which produces an electron dense product for demonstration by electron microscopy; or an electron dense molecule such as ferritin, peroxidase or gold beads for direct or indirect electron microscopic visualization. Labels having therapeutic properties include drugs for the treatment of cancer, such as methotrexate and the like.

The monomeric cytotoxic drug derivative/carrier conjugate may be the sole active ingredient in the therapeutic or diagnostic composition/formulation or may be accompanied by other active ingredients (e.g., chemotherapy agents, hormone therapy agents, or biological therapy agents described below), including other antibody ingredients, for example, anti-CD19, anti-CD20, anti-CD33, anti-T cell, anti-IFNγ or anti-LPS antibodies, or non-antibody ingredients such as cytokines, growth factors, hormones, anti-hormones, cytotoxic drugs and xanthines.

These compositions/formulations can be administered to patients for treatment of cancer. According to the present invention, a therapeutically effective amount of a composition or formulation of a calicheamicin-anti-Lewis Y antibody conjugate, a cryoprotectant, a surfactant, a buffering agent, and an electrolyte is administered to a patient in need thereof. Alternatively, the compostition or formulation is used to manufacture a medicament for treatment of cancer. It should be appreciated that this method or medicament can be used to treat any patient with a proliferative disorder characterized by cells expressing Lewis Y antigen on their surface. Thus, in one embodiment, the cancer treated is positive for Lewis Y antigen. The cancer is preferably one that expresses a high number of the Lewis Y antigen (i.e., high Lewis Y-expressing tumors). The cancer treated can be a carcinoma and, preferably, is Non-Small Cell Lung Cancer (NSCLC) or breast cancer or, alternatively, prostate cancer or colorectal cancer.

Preferably, hu3S193-AcBut-CM or CMD-193 can be utilized in any therapy where it is desired to reduce the level of cells expressing Lewis Y that are present in the subject being treated with the composition or medicament disclosed herein. Specifically, the composition or medicament is used to treat humans or animals with proliferative disorders namely carcinomas which express Lewis Y antigen on the cell surface. These Lewis Y expressing cells may be circulating in the body or be present in an undesirably large number localized at a particular site in the body.

The present treatment methods can be used in combination with other cancer treatments, including surgery, radiation, chemotherapy, hormone therapy, biologic therapies, bone marrow transplantation (for leukemias and other cancers where very high doses of chemotherapy are needed). New treatments are also currently being developed and approved based on an increased understanding of the biology of cancer.

Two general classes of radiation therapy exist and can be used in the present methods. In one class, brachytherapy, direct implants of a radioisotope are made into the tumor to deliver a concentrated dose to that area. In the other class, teletherapy, a beam is used to deliver radiation to a large area of the body or to the whole body in total body irradiation (TBI).

Any suitable chemothepeutic agent can be used in the present methods. These chemotherapeutic agents generally fall into the following classes (with examples of each): antimetabolites (e.g., folic acid antagonists such as methotrexate, purine antagonists such as 6-mercaptopurine (6-MP), and pyrimidine antagonists such as 5-fluorouracil (5-FU)); alkylating agents (cyclophosphamide); DNA binding agents (cisplatin or oxaliplatin); anti-tumor antibiotics (doxorubicin or mitoxantrone); mitotic inhibitors (e.g., the taxanes or microtubule inhibitors such as vincristine) or topoisomerase inhibitors (camptothecan or taxol). More specific examples are described below.

Hormone therapies relevant to the present methods include, for example, corticosteroids for leukemias and myelomas, estrogens and anti-estrogens for breast cancers, and androgens and anti-androgens for prostate cancer.

Biologic therapy uses substances derived from the body. Examples of suitable therapies in the present methods include antibodies (e.g., anti-EGFR antibodies, such as cetuximab or trastuzumab, or anti-VEGF antibodies, such as bevacizumab), T-cell therapies, interferons, interleukins, and hematopoietic growth factors.

Bone marrow transplantation can be used for treatment of some cancers, notably leukemias. To treat leukemias, the patient's marrow cells are destroyed by chemotherapy or radiation treatment. Bone marrow from a donor that has matching or nearly matching HLA antigens on the cell surface is then introduced into the patient. Bone marrow transplantation is also used to replace marrow in patients who required very high doses of radiation or chemotherapy to kill the tumor cells. Transplants are classified based on donor source. In allogeneic transplants, the marrow donor is often not genetically related but has matches with at least five out of six cell surface antigens that are the major proteins recognized by the immune system (HLA antigens). In autologous transplantation, patients receive their own marrow back after chemotherapy or radiation treatment. This type of bone marrow transplant can be used for non-marrow related cancers for which conventional treatment doses have been incompletely effective.

Additionally, new emerging approaches that can be used in the present methods, some of which are approved or in clinical trials, are being developed based on an increased understanding of the molecular and cellular bases of cancer and the progression of the disease. Protein kinase inhibitors (both small molecules and antibodies) that inhibit the phosphorylation cascade can be used (e.g., erlotinib or imatinib mesylate). Any antimetastasis agent can be used that blocks the spread of cancer cells and the invasion of new tissues. Antiangiogensis agents can be used that block development of blood vessels that nourish a tumor (e.g, thalidomide). Other agents that can be used are antisense oligonucleotides, which block production of aberrant proteins that cause proliferation of tumor cells. Gene therapy can also be used to introduce genes into T cells that are injected into the patient and are designed to kill specific tumor cells. Also, p53 can be targeted by introducing normal p53 genes into mutant cancer cells, for example, to re-establish sensitivity to chemotherapeutic drugs.

In one embodiment, the compositions/formulations of the present invention are used in combination with bioactive agents. Bioactive agents commonly used include antibodies, growth factors, hormones, cytokines, anti-hormones, xanthines, interleukins, interferons, cytotoxic drugs and antiangiogenic proteins.

Bioactive cytotoxic drugs commonly used to treat proliferative disorders such as cancer, and which may be used together with the calicheamicin-anti-Lewis Y antibody conjugates include: anthracyclines such as doxorubicin, daunorubicin, idarubicin, aclarubicin, zorubicin, mitoxantrone, epirubicin, carubicin, nogalamycin, menogaril, pitarubicin, and valrubicin for up to three days; pyrimidine or purine nucleosides such as cytarabine, gemcitabine, trifluridine, ancitabine, enocitabine, azacitidine, doxifluridine, pentostatin, broxuridine, capecitabine, cladribine, decitabine, floxuridine, fludarabine, gougerotin, puromycin, tegafur, tiazofurin; alkylating agents such as cyclophosphamide, melphalan, thiotepa, ifosfamide, carmustine, cisplatin, CKD-602, ledoxantrone, rubitecan, topotecan hydrochloride, LE-SN38, afeletecan hydrochloride, XR-11576 and XR-11612; antimetabolites such as methotrexate, 5 flurouracil, tegafur/uracil (UFT), ralititrexed, capecitabine, leucovorin/UFT, S-1, pemetrexed disodium, tezacitabine, trimetrexate glucuronate, thymectacin, decitabine; antitumor antibodies such as edrecolomab, mitomycin, mitomycin C and oxaliplatin; vinca alkyloids such as vincristine, vinblastine, vinorelbine, anhydrovinblastine; angiogenesis inhibitors such as vatalanib succinate, oglufanide, RPI-4610; signal transduction inhibitors such as gefitinib, 317615.2 HCL, indisulam, lapatinib, sorafenib, WHI-P131; apoptosis inducers such as alvocidib hydrochloride, irofulven, sodium phenylbutyrate, bortezomib, exisulind, MS-2167; epipodophyllotoxins such as etoposide; and taxanes such as paclitaxel, doceltaxel, DHA-paclitaxel, ixabepilone, polyglutamate paclitaxel, or epothilones.

Other chemotherapeutic/antineoplastic agents that may be administered in combination with hu3S193-AcBut-CM or CMD-193 or AG G193-AcBut-CM include adriamycin, cisplatin, carboplatin, cyclophosphamide, dacarbazine, ifosfamide, vindesine, gemcitabine, edatrexate, irinotecan, mechlorethamine, altretamine, carboplatine, teniposide, topotecan, gemcitabine, thiotepa, fluxuridine (FUDR), MeCCNU, vinblastine, vincristine, mitoxantrone, bleomycin, mechlorethamine, prednisone, procarbazine methotrexate, flurouracils, etoposide, taxol and its various analogs, mitomycin, thalidomide and its various analogs, GBC-590, troxacitabine, ZYC-300, TAU, (R) flurbiprofen, histamine hydrochloride, tariquidar, davanat-1, ONT-093. Administration may be concurrently with one or more of these therapeutic agents or, alternatively, sequentially with one or more of these therapeutic agents.

Bioactive antibodies that can be administered with the antibody conjugates of this invention include, but are not limited to Herceptin, Zevalin, Bexxar, Campath, cetuximab, bevacizumab, ABX-EGF, MDX-210, pertuzumab, trastuzumab, I-131 ch-TNT-1/b, hLM609, 6H9, CEA-Cide Y90, IMC-1C11, ING-1, sibrotuzumab, TRAIL-R1 Mab, YMB-1003, 2C5, givarex and MH-1.

The calicheamicin-anti-Lewis Y antibody conjugates may also be administered alone, concurrently, or sequentially with a combination of other bioactive agents such as growth factors, cytokines, steroids, antibodies such as anti-Lewis Y antibody, rituximab and chemotherapeutic agents as a part of a treatment regimen. Calicheamicin-anti-Lewis Y antibody conjugates may also be administered alone, concurrently, or sequentially with any of the above identified therapy regimens as a part of induction therapy phase, a consolidation therapy phase and a maintenance therapy phase.

The conjugates of the present invention may also be administered together with other bioactive and chemotherapeutic agents as a part of combination chemotherapy regimen for the treatment of relapsed aggressive carcinoma. Such a treatment regimen includes: CAP (Cyclophosphamide, Doxorubicin, Cisplatin), PV (Cisplatin, Vinblastine or vindesine), CE (Carboplatin, Etoposide), EP (Etoposide, Cisplatin), MVP (Mitomycin, Vinblastine or Vindesine, Cisplatin), PFL (Cisplatin, 5-Flurouracil, Leucovorin), IM (Ifosfamide, Mitomycin), IE (Ifosfamide, Etoposide); IP (Ifosfamide, Cisplatin); MIP (Mitomycin, Ifosfamide, Cisplatin), ICE (Ifosfamide, Carboplatin, Etoposide); PIE (Cisplatin, Ifosfamide, Etoposide); Viorelbine and Cisplatin; Carboplatin and Paclitaxel; CAV (Cyclophosphamide, Doxorubicin, Vincristine), CAE (Cyclophosphamide, Doxorubicin, Etoposide); CAVE (Cyclophosphamide, Doxorubicin, Vincristine, Etoposide); EP (Etoposide, Cisplatin); CMCcV (Cyclophosphamide, Methotrexate, Lomustine, Vincristine); CMF (Cyclophosphamide, Methotrexate, 5-Flurouracil); CAF (Cyclophosphamide, Doxorubicin, 5-Flurouracil); CEF (Cyclophosphamide, Epirubicin, 5-Flurouracil); CMFVP (Cyclophosphamide, Methotrexate, 5-Flurouracil, Vincristine, Prednisone); AC (Doxorubicin, Cyclophosphamide); VAT (Vinblastine, Doxorubicin, Thiotepa); VATH (Vinblastine Doxorubicin, Thiotepa, Fluosymesterone); CDDP+VP-16 (Cisplatin, Etoposide, Mitomycin C+Vinblastine).

The present invention also provides a method of treating human or animal subjects suffering from, or at risk of, a proliferative disorder characterized by cells expressing Lewis Y, the method comprising administering to the subject an effective amount of calicheamicin-anti-Lewis Y antibody conjugates of the present invention. It should be appreciated that by treating is meant inhibiting, preventing, or slowing cancer growth, including delayed tumor growth and inhibition of metastasis.

The compositions/formulations of the present invention can be administered as a second-line monotherapy. By second-line is meant that the present compositions/formulations are used after treatment with a different anti-cancer treatment, examples of which are described above. Alternatively, the compositions or formulations can be administered as a first-line combination therapy with another anti-cancer treatment described above.

The humanized antibody compositions of the invention may be administered to a patient in a variety of ways. Direct delivery of the compositions will generally be accomplished by injection, subcutaneously, intraperitoneally, intravenously or intramuscularly, or delivered to the interstitial space of a tissue. Preferably, the pharmaceutical compositions may be administered parenterally, i.e., subcutaneously, intramuscularly or intravenously. The compositions can also be administered into a lesion. Dosage treatment may be a single dose schedule or a multiple dose schedule.

Thus, this invention provides compositions/formulations for parenteral administration that comprise a solution of the human monoclonal antibody or a cocktail thereof dissolved in an acceptable carrier, preferably an aqueous carrier. For example, formulations of a calicheamicin-anti-Lewis Y antibody conjugate, a cryoprotectant, a surfactant, a buffering agent, and an electrolyte.

A variety of aqueous carriers can be used, e.g., water, buffered water, 0.4% saline, 0.3% glycine and the like. These solutions are sterile and generally free of particulate matter. These compositions may be sterilized by conventional, well-known sterilization techniques. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate. The concentration of antibody in these formulations can vary widely, e.g., from less than about 0.5%, usually at or at least about 1% to as much as 15 or 20% by weight and will be selected primarily based on fluid volumes and viscosities, for example, in accordance with the particular mode of administration selected.

It will be appreciated that the active ingredient in the composition will be an anti-Lewis Y antibody-calicheamicin conjugate. As such, it will be susceptible to degradation in the gastrointestinal tract. Thus, if the composition is to be administered by a route using the gastrointestinal tract, the composition will need to contain agents which protect the proteinaceous carrier from degradation but which release the conjugate once it has been absorbed from the gastrointestinal tract.

Actual methods for preparing parenterally administrable compositions and adjustments necessary for administration to subjects will be known or apparent to those skilled in the art and are described in more detail in, for example, Remingtons Pharmaceutical Science, 15^{th} Ed., Mack Publishing Company, Easton, Pa. (1980), which is incorporated herein by reference. A thorough discussion of pharmaceutically acceptable carriers is available in Remingtons Pharmaceutical Sciences (Mack Publishing Company, N.J. 1991).

Compositions may be administered individually to a patient or may be administered in combination with other agents, drugs or hormones. Cytokines and growth factors that may be used to treat proliferative disorders such as cancer, and which may be used together with the cytotoxic drug derivative/carrier conjugates of the present invention include interferons, interleukins such as interleukin 2 (IL-2), TNF, CSF, GM-CSF and G-CSF. Hormones commonly used to treat proliferative disorders such as cancer and which may be used together with the cytotoxic drug derivative/carrier conjugate of the present invention include estrogens (diethylstilbestrol, estradiol), androgens (testosterone, Halotestin), progestins (Megace, Provera), and corticosteroids (prednisone, dexamethasone, hydrocortisone). Antihormones such as antiestrogens (tamoxifen), antiandrogens (flutamide) and antiadrenal agents are commonly used to treat proliferative disorders such as cancer, and may be used together with the cytotoxic drug derivative/carrier conjugate of the present invention.

In addition, chemotherapeutic/antineoplastic agents commonly used to treat proliferative disorders such as cancer, and which may be used together with the cytotoxic drug derivative/carrier conjugate of the present invention include, but are not limited to Adriamycin, cisplatin, carboplatin, vinblastine, vincristine, bleomycin, methotrexate, doxorubicin, flurouracils, etoposide, taxol and its various analogs, mitomycin, thalidomide and its various analogs.

The pharmaceutical compositions/formulations should preferably comprise a therapeutically effective amount of the conjugate of the invention. The term therapeutically effective amount as used herein refers to an amount of a therapeutic agent needed to treat, ameliorate or prevent a targeted disease or condition, or to exhibit a detectable therapeutic or preventative effect. For any conjugate, the therapeutically effective dose can be estimated initially either in cell culture assays or in animal models, usually in rodents, rabbits, dogs, pigs or primates. The animal model may also be used to determine the appropriate concentration range and route of administration. Such information can then be used to determine useful doses and routes for administration in humans.

The precise effective amount for a human subject will also depend upon the nature and severity of the disease state, the general health of the subject, the age, weight and gender of the subject, diet, time and frequency of administration, drug combination(s), reaction sensitivities and tolerance/response to therapy. If the conjugate is being used prophylactically to treat an existing condition, this will also affect the effective amount. This amount can be determined by routine experimentation and is within the judgment of the clinician. Generally, an effective dose will be from 0.01 mg/m² to 50 mg/m², preferably 0.1 mg/m² to 20 mg/m², more preferably about 10-15 mg/m², calculated on the basis of the proteinaceous carrier.

The frequency of dose will depend on the half-life of the conjugate and the duration of its effect. If the conjugate has a short half-life (e.g., 2 to 10 hours) it may be necessary to give one or more doses per day. Alternatively, if the conjugate molecule has a long half-life (e.g., 2 to 15 days) it may only be necessary to give a dosage once per day, once per week or even once every 1 or 2 months.

A composition can also contain a pharmaceutically acceptable carrier for administration of the antibody conjugate. A pharmaceutical carrier can be any compatible, non-toxic substance suitable for delivery of the monoclonal antibodies to the patient. Sterile water, alcohol, fats, waxes, and inert solids may be included in the carrier. The carrier should not itself induce the production of antibodies harmful to the individual receiving the composition and should not be toxic. Suitable carriers may be large, slowly metabolized macromolecules such as proteins, polypeptides, liposomes, polysaccharides, polylactic acids, polyglycolic acids, polymeric amino acids, amino acid copolymers and inactive virus particles. Pharmaceutically accepted adjuvants (buffering agents, dispersing agent) may also be incorporated into the pharmaceutical composition.

Pharmaceutically acceptable salts can be used, for example, mineral acid salts, such as hydrochlorides, hydrobromides, phosphates and sulfates, or salts of organic acids, such as acetates, propionates, malonates and benzoates.

Pharmaceutically acceptable carriers in therapeutic compositions/formulations may additionally contain liquids such as water, saline, glycerol, and ethanol. Auxiliary substances, such as wetting or emulsifying agents or pH buffering substances, may be present in such compositions. Such carriers enable the compositions to be formulated as tablets, pills, dragees, capsules, liquids, gels, syrups, slurries and suspensions, for ingestion by the patient.

Preferred forms for administration include forms suitable for parenteral administration, e.g., by injection or infusion, for example, by bolus injection or continuous infusion. Where the product is for injection or infusion, it may take the form of a suspension, solution or emulsion in an oily or aqueous vehicle and it may contain formulatory agents, such as suspending, preserving, stabilizing and/or dispersing agents.

Although the stability of the buffered conjugate solutions is adequate for a short time, long-term stability is poor. To enhance stability of the conjugate and to increase its shelf life, the antibody-drug conjugate may be lyophilized to a dry form, for reconstitution before use with an appropriate sterile liquid. The problems associated with lyophilization of a protein solution are well documented. Loss of secondary, tertiary and quaternary structure can occur during freezing and drying processes. Contacting them with a cryoprotectant, a surfactant, a buffering agent, and an electrolyte in a solution and then lyophilizing the solution can preserve biological activity of these compositions/formulations. A lyoprotectant also can be added to the solution.

A stable formulation is one in which the antibody therein essentially retains its physical and chemical stability and integrity upon storage. Various analytical techniques for measuring antibody stability are available in the art and are reviewed in Peptide and Protein Drug Delivery, 247-301, Vincent Lee Ed., Marcel Dekker, Inc., New York, N.Y., Pubs. (1991) and Jones, A. Adv. Drug Delivery Rev. 10: 29-90 (1993). Stability can be measured at a selected temperature for a selected time period. For rapid screening, the formulation may be kept at 40° C. for 2 weeks to 1 month, at which time stability is measured. Where the formulation is to be stored at 2-8° C., generally the formulation should be stable at 30° C. or 40° C. for at least 1 month and/or stable at 2-8° C. for at least 2 years. Where the formulation is to be stored at 30° C., generally the formulation should be stable for at least 2 years at 30° C. and/or stable at 40° C. for at least 6 months. The extent of aggregation following lyophilization and storage can be used as an indicator of antibody stability. For example, a stable formulation may be one wherein less than about 10% and preferably less than about 5% of the antibody is present as an aggregate in the formulation. In other embodiments, any increase in aggregate formation following lyophilization and storage of the lyophilized formulation can be determined. For example, a stable lyophilized formulation may be one wherein the increase in aggregate in the lyophilized formulation is less than about 5% and preferably less than about 3%, when the lyophilized formulation is stored at 2-8° C. for at least one year. Furthermore, stability of the antibody formulation may be measured using a biological activity assay.

Cryoprotectants may have to be included to act as an amorphous stabilizer of the conjugate and to maintain the structural integrity of the protein during the lyophilization process. In one embodiment, the cryoprotectant useful in the present invention is a sugar alcohol, such as alditol, mannitol, sorbitol, inositol, polyethylene glycol and combinations thereof. In another embodiment, the cryoprotectant is a sugar acid, including an aldonic acid, an uronic acid, an aldaric acid, and combinations thereof.

The cryoprotectant of this invention may also be a carbohydrate. Suitable carbohydrates are aldehyde or ketone compounds containing two or more hydroxyl groups. The carbohydrates may be cyclic or linear and include, for example, aldoses, ketoses, amino sugars, alditols, inositols, aldonic acids, uronic acids, or aldaric acids, or combinations thereof. The carbohydrate may also be a mono-, a di-, or poly-, carbohydrate, such as for example, a disaccharide or polysaccharide. Suitable carbohydrates include for example, glyceraldehydes, arabinose, lyxose, pentose, ribose, xylose, galactose, glucose, hexose, idose, mannose, talose, heptose, glucose, fructose, gluconic acid, sorbitol, lactose, mannitol, methyl α-glucopyranoside, maltose, isoascorbic acid, ascorbic acid, lactone, sorbose, glucaric acid, erythrose, threose, arabinose, allose, altrose, gulose, idose, talose, erythrulose, ribulose, xylulose, psicose, tagatose, glucuronic acid, gluconic acid, glucaric acid, galacturonic acid, mannuronic acid, glucosamine, galactosamine, sucrose, trehalose or neuraminic acid, or derivatives thereof. Suitable polycarbohydrates include, for example, arabinans, fructans, fucans, galactans, galacturonans, glucans, mannans, xylans (such as, for example, inulin), levan, fucoidan, carrageenan, galactocarolose, pectins, pectic acids, amylose, pullulan, glycogen, amylopectin, cellulose, dextran, pustulan, chitin, agarose, keratin, chondroitin, dermatan, hyaluronic acid, alginic acid, xanthin gum, or starch. Among particularly useful carbohydrates are sucrose, glucose, lactose, trehalose, and combinations thereof. Sucrose is a particularly useful cryoprotectant.

Preferably, the cryoprotectant of the present invention is a carbohydrate or sugar alcohol, which may be a polyhydric alcohol. Polyhydric compounds are compounds that contain more than one hydroxyl group. Preferably, the polyhydric compounds are linear. Suitable polyhydric compounds include, for example, glycols such as ethylene glycol, polyethylene glycol, and polypropylene glycol, glycerol, or pentaerythritol, or combinations thereof. In some preferred embodiments, the cryoprotectant agent is sucrose, trehalose, mannitol, or sorbitol. In another embodiment, the cryoprotectant is at a concentration of about 1.5% to about 6% by weight. Preferably, the cryoprotectant is sucrose at a concentration of about 5%.

It has been found to be desirable to add a surfactant to the pre-lyophilized formulation. Alternatively, or in addition, the surfactant may be added to the lyophilized formulation and/or the reconstituted formulation. Exemplary surfactants include nonionic surfactants such as polysorbates (e.g., polysorbates 20 or 80); poloxamers (e.g., poloxamer 188); Triton; sodium dodecyl sulfate (SDS); sodium laurel sulfate; sodium octyl glycoside; lauryl-, myristyl-, linoleyl-, or stearyl-sulfobetaine; lauryl-, myristyl-, linoleyl- or stearyl-sarcosine; linoleyl-, myristyl-, or cetyl-betaine; lauroamidopropyl-, cocamidopropyl-, linoleamidopropyl-, myristamidopropyl-, palnidopropyl-, or isostearamidopropyl-betaine (e.g., lauroamidopropyl); myristamidopropyl-, palmidopropyl-, or isostearamidopropyl-dimethylamine; sodium methyl cocoyl-, or disodium methyl oleyl-taurate; and the MONAQUAT™ series (Mona Industries, Inc., Paterson, N.J.), polyethyl glycol, polypropyl glycol, and copolymers of ethylene and propylene glycol (e.g., Pluronics or PF68), and Tween 80. The surfactant, in one embodiment, is at a concentration of about 0.005% to about 0.05% by weight. In a preferred embodiment, the surfactant is Polysorbate 80 at a concentration of 0.01% by weight or Tween 80 at a concentration of about 0.01% by weight.

A reconstituted formulation is one that has been prepared by dissolving a lyophilized antibody formulation in a diluent such that the antibody is dispersed in the reconstituted formulation. The reconstituted formulation in suitable for administration (e.g., parenteral administration) to a patient to be treated with the antibody of interest and, in certain embodiments of the invention, may be one which is suitable for subcutaneous administration.

By isotonic is meant that the formulation of interest has essentially the same osmotic pressure as human blood. Isotonic formulations will generally have an osmotic pressure from about 250 to 350 mOsm. Isotonicity can be measured using a vapor pressure or ice-freezing type osmometer, for example.

A lyoprotectant can also be added to the pre-lyophilized formulation. A lyoprotectant is a molecule which, when combined with a antibody of interest, significantly prevents or reduces chemical and/or physical instability of the antibody upon lyophilization and subsequent storage. Exemplary lyoprotectants include sugars such as sucrose or trehalose; an amino acid such as monosodium glutamate or histidine; a methylamine such as betaine; a lyotropic salt such as magnesium sulfate; a polyol such as trihydric or higher sugar alcohols, e.g., glycerin, erythritol, glycerol, arabitol, xylitol, sorbitol, and manmitol; propylene glycol; polyethylene glycol; Pluronics; and combinations thereof. The preferred lyoprotectant is a non-reducing sugar, such as trehalose or sucrose.

In preferred embodiments, the lyoprotectant is a non-reducing sugar such as sucrose or trehalose. The amount of lyoprotectant in the pre-lyophilized formulation is generally such that, upon reconstitution, the resulting formulation will be isotonic. However, hypertonic reconstituted formulations may also be suitable. In addition, the amount of lyoprotectant must not be too low such that an unacceptable amount of degradation/aggregation of the antibody occurs upon lyophilization. Where the lyoprotectant is a sugar (such as sucrose or trehalose), exemplary lyoprotectant concentrations in the pre-lyophilized formulation are from about 10 mM to about 400 mM, and preferably from about 30 mM to about 300 mM, and most preferably from about 50 mM to about 100 mM. The ratio of antibody to lyoprotectant is selected for each antibody and lyoprotectant combination. In the case of a sugar (e.g., sucrose or trehalose), as the lyoprotectant for generating an isotonic reconstituted formulation with a high antibody concentration, the molar ratio of lyoprotectant to antibody may be from about 100 to about 1500 moles lyoprotectant to 1 mole antibody, and preferably from about 200 to about 1000 moles of lyoprotectant to 1 mole antibody, and more preferably, from about 200 to about 600 moles of lyoprotectant to 1 mole antibody.

The lyoprotectant is added to the pre-lyophilized formulation in a lyoprotecting amount which means that, following lyophilization of the antibody in the presence of the lyoprotecting amount of the lyoprotectant, the antibody essentially retains its physical and chemical stability and integrity upon lyophilization and storage.

The diluent of interest herein is one that is pharmaceutically acceptable (safe and non-toxic for administration to a human) and is useful for the preparation of a reconstituted formulation. Exemplary diluents include sterile water, bacteriostatic water for injection (BWFI), a pH buffered solution (e.g. phosphate-buffered saline), sterile saline solution, Ringers solution or dextrose solution.

A preservative is a compound that can be added to the diluent to essentially reduce bacterial action in the reconstituted formulation, thus facilitating the production of a multi-use reconstituted formulation, for example. Examples of potential preservatives include octadecyldimethylbenzyl ammonium chloride, hexamethonium chloride, benzalkonium chloride (a mixture of alkylbenzyldimethylammonium chlorides in which the alkyl groups are long-chain compounds), and benzethonium chloride. Other types of preservatives include aromatic alcohols such as phenol, butyl and benzyl alcohol, allyl parabens such as methyl or propyl paraben, catechol, resorcinol, cyclohexanol, 3-pentanol, and m-cresol. The most preferred preservative herein is benzyl alcohol.

A bulking agent is a compound that adds mass to the lyophilized mixture and contributes to the physical structure of the lyophilized cake (e.g., facilitates the production of an essentially uniform lyophilized cake which maintains an open pore structure). Exemplary bulking agents include mannitol, glycine, polyethylene glycol and xorbitol.

In some instances, a mixture of the lyoprotectant (such as sucrose or trehalose) and a bulking agent (e.g., mannitol or glycine) is used in the preparation of the pre-lyophilization formulation. The bulking agent may allow for the production of a uniform lyophilized cake without excessive pockets therein. Thus, a bulking agent can also be added prior to lyophilization. Suitable bulking agent can have a concentration of about 0.5 to about 1.5% by weight. Preferably, the bulking agent is Dextran 40 at a concentration of 0.9% by weight or hydroxyethyl starch 40 at a concentration of 0.9% by weight.

Other pharmaceutically acceptable carriers, excipients or stabilizers such as those described in Remingtons Pharmaceutical Sciences 16th edition, Osol, A. Ed. (1980) may be included in the pre-lyophilized formulation (and/or the lyophilized formulation and/or the reconstituted formulation) provided that they do not adversely affect the desired characteristics of the formulation. Acceptable carriers, excipients or stabilizers are nontoxic to recipients at the dosages and concentrations employed and include additional buffering agents; preservatives; co-solvents; antioxidants including ascorbic acid and methionine; chelating agents such as EDTA; metal complexes (e.g., Zn-antibody complexes); biodegradable polymers such as polyesters; and/or salt-forming counterions such as sodium.

The formulations to be used for in vivo administration must be sterile. This is readily accomplished by filtration through sterile filtration membranes, prior to, or following, lyophilization and reconstitution. Alternatively, sterility of the entire mixture may be accomplished by autoclaving the ingredients, except for antibody, at about 120° C. for about 30 minutes, for example.

After preparation of the antibody of interest, a pre-lyophilized formulation is produced. The amount of antibody present in the pre-lyophilized formulation is determined taking into account the desired dose volumes, mode(s) of administration. The antibody is generally present in solution. For example, the antibody may be present in a pH-buffered solution. Exemplary buffers include histidine, phosphate, Tris, citrate, succinate and other organic acids. In one embodiment, the conjugate is at a concentration of about 0.5 mg/mL to about 2 mg/mL and, preferably, a concentration of 1 mg/mL. In one embodiment, the buffering agent is at a concentration of about 5 mM to about 50 mM. In a preferred embodiment, the buffering agent is Tris at a concentration of about 20 mM. Prior to lyophilization, the pH can be any suitable pH, for example, from about 7.8 to about 8.2 and, preferably, about 8.0.

The electrolyte in another embodiment of the present formulation is at a concentration of about 5 mM to about 100 mM. Any suitable electrolyte can be used, such as sodium, potassium, calcium, magnesium, chloride, phosphate, and bicarbonate, for example. Preferably, the electrolyte is a sodium or potassium salt and, more preferably, the electrolyte is NaCl at a concentration of about 10 mM.

After the antibody, lyoprotectant and other optional components are mixed together, the formulation is lyophilized. Many different freeze-dryers are available for this purpose such as Hull50.™. (Hull, USA) or GT20.™. (Leybold-Heraeus, Germany) freeze-dryers. Freeze-drying is accomplished by freezing the formulation and subsequently subliming ice from the frozen content at a temperature suitable for primary drying. Under this condition, the product temperature is below the eutectic point or the collapse temperature of the formulation. Typically, the shelf temperature for the primary drying will range from about −30 to 25° C. (provided the product remains frozen during primary drying) at a suitable pressure, ranging typically from about 50 to 250 mTorr. The formulation, size and type of the container holding the sample (e.g., glass vial) and the volume of liquid will mainly dictate the time required for drying, which can range from a few hours to several days (e.g., 40-60 hrs). A secondary drying stage may be carried out at about 0-40° C., depending primarily on the type and size of container and the type of antibody employed. However, it was found herein that a secondary drying step may not be necessary. For example, the shelf temperature throughout the entire water removal phase of lyophilization may be from about 15-30° C. (e.g., about 20° C.). The time and pressure required for secondary drying will be that which produces a suitable lyophilized cake, dependent, e.g., on the temperature and other parameters. The secondary drying time is dictated by the desired residual moisture level in the product and typically takes at least about 5 hours (e.g., 10-15 hours). The pressure may be the same as that employed during the primary drying step. Freeze-drying conditions can be varied depending on the formulation and vial size.

Lyophilization according to the present invention can comprise freezing the solution at a temperature of about −35° to about −50° C.; initially drying the frozen solution at a primary drying pressure of 20 to 80 microns at a shelf-temperature of about −10° to −40° C. for 24 to 78 hours; and secondarily drying the freeze-dried product at a secondary drying pressure of 20 to 80 microns at a shelf temperature of about +10° to +30° C. for 15 to 30 hours. Freezing can be carried out at −45° C., with the initial freeze drying at a primary drying pressure of 60 microns and a shelf temperature of −30° C. for 60 hours and with the secondary drying step at a drying pressure 60 microns and a shelf temperature of +25° C. for 24 hours.

It may be desirable to lyophilize the antibody formulation in the container in which reconstitution of the antibody is to be carried out in order to avoid a transfer step. The container in this instance may, for example, be a 3, 5, 10, 20, 50 or 100 cc vial.

As a general proposition, lyophilization will result in a lyophilized formulation in which the moisture content thereof is less than about 5%, and preferably less than about 3%.

At the desired stage, typically when it is time to administer the antibody to the patient, the lyophilized formulation may be reconstituted with a diluent such that the antibody concentration in the reconstituted formulation is at least 50 mg/mL, for example, from about 50 mg/mL to about 400 mg/mL, more preferably from about 80 mg/mL to about 300 mg/mL, and most preferably from about 90 mg/mL to about 150 mg/mL. Such high antibody concentrations in the reconstituted formulation are considered to be particularly useful where subcutaneous delivery of the reconstituted formulation is intended. However, for other routes of administration, such as intravenous administration, lower concentrations of the antibody in the reconstituted formulation may be desired (for example, from about 5-50 mg/mL, or from about 10-40 mg/mL antibody in the reconstituted formulation). In certain embodiments, the antibody concentration in the reconstituted formulation is significantly higher than that in the pre-lyophilized formulation. For example, the antibody concentration in the reconstituted formulation may be about 2-40 times, preferably 3-10 times and most preferably 3-6 times (e.g., at least three fold or at least four fold) that of the pre-lyophilized formulation.

Reconstitution generally takes place at a temperature of about 25° C. to ensure complete hydration, although other temperatures may be employed as desired. The time required for reconstitution will depend, e.g., on the type of diluent, amount of excipient(s) and antibody. Exemplary diluents include sterile water, bacteriostatic water for injection (BWFI), a pH buffered solution (e.g., phosphate-buffered saline), sterile saline solution, Ringers solution or dextrose solution. The diluent optionally contains a preservative. Exemplary preservatives have been described above, with aromatic alcohols such as benzyl or phenol alcohol being the preferred preservatives. The amount of preservative employed is determined by assessing different preservative concentrations for compatibility with the antibody and preservative efficacy testing. For example, if the preservative is an aromatic alcohol (such as benzyl alcohol), it can be present in an amount from about 0.1-2.0% and preferably from about 0.5-1.5%, but most preferably about 1.0-1.2%. Preferably, the reconstituted formulation has less than 6000 particles per vial which are >10 μm size.

The reconstituted formulation is administered to a human in need of treatment with the antibody, in accord with known methods, such as intravenous administration as a bolus or by continuous infusion over a period of time, by intramuscular, intraperitoneal, intracerebrospinal, subcutaneous, intra-articular, intrasynovial, intrathecal, oral, topical, or inhalation routes.

An article of manufacture is provided which contains the lyophilized formulation of the present invention and provides instructions for its reconstitution and/or use. This article of manufacture or kit has (i) a container which holds the compositions/formulations of the present invention; and (ii) instructions for reconstituting the lyophilized formulation with a diluent to a conjugate concentration in the reconstituted formulation within the range from 0.5 mg/mL to 5 mg/mL. Suitable containers include, for example, bottles, vials (e.g., dual chamber vials), syringes (such as dual chamber syringes) and test tubes. The container may be formed from a variety of materials such as glass or plastic. The container holds the lyophilized formulation and the label on, or associated with, the container may indicate directions for reconstitution and/or use. For example, the label may indicate that the lyophilized formulation is reconstituted to antibody concentrations as described above. The label may further indicate that the formulation is useful or intended for subcutaneous administration. The container holding the formulation may be a multi-use vial, which allows for repeat administrations (e.g., from 2-6 administrations) of the reconstituted formulation. The article of manufacture may further comprise a second container comprising a suitable diluent (e.g., BWFI). Upon mixing of the diluent and the lyophilized formulation, the final antibody concentration in the reconstituted formulation will generally be at least 50 mg/mL. The article of manufacture may further include other materials desirable from a commercial and user standpoint, including other buffers, diluents, filters, needles, syringes, and package inserts with instructions for use.

Once formulated, the compositions of the invention can be administered directly to the subject. The subjects to be treated can be animals. However, it is preferred that the compositions are adapted for administration to human subjects.

The compositions of the present invention may be administered by any number of routes including, but not limited to, oral, intravenous, intramuscular, intra-arterial, intramedullarly, intrathecal, intraventricular, transdermal, transcutaneous (see PCT Publication No.: WO98/20734), subcutaneous, intraperitoneal, intranasal, enteral, topical, sublingual, intravaginal or rectal routes. Hyposprays may also be used to administer the compositions of the invention. Typically, the compositions may be prepared as injectables, either as liquid solutions or suspensions. Solid forms suitable for solution in, or suspension in, liquid vehicles prior to injection may also be prepared.

### EXAMPLES

General Materials and Methods

Carcinoma Cells

Human carcinoma cell lines expressing varying levels of Lewis Y antigen on the surface were selected. These included cell lines that had high expression of the Lewis Y antigen (L2987 lung carcinoma, N87 gastric carcinoma, A431/LeY epidermoid carcinoma, AGS colon carcinoma, and LS174T colon carcinoma), cell lines that had low expression of the Lewis Y antigen (LOVO colon carcinoma and LNCaP prostate carcinoma), and cell lines that had very low or no expression of the Lewis Y antigen (PC3MM2 prostate carcinoma, and A431 epidermoid carcinoma). The Lewis Y expression status of the carcinoma cell lines used was confirmed by flow cytometry. Examples of the cell lines used are as follows.
DLD-1 (CCL-221), HCT8S11, HCT8S11/R1 and LOVO (CCL-229) are colon carcinoma cell lines that display Le^{y} antigen on the cell membrane.
NCI-H157 (CRL-5802), NCI-H358 (CRL-5807) and A549 (CCL-159) are lung carcinoma cell lines. Of these three cell lines, NCI-H358 displayed detectable levels of Le^{y} on the cell surface.
Both gastric carcinomas N87 (CRL-5822) and AGS (CRL-1739) express Le^{y}.
A431 (CRL-1555) and A431/Le^{y} are epidermoid (cervical) carcinoma cells. Only the latter variant expresses Le^{y}.
MDA-MB435 (Le^{y-}) and MDA-MB-361 (Le^{y+}) were used as models of breast carcinoma cells.
PC3-MM2 (Le^{y-}) and LNCaP (Le^{y+}, CRL-1740) were derived from prostate carcinomas.

All the cell lines, except HCT8S11, HCT8S11/R1, MDA-MB435, PC3-MM2 and A431/Le^{y}, were purchased from the American Type Culture Collection (ATCC). Cell lines obtained from ATCC were maintained in culture medium as specified in the ATCC-catalogue. HCT8S11 and HCT8S11/R1 are a gift from Dr. M. Mareel (University Hospital, Ghent, Belgium). These cells were grown in RPMI 1640 supplemented with 10% v/v fetal bovine serum (FBS), 1 mM sodium pyruvate, 100 μg/ml streptomycin and 100 U/ml penicillin (hereafter called pen/strep). MDA-MB435 and PC3-MM2 were obtained from Dr. I. Fidler (MD Anderson, Tex.). These cells were cultured in minimum essential medium supplemented with 10% v/v FBS, 2 mM glutamine, 1 mM sodium pyruvate, 0.2 mM non-essential amino acids, 2% MEM vitamin solution, and pen/strep. A431/Le^{y} was provided by Ludwig Institute for Cancer Research (Melbourne, Australia). They were cultured in DMEM/F12 supplemented with 10% FBS, 2 mM glutamine and pen/strep.

Antibodies

RITUXAN® (rituximab; IDEC Pharmaceuticals Corporation and Genentech, San Diego and San Francisco, Calif.) is a chimeric antibody that combines the murine heavy and light chain variable regions with the human IgG1k constant regions. The antibody recognizes the B-lymphocyte marker CD20. For FACS-analysis, human IgG (huIgG, Zymed, San Francisco, Calif.) and FITC-labeled goat anti-huIgG (FITC/α-huIgG, Zymed, San Francisco, Calif.) were used as control antibody and as secondary antibody, respectively. RITUXAN was used as a negative control because FACS analyses showed that the antigen recognized by RITUXAN (CD20) was present in trace amounts on the surface of the cells used in the described experiments. A calicheamicin-conjugate of RITUXAN controlled for the carrier function of immunoglobulins and the hydrolytic release of calicheamicin.

MYLOTARG® (gemtuzumab ozogamicin, also referred to as CMA-676 or simply CMA) is a calicheamicin conjugate (Wyeth, Madison, N.J.). A batch with an average amount of 35 ug calicheamicin conjugated to 1 mg antibody was used. The antibody portion of CMA or CMA-676 is specific for CD33, which is a leukocyte differentiation antigen expressed by multipotential hematopoietic stem cells and acute myeloid leukemic cells. None of the cells used in any of the described experiments expressed significant levels of CD33. Indeed, FACS analysis showed that the amount of CMA bound to these cell lines was similar to the amount of control huIgG1 demonstrating a lack of CD33 expression. The highest binding of CMA was determined in PC3MM2 cells (re MCF=2.3). Therefore, CMA also controls for the efficacy of released CM without antigen targeting of the conjugate.

Plasmon Resonance Analysis (Biacore)

The Lewis-BSA conjugates (i.e., H type I-, H type II-, Sialyl Le^{a}-, Sialyl Le^{x}-, Sulfo Le^{a}-, Sulfo Le^{x}-, Le^{a}-, Le^{b}-, Le^{x} and Le^{y}-BSA) were purchased from Alberta Research Council (Edmonton, Alberta, Canada). The antigen/BSA loading was between 20 to 42 mole antigen/mole of BSA. Each antigen was immobilized to the surface of a CM5 biosensor chip at a density of 4,000 to 9,000 RU. The chip was activated by the coupling reagent EDC/NHS [1-Ethyl-3-(3-dimethylaminopropyl)-carbodiimide-HCl]/[N-Hydroxysuccinimide] at a flow rate of 5 μl/min for 6 minutes, followed by the addition of the Lewis-BSA antigens at 5 μl/min for 6 minutes at a concentration of 50 μg/ml in 10 mM sodium acetate buffer pH 4.5. The Sulfo-Lewis and Sialyl-Lewis-BSA conjugates were coupled at pH 4.0. Surplus binding sites were blocked with 1 M ethanolamine-HCl pH 8.5 at 5 μl/min for 6 minutes. Binding specificity analysis was performed in HBS-EP buffer (10 mM HEPES, 150 mM NaCl, 3 mM EDTA, 50 ppm polysorbate 20) at a flow rate of 20 μl/min. Hu3S193 was injected for 3 minutes at 6.67 nM or 50 nM. The amount of antibody that remained bound after a 30 second wash with HBS-EP buffer was measured. The antigenic surface was regenerated by 10 mM NaOH, 200 mM NaCl for 1 minute at 20 μl/min., to re-establish a baseline.

For kinetic analysis, antibody was used in concentrations of 1 to 16 nM. The density of Le^{y}-BSA was 9,000. Association and dissociation were measured in HBS-EP buffer during 3 and 15 minutes at 30 μl/min.

FACS-Analysis

The presence of Le^{y} on a series of human tumor cell lines was evaluated by FACS analysis. Aliquots of 10⁵ cells were suspended in 100 μl phosphate buffered saline supplemented with 1% v/v bovine serum albumin (PBS/BSA). The cells were then incubated at 4° C. for 30 minutes in various concentrations of primary antibody, hu3S193 or G193, hu3S193, or CM-conjugates. Binding of the primary antibody to the cells was revealed by FITC labeled/α-huIgG.

The MCF (mean channel fluorescence) values are the average fluorescent intensity of cell populations following binding with the primary antibodies (huIgG and hu3S193) and consecutive staining with a fluorescent-labeled secondary antibody. HuIgG is a negative control. The MCF is directly proportional to the number of bound primary antibody molecules. The majority (8 out of 13) of the investigated cell lines expressed Le^{y} as seen by at least a 10-fold (relative MCF, reMCF) increase of the MCF after hu3S193 binding over the MCF of the negative control. Examples of cell lines with high expression of Le^{y} were found in each histiotypic tumor category. All tumor cells of colorectal and gastric origin were Le^{y}-positive.

ED₅₀ of Anti-Lewis Y Antibodies Conjugated to Calicheamicin

A vital dye (MTS) staining was used to determine the number of surviving cells following exposure to various treatments. MTS (non-radioactive cell proliferation assay kit) was purchased from Promega (Madison, Wis.) and used according to the manufacturer's specifications. For each cell line, a calibration curve (cell number versus optical density after 2 h) was established to estimate an appropriate initial seeding density. Cells were then seeded in 96-multiwell dishes at a density of 750 to 5,000 cells per well. Immediately after seeding, the cells were exposed to various concentrations (0, 0.01, 0.05, 0.1, 1, 10, 100 and 500 ng calicheamicin equivalents/ml) of CMA, hu3S193-AcBut-CM, or CM, or to PBS. Each well received 10 μl of 100× drug solution. Following determination of the number of cells surviving 96 h of drug-exposure, the ED₅₀ was calculated based on the logistic regression parameters derived from the dose-response curves. The ED₅₀ was defined as the molar concentration of drug (CM) that caused a 50% reduction of the cell number after 96 hours exposure to the drug. It should be noted that a calicheamicin equivalent (cal. eq.) is the concentration of CM given either as a pure substance or as a conjugate. Dependent on the amount of CM bound to the antibody (antibody drug loading), a calicheamicin equivalent of different conjugates can imply different protein concentrations.

### Example 1

### Generation of Anti-Lewis Y Antibodies

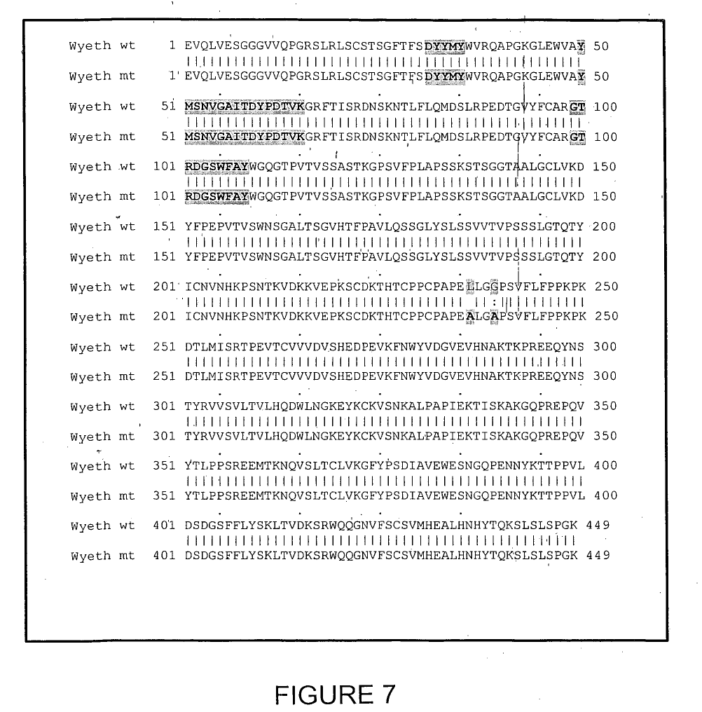

Wild-type (hu3S193) and mutant (G193) anti-Lewis Y antibodies were generated. The murine 3S193 mAb was generated by immunization of BALB/c mice with human adenocarcinoma cells positive for the Lewis Y antigen. A humanized version of the 3S193 antibody was subsequently generated (hu3S193). Detailed specificity analysis demonstrated that hu3S193 was highly specific for Le^{y} (no binding to H-type 2 or type 1 antigens) and displayed only minimal cross-reactivity with the Le^{x} trisaccharide. The mutated IgG1 version of hu3S193 (G193) differs from hu3S193 in that it has two amino acid substitutions in its CH2 domain, namely: leucine (234) to alanine and glycine (237) to alanine. In addition to the above two mutations, there were two additional conservative mutations (aspartic acid at position 358 to glutamic acid, and methionine at position 360 to leucine) corresponding to the Gmz allotype in the CH3 domain of IgG1. Thus, the humanized mutant IgG1 anti-Lewis Y antibody differed from hu3S193 at 4 residues within the Fc region; L236A, G239A, D358E, and M360L. This mutant IgG1 form of anti-Lewis Y antibody was named G193, expressed in Chinese hamster ovary cells, and was used to create CMD-193. FIG. 7 provides a comparison of the amino acid sequences of the mature secreted heavy chains of the two antibodies. In this figure, the mutant residues are bolded and highlighted and the CDRs are bolded and shaded.

Cells and Culturing Conditions

Hybridoma cells that expressed hu3S193 antibody were obtained from Ludwig Institute for Cancer Research. Hu3S193 is humanized anti-Le^{y} antibody (IgG1) derived from the mouse monoclonal antibody MuS193, which has been engineered so that only the complementary determining regions are from murine origin.

The cell line is a cholesterol dependent cell line and requires the addition of cholesterol in the Hyclone HyQ-CCM®1 growth medium (Hyclone Labs, Logan, Utah). Because cholesterol is not water-soluble, the medium was supplemented with 0.2% ExCyte VLE (Miles Pentex, Kankake, Ill.). Cells were maintained at 37° C. in 5% CO₂. COS-7 cells were purchased from ATCC (Rockville, Md.) and maintained in Dulbeccos Modified Eagle Medium (DMEM), supplemented with 10% fetal bovine serum (FBS) and 2 mM glutamine, 100 U/ml penicillin, 100 μg/ml streptomycin (hereafter called pen/strep).

PA-DUKX 153.8 cells are deficient in production of dihydrofolate reductase (dhfr). These cells were maintained in Minimum Essential Medium (MEM-α, Gibco BRL, Grand Island, N.Y.) supplemented with 10 μg/ml adenosine, deoxyadenosine and thymidine (Sigma, St. Louis, Mo.), 10% FBS, 20 mM HEPES, 0.1% Sodium Bicarbonate, 2 mM glutamine and pen/strep. After transfection cells were grown in the absence of nucleotides and maintained with 1 mg/ml of G418 (Gibco) and 250 nM methotrexate (Sigma) as selection markers.

Vectors

To create the heavy and light chain constructs of the wild-type (hu3S193) and mutant (G193) anti-Lewis Y antibodies, the following vectors were used: PED6_HC_IgG1, PED6_HC_mIgG 1 and pED6_LC kappa vectors. PED6_HC_mIgG 1 vector contains the template for a mutated CH2 domain (the vector encodes Alanine at both position 234, replacing a Leucine, and position 237, replacing a Glycine). DNA of the variable region of the light chain of hu3S193 was ligated between the BssH II and the Pacl restriction sites of the pED6_LC k expression vector. DNA of the variable region of the heavy chain of hu3S193 was ligated between the BssH II and the Sal I restriction sites of the pED6_HCIgG1 expression vector. The vector pED6_Hc_mIgG1 was used to generate the heavy chain of G193. This vector differed from pED6_HC_IgG1 in the sequence of its domain. Expression of pED6_HC_mIgG1 yields a heavy chain with Alanine substituting for Leucine (234) and Glycine (237)

DNA encoding the variable and constant region of the light chain of G193 or hu3S193 was cut out from the pED6_LCₖ plasmid ligated between the PpUM and EcoR I restriction sites of pMEN2 vector. DNA encoding the variable and constant region of the heavy chain of G193 or hu3S193 was cut out from the pED6_HC_IgG1 or pED6_HC_mIgG1 vectors and inserted between the Bgl II and Xba I restriction sites of the pTDMEDL vector.

Extraction and Cloning

RNA was extracted from hu3S193-producing cells by means of an RNAzolB kit (RNAzol B, TEL-TEST, Inc., Friendswood, Tex.) according to the manufacturer's instructions. Using a kit (Stratagene, La Jolla, Calif.), the extracted RNA was transcribed into single stranded cDNA. Ten μg of total RNA was mixed with an oligo dT primer. This reaction mixture was heated to 65° C. for 5 minutes and slowly cooled to 22° C. First strand cDNA was synthesized in a mixture containing 5 μl of 10-fold concentrated first strand buffer, 5 μl DTT, 1 μl of RNAse block, 2 μl DNTPs (1.25 mM) and 1 μl of MMLV reverse transcriptase (20 U/μl) in a total volume of 50 μl. The components were gently mixed and incubated at 37° C. for 1 hour. This cDNA was used to amplify both VH and VK of hu3S193. The following primers were used for the polymerase chain (PCR) reaction:

(SEQ ID NO:1)

Hu3S193 VH UP (BssH II)
GCTTGGCGCGCACTCC GAG GTC CAA CTG GTG GAG AGC GGT
GGA GGT GTT

(SEQ ID NO:2)

Hu3S193 VH DN (Sal I)
GCGACGTCGACAGGACTCACC TGA GGA GAC GGT GAC CGG GGT
CCC TTG GCC CCA GTA AGC AAA

(SEQ ID NO:3)

Hu3S193 VK UP (BssH II)
GCTTGGCGCGCACTCC GAC ATC CAG ATG ACC GAG AGC CCA
AGC AGC CTG A

(SEQ ID NQ:4)

Hu3S193 VK DN (Pac I)
GCCCTTAATTAAGTTATTCTACTCACG TGT GAT TTG CAG CTT
GGT CCC TTG GCC GAA CGT GAA

The PCR reaction was carried out in a mixture of 5 μl of first strand cDNA, 100 ng of the sense and the antisense primer, 5 μl 10×PFU polymerase buffer, 500 μM MgCl₂, 1.25 mM DNTPs and 1 μl PFU enzyme (2 U/μl) in a total volume of 50 μl. The reaction consisted of 35 alternating cycles of denaturation (95° C.-1 min) and synthesis (72° C.-4 min) and 1 termination cycle (72° C.-7 min). The reaction product was analyzed by electrophoresis in 1% agarose. The PCR products were purified, and the heavy chain PCR product was digested with BssH II and Sal I and ligated into BssH II/Sal 1-digested pED6_HC_mIg1 or pED6_HC_Ig1 expression vectors to create the G193 and hu3S193 heavy chain constructs, respectively. Similarly light chain PCR product was digested with BssH II/Pac I and ligated in to BssH II/Pac I digested pED6_LC kappa expression vector to create the G193 light chain construct. The pED vectors were used to determine the expression of the antibody in a transient transfection experiment.

Further, pED vectors containing heavy and light chain of G193 or hu3S193 were subcloned in to pTDMEDL-DHFR/VH and pMEN2-NeoNK. To make these constructs, hu3S193 pED6_HC_mIgG1 VH (hu3S193 VH+CH1+mtCH2+CH3) and hu3S193 pED6_HC_IgG1 VH (hu3S193 VH+CHI+CH2+CH3) were digested with Bgl II and Xba I and ligated in to Bgl II/Xba I digested pTDMEDL vector to create the G193 VH/pTDMEDL-DHFR or hu3S193 VH/pTDMEDL-DHFR. Similarly pED6_LC kappa hu3S193 VK was digested with PpUM and EcoR I (hu3S193 VK+CK) and ligated into the PpUM and EcoR I digested pMEN2 vector to create the Hu3S193 VK/pMEN2-Neo. The sequence for G193 mAb is SEQ ID NO:13.

Ligation, Transformation, and Plasmid Purification

The digested products were ligated with T4 DNA ligase (Gibco) at 12° C. overnight and transformed into DH5α cells. Single colonies were inoculated into 2 ml LB cultures in the presence of 50 μg/ml ampicillin and grown at 37° C. overnight. Restriction mapping on miniprep DNAs confirmed the appropriate length of the inserts. Upon confirmation maxiprep DNA was made using a Qiagen-kit (Qiagen, Valencia, Calif.) according to the manufacturer's recommendation.

Sequencing of VH and VK DNA

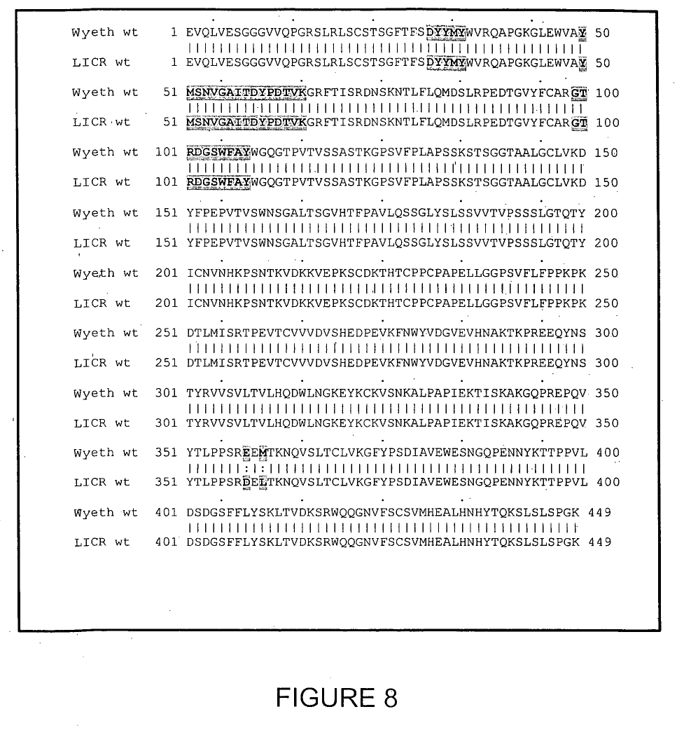

Maxiprep DNA was sent to DNA core facility for sequencing the variable heavy and light chain of hu3S193. The DNA sequence was determined as follows. A Qiagen 9600 robot (Qiagen) made the minipreps following the turbo prep method provided by the manufacturer. Five hundred ug of this miniprep DNA was mixed with 20 pM primer DNA in 13 μl H₂O. The DNA was then denatured by heating (98° C., 5 min) and cooling (4° C., 5 min). Eight μl Big Dye Terminators (ABI, Foster City, Calif.) was added to the denatured DNA. The mixture was heated to 98° C. and subjected to a series of 25 thermocycles (96° C., 20 s; 55° C., 20 s; 62° C., 120 s) and 20 thermocycles (96° C., 20 s; 60° C., 120 s). The reaction mixtures were filtered through Biosystems 96-well filtration plates (Edge, Gaithersburg, Md.) to remove excess dye terminators. The DNA fragments were then analyzed on a 3700 capillary array sequencer (ABI). The sequences of both heavy and light chain of G193 and hu3S193 are presented in FIGS. 7 and 8.

Transient Transfection of COS-7 Cells

Antibody expression was confirmed following transient transfection in COS-7 cells. One million COS-7 cells were plated on a 6 well dish. The following day, equimolar concentrations (a mixture of 1 μg of each) of either hu3S193 pED6_HC_mIgG1 VH and hu3S193 pED6_HC_mIgG1 VH or hu3S193 pED6_HC_IgG1 VH and hu3S193 pED6_HC_IgG1 VH were diluted in 250 μl serum-free DMEM. Also, 6 μl of 1 mg/ml Lipofectamine (Invitrogen) were also diluted in 250 μl serum-free DMEM. DNA and Lipofectamine were mixed and incubated for 15 minutes at room temperature. This mixture was added to the cells (cells were washed with serum free medium prior to the exposure of DNA-Lipofectamine complex). After incubation at 37° C. for 8 hours, fresh medium was added to the cells. Culture medium that was exposed to the cells for 48 hours was assayed for the presence of antibody by FACS and BIAcore analysis.

Stable Cell Lines

Following confirmation of antibody expression, stable lines expressing mutant (G193) and wild-type (hu3S193) anti-Lewis Y IgG1 antibodies were generated in PA-DUKX 153.8 cells as follows. Five million cells were plated in dishes with a diameter of 10 cm. After 16 h, an equimolar mixture (10 μg of each) of either G193 VH/pTDMEDL (clone #18) and VK/pMEN2 (clone #1) or the equivalent constructs for hu3S193 were diluted with 1.5 ml serum-free MEM-α. Sixty μl of Lipofectamine was also diluted with 1.5 ml serum-free MEM. DNA and Lipofectamine were mixed and incubated for 15 minutes at room temperature. This mixture was added to the cells that were then placed at 37° C. for 8 hours. After this period the mixture was replaced with 15 ml fresh growth medium. After 24 hours, cell cultures were passed at a 1:10 dilution into growth medium (without ribonucleosides and deoxyribonucleosides) containing 1 mg/ml of G418 and a step-wise increasing concentration of fresh Methotrexate (20, 40, 80, 100, 120, 160, 200 and 250 nM/ml). Colonies were picked and expanded. The conditioned culture media from these clones were analyzed by FACS, BIAcore and ELISA. Stable cell lines that expressed G193 or hu3S193 were then used for mass-production and purification of the antibody.

Effector Functions of G193

To determine the effector functional capabilities of G193 and its conjugate, CMD-193, both were examined using both N87 gastric carcinoma cells that had expression of the Lewis Y antigen and A431 epidermoid carcinoma cells that had very low or no expression of the Lewis Y antigen. Wild-type humanized IgG1 anti-Lewis Y antibody, hu3S193, was used as a positive control. This antibody has been shown to mediate both the ADCC and CDC activities. Freshly isolated human peripheral blood mononuclear cells (PBMNC) were used as the source of effector cells during the ADCC assays and freshly prepared human serum was used as a source of complement in CDC assays.

CDC activity of G193 and CMD-193 was evaluated using a fixed number of tumor cells cultured for 4 hr with different concentrations of anti Lewis Y antibodies in the presence of 1:100 dilution of fresh human serum as a source of complement. Lactate dehydrogenase activity released as a result of the lysis of tumor cells was measured. LDH activity release by a nonionic detergent was measured as a representation of total lysis. Similar evaluation was conducted with A431 cells expressing a high level of Lewis Y (Lewis Y⁺⁺⁺), i.e., high Lewis Y.

ADCC activity of G193 and CMD-193 was determined using a fixed number of tumor cells cultured for 4 hr with different concentrations of anti-Lewis Y antibody in the presence or absence of peripheral blood mononuclear cells used as effector cells at effector cell:target cell ratio of 50. Lactate dehydrogenase activity released as a result of the lysis of tumor cells was measured. LDH activity release by a nonionic detergent was measured as a representation of total lysis. Similar evaluation was conducted with Lewis Y⁺⁺⁺ N87 cells.

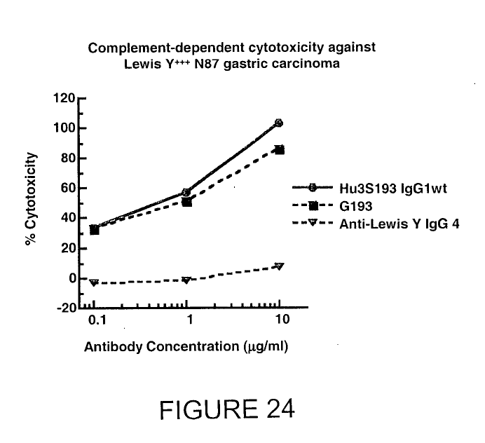

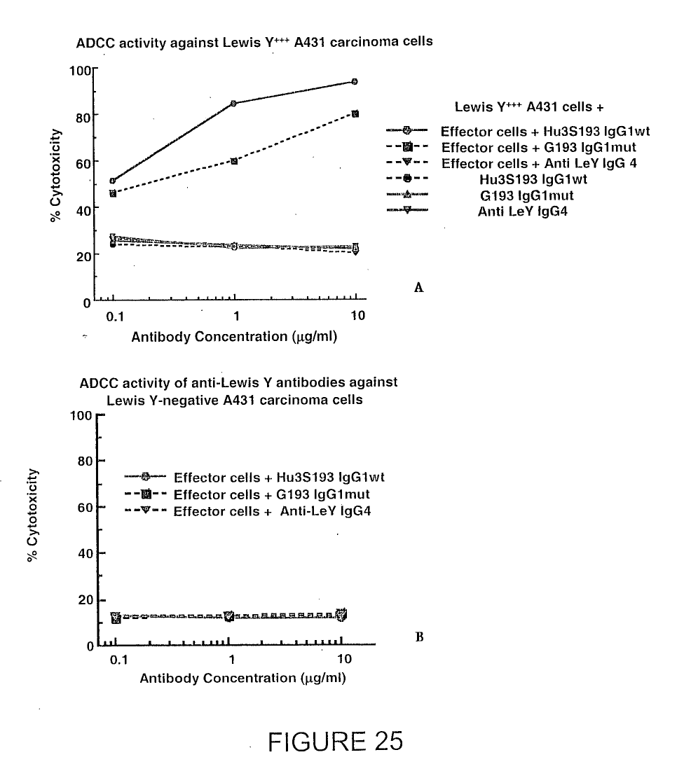

Both the wild-type IgG1 and the mutant IgG1 anti Lewis Y antibody were equally able to mediate both the ADCC and CDC activities against N87 carcinoma cells that had high expression of the Lewis Y antigen, as shown in FIGS. 24 and 25. Similar activity of either antibody was not observed against A431 cells that had very low or no expression of the Lewis Y antigen. In contrast, an IgG4 version of anti-Lewis Y antibody with VH and VK sequences identical to those of hu3S193 and G193 was incapable of promoting both ADCC and CDC activities. Human IgG4 isotype is known to be deficient in its ability to mediate ADCC and CDC, and consistent with this notion, the anti-Lewis Y IgG4 antibody is inactive in the ADCC and CDC assays.

These results suggest that the introduction of the L236A and G239A mutations in the Fc of G193 did not render G193 deficient in its effector functional capabilities. CMD-193 was also as effective as G193 in mediating CDC activity against N87 carcinoma cells that had high expression of the Lewis Y antigen. These results further indicate that the conjugation of G193 to calicheamicin does not alter the ability of G193 to mediate CDC activity. Thus, both G193 and CMD-193 are capable of mediating effector functional activities, and CMD 193 is an effector function-competent antibody conjugate.

### Example 2

### Conjugation of Anti-Lewis Y Antibodies to Calicheamicin

Antibodies were initially conjugated to calicheamicin (CM) as follows. The antibody at a protein concentration of approximately 10 mg/ml was adjusted to pH 8-8.5 with a high molarity non-nucleophilic buffer (1 M HEPES). Next, an excipient (sodium octanoate) that prevents protein aggregation was added at a final concentration of 0.1-0.2 M. Finally, 5% of the protein mass of activated calicheamicin derivative was added as a concentrated solution (10-20 mg/ml) in an organic solvent (ethanol or dimethylformamide). This reaction mixture was then incubated at 25-35° C. for 1 to 2 h. Progress of the reaction was monitored by SEC-HPLC. After completion of the reaction, the conjugate was separated from aggregated antibody and free calicheamicin on a preparative SEC column. The amount of CM per antibody of conjugate preparations that was used in the presented experiments ranged between 22 and 47 μg/mg and between 17 and 30 μg/mg for hu3S193-AcBut-CM and RITUXAN-AcBut-CM, respectively.

Optimizing Conjugation Conditions

In a typical conjugation reaction, humanized anti-Lewis Y antibody (hu3S193) was conjugated to NAc-gamma-calicheamicin-DMH-AcBut-OSu (calicheamicin derivative) where the target protein concentration was 10 mg/ml and the target calicheamicin derivative loading was 7.0 percent by weight of the protein. The target reaction pH was 8.2±0.2 and the target concentration of the other reaction components were as follows: 50 mM HEPBS, 10 mM sodium deoxycholate, and 9% v/v ethanol. The reaction was conducted at 33±2° C. for one hour. Results of the analysis of this typical reaction prior to purification were as follows: Protein: 9.92 mg/ml; Calicheamicin Loading: 70 mcg/mg; Aggregate: 1.9%; Unconjugated Protein (LCF): 0.81%.

The effect of various surfactant additives and their concentration on product yield and purity were tested to determine their effect on the production of conjugated monomer of hu3S193. The results are shown below in Table 2. Reactions were run where all variables were held constant except for the additive and its concentration. The conjugates produced from these reactions were analyzed for aggregate, LCF and protein recovery. Although several additives produced conjugates with either low aggregate or low LCF, only deoxycholate produced a conjugate with low aggregate, low LCF and high protein recovery.

TABLE 2

| Detergent | Aggregate | Unconjugated | Yield |
|---|---|---|---|
| (Concentration) | (%) | mAb (%) | (%) |
| Ethylene glycol (10%) | 16.7 | 16.3 | 36 |
| Tween-80 | 18.5 | 19.5 | 38 |
| Fusidic Acid (10 mM) | 9.9 | 25.2 | 47 |
| Fusidic Acid (20 mM) | 13.7 | 10.5 | 49 |
| Fusidic Acid (50 mM) | 25.6 | 5.2 | 46 |
| Octanoate (180 mM) | 9.8 | 26.4 | 37 |
| Octanoate (200 mM) | 24.2 | 10.1 | 38 |
| Octanoate (220 mM) | 36.8 | 8.4 | 33 |
| Decanoate (20 mM) | 24.9 | 64.3 | 23 |
| Decanoate (50 mM) | 71.9 | 0 | 9 |
| Octyl Sulfate (75 mM) | 5 | 24 | 78 |
| Octyl Sulfate (100 mM) | 42 | 6 | 38 |
| Octyl Sulfate (150 mM) | 74 | 1 | ND |
| Decyl Sulfate (20 mM) | 47 | 6 | 35 |
| Decyl Sulfate (30 mM) | 69 | 6 | 13 |
| Decyl Sulfate (40 mM) | 68 | 7 | 7 |
| t-Butyl Ammonium | 1 | 58 | 85 |
| Chloride (150 mM) | | | |
| t-Butyl Ammonium | 5 | 35 | 74 |
| Chloride (250 mM) | | | |
| Deoxycorticosterone- | 1 | 100 | 99 |
| hemis (4 mM) | | | |
| Deoxycorticosterone- | 0 | 100 | 94 |
| hemis (10 mM) | | | |
| Deoxycorticosterone- | 0 | 59 | 41 |
| hemis (20 mM) | | | |
| Hydrocortisone- | 1 | 57 | 88 |
| hemis (20 mM) | | | |
| Hydrocortisone- | 1 | 26 | 85 |
| hemis (40 mM) | | | |
| Hydrocortisone- | 2 | 13 | 81 |
| hemis (80 mM) | | | |
| Benzoate (100 mM) | 0 | 72 | 100 |
| Benzoate (200 mM) | 0 | 66 | 92 |
| Benzoate (400 mM) | 1 | 50 | 82 |
| Benzoate (1 M) | 11 | 31 | 83 |
| Napthoic Acid (40 mM) | 4 | 52 | 98 |
| Napthoic Acid (100 mM) | 5 | ND | 64 |
| 4-Phenyl Butyric Acid | 2 | 52 | 100 |
| (167 mM) | | | |
| Dihydroxyl Phenyl | 0 | 67 | 100 |
| Acetic Acid (175 mM) | | | |
| Deoxycholate (15 mM) | 3.4 (1-6.2) | 2.3 (0.7-3.3) | 73 (63-80) |
| Glycodeoxycholic Acid | 4.1 | 3.12 | 72.8 |
| Taurodeoxycholic Acid | 4.6 | 4.5 | 71.7 |
| Cholic Acid | 9.8 | 28.4 | 67.9 |
| Gycocholic Acid | 13.3 | 33.9 | 60.7 |
| Taurocholic Acid | 15 | 35.1 | 64.3 |

Octanoate is the standard catalyst used in the CMA-676 conjugation reaction, while decanoate is a standard catalyst used in the CMC-544 conjugation reaction. The deoxycholate results are the average of 5 reactions with the range in parentheses. Other members of the bile acid family of detergents were tested and gave similar results.

The percent aggregate and percent free protein at the end of the conjugation reaction was determined for various IgG1 and IgG4 antibodies using octanoate, decanoate, and deoxycholate. The IgG1 antibodies tested were G193 and a control antibody, mAb 01, while the IgG4 antibodies tested were G193 with an IgG4 constant region (G193-IgG4), mAb 676 from the CMA-676 conjugate, mAb G544 from the CMC-544 conjugate, and a control antibody, mAb O₂. As shown below in Table 3, conjugation of IgG1 antibodies in the presence of deoxycholate resulted in low percent aggregate and low percent unconjugated protein (or LCF), while conjugation in the presence of octanoate or decanoate resulted in either high percent aggregate or high percent unconjugated protein. This is in contrast to IgG4 antibodies, which had low percent aggregate and low percent unconjugated protein when conjugated in the presence of either decanoate or deoxycholate.

TABLE 3

| | Octanoate | | Decanoate | | Deoxycholate | |
|---|---|---|---|---|---|---|
| | | Uncon- | | Uncon- | | Uncon- |
| | | jugated | | jugated | | jugated |
| | Aggregate | Protein | Aggregate | Protein | Aggregate | Protein |
| mAb | (%) | (%) | (%) | (%) | (%) | (%) |
| G193* | 5.68 | 39.7 | 15.0 | 2.25 | 2.91 | 1.08 |
| G193- | 4.48 | 38.7 | 28.03 | 0.13 | 4.33 | 3 |
| IgG4 | | | | | | |
| 676 | 9.22 | 48.8 | 5.29 | 34.4 | 3.36 | 29.4 |
| 01 | 6.03 | 36.5 | 6.92 | 14.92 | 6.55 | 2.3 |
| 02 | 5.47 | 41.16 | 5.03 | 0.07 | 3.44 | 3.44 |
| G544 | 3.75 | 37.55 | 2.34 | 3.29 | 3.57 | 3.34 |
| IgG1 | 5.86 | 38.1 | 10.96 | 8.59 | 4.73 | 1.69 |
| Avg | | | | | | |
| IgG4 | 5.73 | 41.55 | 10.17 | 9.47 | 3.68 | 9.80 |
| Avg | | | | | | |

*average of three runs

Different Drug Loadings of CMD-193

Different drug loadings of calicheamicin per anti-Lewis Y antibody (G193) were evaluated. CMD-193 preparations with the drug loadings of 30, 60 or 90 mg of NAc-gamma calicheamicin DMH per milligram of G193 antibody protein were generated and administered IP Q4Dx3 at a dose of 160 mg of calicheamicin equivalents per kilogram in N87 xenografted mice. The antitumor efficacy of CMD-193 was not impacted by the differences in the drug loading.

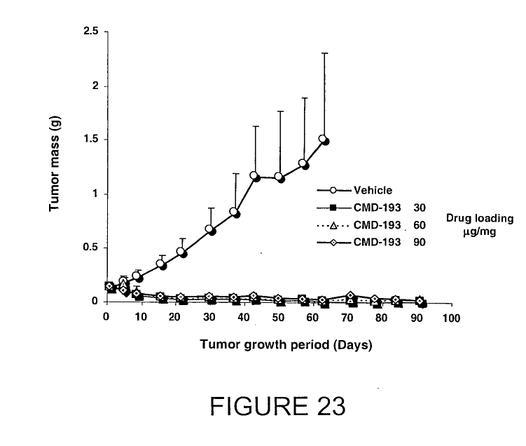

As shown in FIG. 23, the antitumor efficacy of CMD-193 with different calicheamicin loadings was essentially identical. Since the unconjugated targeting antibody G193 is ineffective in mediating antitumor activity, the entire antitumor efficacy of CMD-193 can be attributed to the targeted delivery calicheamicin to the tumor cells. These results also suggest that the degree of calicheamicin conjugation (loading) in the range of 30 to 90 μg/mg of CMD-193 does not impact its therapeutic outcome.

Chromatographic Purification

The starting material for the purification was a conjugation reaction mixture containing 9.92 mg/mL protein at a calicheamicin derivative loading of 70 μg/mg, with an aggregate content of 1.9% (area percent by HPLC), and an LCF content of 0.82% (area percent by HPLC). After the conjugation reaction was completed, the reaction mixture was diluted 10-fold by the addition of potassium phosphate solution to a final phosphate concentration of 0.6 M (pH 8.2). After mixing, this solution was filtered through 0.45-micron filters. The diluted solution was loaded on a Butyl Sepharose 4 Fast Flow column. The total amount of protein loaded on the column was 20 mg per ml bed volume. After a wash with 0.6 M potassium phosphate, the column was eluted using a step gradient from 0.6M to 4 mM potassium phosphate, pH 8.2 (alternatively, the column can be eluted with 20 mM Tris/25 mM NaCl). The fractions from the step gradient were pooled and the pool contained: Protein 8.3 mg/mL; Calicheamicin 69.3 mcg/mg; Aggregate 0.42%; LCF: 0.31%.

Buffer exchange was accomplished using ultrafiltration/diafiltration with a regenerated cellulose membrane. The conjugate was diafiltered against 20 mM Tris/10 mM NaCl, pH 8.0 (10 diavolumes). Either size exclusion chromatography or ultrafiltration/diafiltration can be used to process the pool to a buffer appropriate for formulation.

### Example 3

### Specificity and Kinetics of the Anti-Lewis Y Antibody Calicheamicin Conjugates

To ascertain that conjugation of calicheamicin to the wild-type (hu3S193) and mutant (G193) anti-Lewis Y did not obliterate the binding to Le^{y}, these antibodies and their respective conjugates were subjected to plasmon resonance analysis (BIAcore) and/or FACS analysis. Hu3S193, as well as hu3S193-AcBut-CM, only recognized Le^{y}-BSA and none of the following oligosaccharide antigens: H type I, H type II, sialyl-Le^{a}, sialyl-Le^{x}, sulfo-Le^{a} sulfo-Le^{x}, Le^{a}, Le^{b} or Le^{x}. The kinetics of the binding of hu3S193-AcBut-CM differed from those of hu3S193. The Ka and the Kd of the antibody were also altered by conjugation to CM.

Taken together, the results from BIAcore and FACS analysis indicated that conjugation of CM to hu3S193 or G193 did not affect the specificity for Le^{y}-BSA or for Le^{y} positive cells (data not shown). The altered kinetic parameters of hu3S193-AcBut-CM as compared to hu3S193 did not necessarily translate in different amounts of conjugate or antibody that could bind to N87 cells.

BIAcore Analyis

To confirm the specificity of G193, hu3S193, hu3S193-AcBut-CM and CMD for Le^{y}, the affinity of the antibodies and their CM conjugates for various Le^{y}-related antigens was examined by using surface plasmon resonance analysis using BIAcore 3000. Lewis Y-BSA (30 moles of Lewis Y/mole of BSA) was immobilized on a biosensor chip and exposed to various concentrations of various Lewis Y-reactive agents (2, 4, 8, 12 and 16 nM). G193, hu3S193, hu3S193-AcBut-CM and CMD-193 bind to Le^{y}-BSA with identical affinity and specificity as hu3S193. The kinetic parameters determined by BIAcore rely on the binding of the antibody or conjugate to the artificial Le^{y}-BSA substrate.

The three antibodies had identical kinetic constants. These evaluations indicated that the unconjugated anti-Lewis Y antibodies, G193 and hu3S193, bind Lewis Y-BSA with a modest affinity (KD range 100-300 nM). As shown below in Table 4, conjugation to calicheamicin resulted in a slight reduction in the Lewis Y binding strength of these antibodies in this artificial system. CMD-193 binds Lewis Y antigen with a low affinity and high nanomolar KD.

TABLE 4

| Antibody | K_{D} (M) | K_{A} (1/M) | K_{D} (1/s) | Kₐ (1/Ms) |
|---|---|---|---|---|
| Hu3S193 | 1.3 × 10⁻⁷ | 7.7 × 10⁶ | 4.9 × 10⁻³ | 3.9 × 10⁴ |
| G193 | 1.5 × 10⁻⁷ | 6.6 × 10⁶ | 4.9 × 10⁻³ | 3.3 × 10⁴ |
| CMD-193 | 3.4 × 10⁻⁷ | 2.9 × 10⁶ | 2.5 × 10⁻³ | 7.4 × 10⁴ |

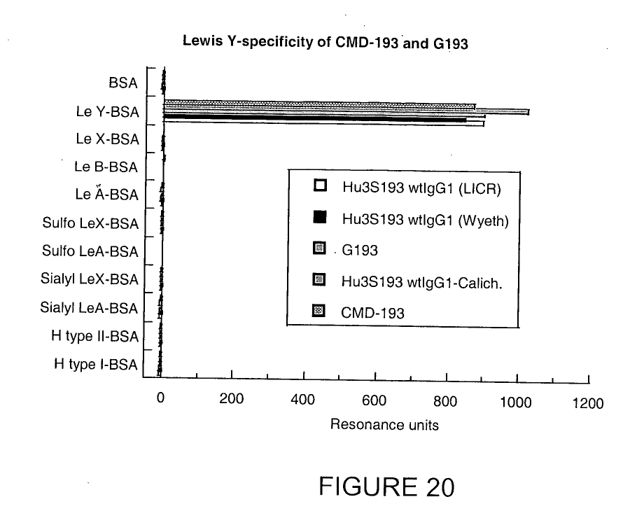

The antibodies and conjugates only recognized Le^{y} and none of the related oligosaccharides. The binding of G193, hu3S193 and their calicheamicin conjugates to various carbohydrate antigens structurally related to Lewis Y was also investigated using biosensor analysis. Various Lewis Y-related antigens conjugated to BSA were immobilized on biosensor chips and exposed to anti-Lewis Y antibodies and their calicheamicin conjugates. These results, shown in FIG. 20, indicated that G193 and CMD-193 are specific for the Lewis Y antigen and do not exhibit any binding even to those antigens that are structurally closer to Lewis Y, the Lewis X and H-2 blood group antigens. These results also further suggest that the conjugation to calicheamicin does not alter the antigen specificity of anti-Lewis Y antibodies.

FACS Analysis

To verify if conjugation affected the binding of hu3S193 to Le^{y+} cells, the amounts of hu3S193 and hu3S193-AcBut-CM that bound to N87 cells were compared by flow cytometry (FACS). The mean channel fluorescence (MCF) obtained after exposing N87 to various concentrations of either hu3S193 or hu3S193-AcBut-CM was similar. N87 cells were incubated with various concentrations of hu3S193 and hu3S193-AcBut-CM. The amount of bound conjugate or antibody was expressed as MCF. Table 5 below shows the flow cytometric detection of the binding of hu3S193 to various carcinoma cell lines (a human IgG1 was used as a control antibody). Lewis Y expression status was arbitrarily assigned based on the ratio of MCF with anti-Lewis Y antibody/MCF with control antibody. A ratio in the range of 3-10 signifies + level, that between 10 and 100 indicates ++ level, that between 100 and 300 indicates +++ level, and that >300 indicates ++++ level of Lewis Y expression. Based on this initial evaluation, Lewis Y-high expressing and low expressing carcinoma cell lines were used in further studies.

TABLE 5

| | | Mean Channel | | |
|---|---|---|---|---|
| | | Fluorescence | | |
| | | | Humanized IgG1 | |
| Carcinoma | Tumor Cell | Control | Anti-Lewis | Expression |
| Type | Line | mAb | Y mAb | Level |
| Breast | MDA-MB-361 | 9 | 298 | ++ |
| | MDA-MB-435 | 3 | 3 | − |
| | MX1 | 25 | 381 | ++ |
| Colon | LOVO | 5 | 62 | ++ |
| | HCT8S11 | 4 | 2221 | ++++ |
| | HCT8S11/R1 | 3 | 1162 | +++ |
| | DLD-1 | 3 | 1167 | +++ |
| | LS174T | 29 | 712 | +++ |
| | HT-29 | 12 | 271 | ++ |
| Epidermoid | A431/LeY | 16 | 912 | +++ |
| | A431 | 19 | 37 | ± |
| | KB | 9 | 106 | ++ |
| Gastric | AGS | 3 | 1063 | ++++ |
| | N87 | 4 | 771 | +++ |
| Lung | L2987 | 4 | 894 | +++ |
| | A549 | 3 | 5 | − |
| | H157 | 2 | 3 | − |
| Prostate | LNCaP | 5 | 50 | + |
| | PC3 | 5 | 41 | + |
| | PC-MM2 | 3 | 19 | + |

G193 and hu3S193 conjugates bind similarly as hu3S193 to Le^{y+} gastric carcinoma cells (N87) in culture. The MCF (mean channel fluorescence)-values determined after exposing N87 monolayers to various concentrations of hu3S193 or G193 were also identical. Hence, in addition to equal binding to Le^{y}-BSA demonstrated with BIAcore, binding of hu3S193, G193, and hu3S193-CM to the naturally displayed Le^{y} was also identical.

Pharmacokinetics

Pharmacokinetic studies with CMD-193 consisted of the following: validation of enzyme-linked immunosorbent assays (ELISAs) to determine concentrations of CMD 193 (rats), the G193 antibody (rats, dogs), unconjugated calicheamicin derivatives (rats, dogs), total calicheamicin derivatives (rats, dogs), and the presence of antibodies specific for CMD-193 in rat serum and for the G193 antibody in dog serum; pharmacokinetic evaluation of the G193 antibody after administration of a single intraperitoneal (IP) dosage of CMD-193 in female nude mice; the in vitro metabolism of NAc gamma calicheamicin dimethyl hydrazide (CM) and NAc gamma calicheamicin DMH AcBut in human liver microsomes and cytosol, and of NAc gamma calicheamicin DMH in HL 60 promyelocytic leukemia cells.

For the in vivo pharmacokinetic study in nude mice, CMD-193 was administered IP in a vehicle that contained 5% sucrose, 0.01% polysorbate 80, 2.92 mg/mL (50 mM) sodium chloride, 2.42 mg/mL (20 mM) Tris, and sterile water for injection, pH adjusted to 8.0. In this study, the loading was approximately 75 mg of calicheamicin derivative/mg of antibody, which is equivalent to approximately 6 moles of calicheamicin/mole of antibody.

The pharmacokinetics of the G193 antibody after single-dose IP administration of CMD 193 at a dosage of 15 mg calicheamicin equivalents/kg (the minimum efficacious dosage (MED)) in female nude mice were characterized by a moderate absorption rate and long apparent terminal half-life (t1/2). The mean area under the concentration-versus-time curve (AUC0-¥) of the G193 antibody was 222 mg·h/mL.

The metabolic fate of NAc gamma calicheamicin DMH and NAc gamma calicheamicin DMH AcBut was examined in vitro in human liver microsomes and cytosol, and the metabolic fate of NAc-gamma calicheamicin DMH was examined in HL-60 promyelocytic leukemia cells. Many metabolites were found after incubation in human liver microsomes and cytosol. The biotransformation pathways in microsomes were hydroxylation and demethylation, whereas the formation of NAc-epsilon calicheamicin and its derivatives appeared to be the major pathways in cytosol. Several metabolites, including NAc-epsilon calicheamicin and its isomer, were produced during incubation with the HL-60 leukemia cells. Common metabolites were observed in both liver and leukemia cell preparations, suggesting that the metabolism of the calicheamicin derivatives may not be cell specific. The detection of NAc-epsilon calicheamicin and its derivatives in cells supports the hypothesis that the reactive diradical species of NAc epsilon calicheamicin probably is formed via a glutathione-dependent reduction of the disulfide bond of NAc-gamma calicheamicin DMH within cells.

### Example 4

### Efficacy of Anti-Lewis Y Antibody Calicheamicin Conjugates on In Vitro Growth of Human Carcinoma Cell Lines

The effect of calicheamicin conjugated to both hu3S193 (hu3S193-CM) and G193 (CMD-193) on in vitro growth of human carcinoma cell lines was evaluated against human carcinoma cell lines. The evaluated cell lines included carcinomas that had either high or low expression of the Lewis Y antigen and carcinomas from breast, colon, lung, and prostate. The efficacy of hu3S193-AcBut-CM and CMD-193 were both compared in vitro to that of CM (free drug) and/or various control conjugates.

As is shown in Tables 6 and 7 below, both hu3S193 and CMD-193 were consistently more effective than a control conjugate (e.g., CMA-676) against Lewis Y-expressing carcinoma cells. In contrast, hu3S193 and CMD-193 were either as efficacious or less efficacious than a control conjugate against cells that had low or little expression of the Lewis Y antigen.

HU3S193-CM

Hu3S193-AcBut-CM specifically inhibits growth of Le^{y} expressing carcinoma cells in vitro. Free hu3S193 antibody did not affect the growth of LOVO, L2987, N87 or AGS when used in concentrations ranging from 1×10⁻⁴ to 6.9 ug protein/ml. This range of protein concentration was the equivalent to the amounts of antibody give as a conjugate. The ED₅₀ indicates the dose (ng/ml) at which 50% of the cell culture survives following exposure to CM or to conjugates for 96 h. The ED₅₀ of hu3S193-AcBut-CM was consistently lower in Le^{y} positive cells (re MCF>10) than the ED₅₀ of CMA. The ED₅₀ of hu3S193-AcBut-CM was consistently lower in Le^{y} positive cells (reMCF>10) than the ED₅₀ of CMA.

Interexperimental variation of the ED₅₀ of both conjugates was observed. However, the ED₅₀ range of hu3S193-AcBut-CM was consistently lower than that of CMA when the efficacy of the conjugates on the Le^{y+} AGS cells was tested. In contrast, these ranges were superimposed when the efficacy of both conjugates was determined on the Le^{y-} PC3MM2 cells. This result was unlikely caused by the selection of the two cell lines. A comparison of the ED₅₀s of CMA and hu3S193-AcBut-CM in parallel experiments using Le^{y+} cells showed on average a lower ED₅₀ for hu3S193-AcBut-CM than for CMA (fold CMA<1). This finding was independent of the origin of the cell line, its sensitivity to calicheamicin and its relative amount of Le^{y}. The parameter fold CMA was ≧1 when various Le^{y-} cells were used. Various hu3S193-AcBut-CM conjugate preparations were used for these experiments (22 and 47 μg Calicheamicin per mg protein) indicating that the observations were independent of this variable. Taken together, the results illustrate the selective cytotoxicity of hu3S193-AcBut-CM due to targeting Calicheamicin to Le^{y}.

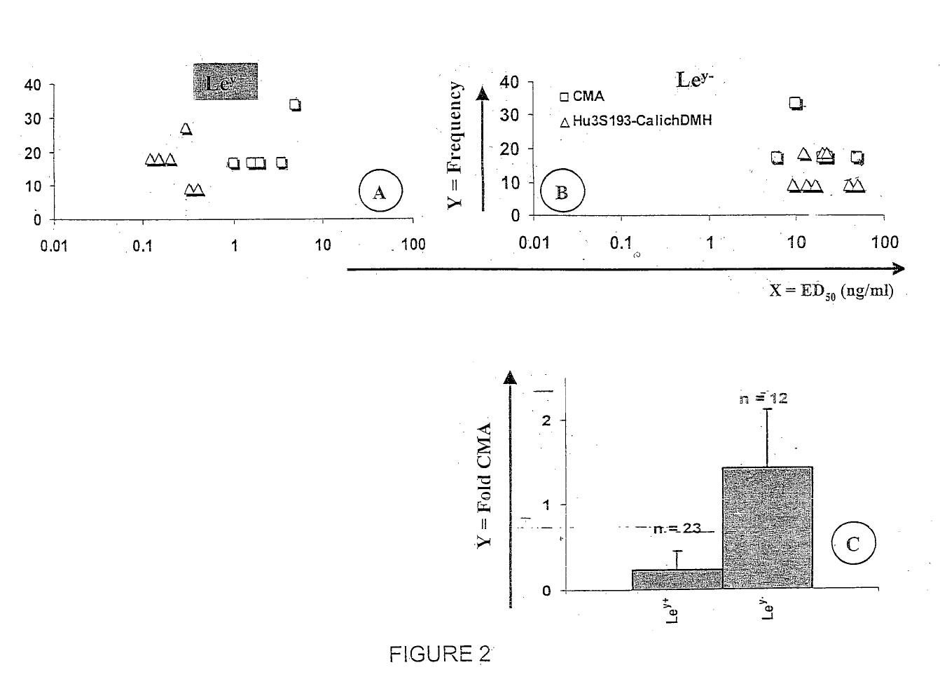

This finding was confirmed by a series of experiments with different batches of hu3S193-AcBut-CM. The conjugate preparations used for these experiments had between 22 and 47 ug CM per mg protein. ED₅₀-values of hu3S193-AcBut-CM and MYLOTARG (CMA) were pooled from 9 experiments and plotted as a function of their frequency of occurrence (see FIGS. 2A and 2B). The efficacy on Le^{y+} cells (AGS, FIG. 2A) was compared to the efficacy on Le^{y-} cells (PC3MM2, FIG. 2B).

For a group of 10 cell lines, the ED₅₀-values of hu3S193-AcBut-CM were also evaluated against those of CMA, used as an internal control in each experiment (fold CMA), where n is the number of independent ED₅₀ determinations (see FIG. 2C).

Despite some interexperimental variation of the ED₅₀, hu3S193-AcBut-CM consistently remained more efficacious (fold-AcBut-CMA<1) than CMA on Le^{y} positive cells. This result illustrates the selective cytotoxicity due to targeting CM to Le^{y}.

TABLE 6

| | | ED₅₀ (nM) Calicheamicin | | | |
|---|---|---|---|---|---|
| | | Equivalents | | | Fold |
| Carcinoma Cell | Lewis Y | | Hu3S193- | CMA- | Selectivity |
| Line | Expression | CM | CM | 676 | Ratio |
| N87 | +++ | 9.0 | 60 | 148 | 2.5 |
| AGS | +++ | 0.01 | 0.24 | 3.50 | 14 |
| HCT8S11 | ++++ | 20 | 16 | >348 | >22 |
| HCTS11R1 | +++ | 21 | 16.5 | >340 | >21 |
| LOVO | ++ | 1.46 | 21 | 45 | 2.1 |
| LNCaP | ++ | 2.1 | <0.007 | 2.80 | >400 |
| NCl-H358 | + | 2.0 | 60 | 90 | 1.5 |
| PC3MM2 | ± | 4.0 | 38 | 16 | 0.42 |

In this experiment, human carcinoma cells were cultured for 96 hr in the presence of increasing concentrations of unconjugated or conjugated calicheamicin (CMA-676 or CMD-193) after which the viable cells in each culture were enumerated using the MTS assay kit. In Table 6 above, CM refers to NAc-Calich DMH, concentrations of both CMA-676 and CMD-193 were expressed in terms of calicheamicin equivalents (nM), and fold selectivity ratio is expressed as the ratio of the ED₅₀ of CMA to the ED₅₀ of CMD. Unconjugated anti-Lewis Y antibodies at 6.7 mg/mL (the highest concentration tested) had no effect on the growth of any of the tumor cell lines examined.

CMD-193

Free G193 antibody did not affect the growth of any investigated cell type when used in concentrations ranging from 5,700 to 6,900 ng protein/ml. As was the case for hu3S193, the ED₅₀ of CMD was consistently lower in Le^{y} positive cells than the ED₅₀ of CMA. The conjugate preparations used for these experiments had between 56 and 88 ug CM per mg protein. Despite some interexperimental variation of the ED₅₀, CMD consistently remained more efficacious (fold-AcBut-CMA<1) than CMA on Le^{y} positive cells. This result illustrated the selective cytotoxicity due to targeting CM to Le^{y}.

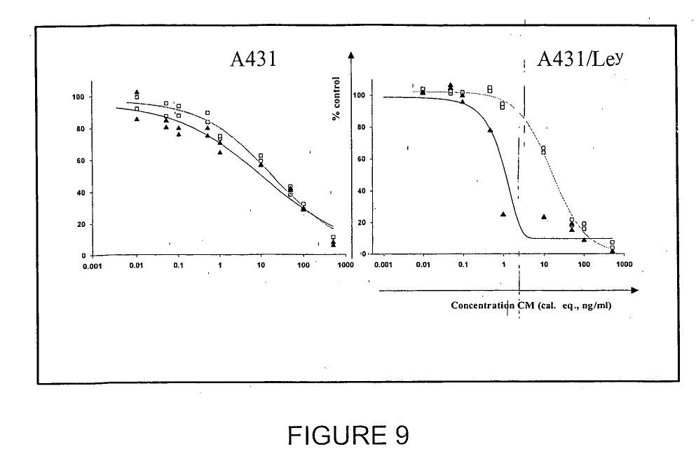

The selectivity of CMD-193 was best illustrated by the comparison of the decay plots of A431 and A431/Le^{y} following treatment with CMA and CMD (FIG. 9).

In this experiment, monolayers of A431 and A431/Le^{y} cells were cultured for 96 h in the presence of CMD or CMA. The number of cells remaining after treatment was determined by a vital dye method and expressed as a percentage of the control. The two types of A431 cells had similar sensitivity to CM. A significant left-shift, relative to CMA, of the CMD decay curve was observed following treatment of the Le^{y} positive cell line (A431/Le^{y}), as shown in FIG. 9B, and not following treatment of the Le^{y} negative cell line (A431), as shown in FIG. 9A.

TABLE 7

| | | ED₅₀ (nM) Calicheamicin | | | Fold |
|---|---|---|---|---|---|
| Carcinoma Cell | Lewis Y | equivalents | | | Selectivity |
| Line | Expression | CM | CMD-193 | CMA-676 | Ratio |
| A431/LeY | +++ | 0.05 | 0.33 | 7.83 | 24 |
| AGS | +++ | 0.03 | 0.1 | 0.68 | 6.9 |
| N87 | +++ | 4.86 | 50.83 | 109.00 | 2.1 |
| L2987 | +++ | 0.30 | 6.00 | 18.60 | 3.0 |
| LS174T | +++ | 0.28 | 15.33 | 22.66 | 1.5 |
| LOVO | ++ | 1.16 | 66.66 | 66.66 | 1.0 |
| LNCaP | ++ | 0.13 | 0.40 | 0.66 | 1.7 |
| A431 | ± | 0.05 | 7.9 | 5.72 | 0.7 |
| PC3MM2 | ± | 2.60 | 19.00 | 6.15 | 0.3 |

In these experiments, human carcinoma cells were cultured for 96 hr in the presence of increasing concentrations of unconjugated or conjugated calicheamicin (CMA-676 or CMD-193), after which the viable cells in each culture were enumerated using the MTS assay kit. In Table 7 above, CM refers to NAc-Calich DMH, concentrations of both CMA-676 and CMD-193 were expressed in terms of calicheamicin equivalents (nM), and fold selectivity ratio is expressed as the ratio of the ED₅₀ of CMA to the ED₅₀ of CMD. Unconjugated anti-Lewis Y antibodies at 6.7 μg/mL (the highest concentration tested) had no effect on the growth of any of the tumor cell lines examined.

### Example 5

### Efficacy of Anti-Lewis Y Antibody Calicheamicin Conjugates on In Vivo Growth of Human Carcinoma Cell xenografts

The antitumor efficacy of calicheamicin conjugated to anti-Lewis Y antibodies was evaluated against human carcinoma xenografts established subcutaneously (SC) in nude mice. The evaluated xenografts included carcinomas that had either high or low expression of the Lewis Y antigen and carcinomas from breast, colon, lung, and prostate. Mice bearing solid tumors with an average mass of 150 to 300 mg were randomized to various treatment groups.

HU3S193-CM

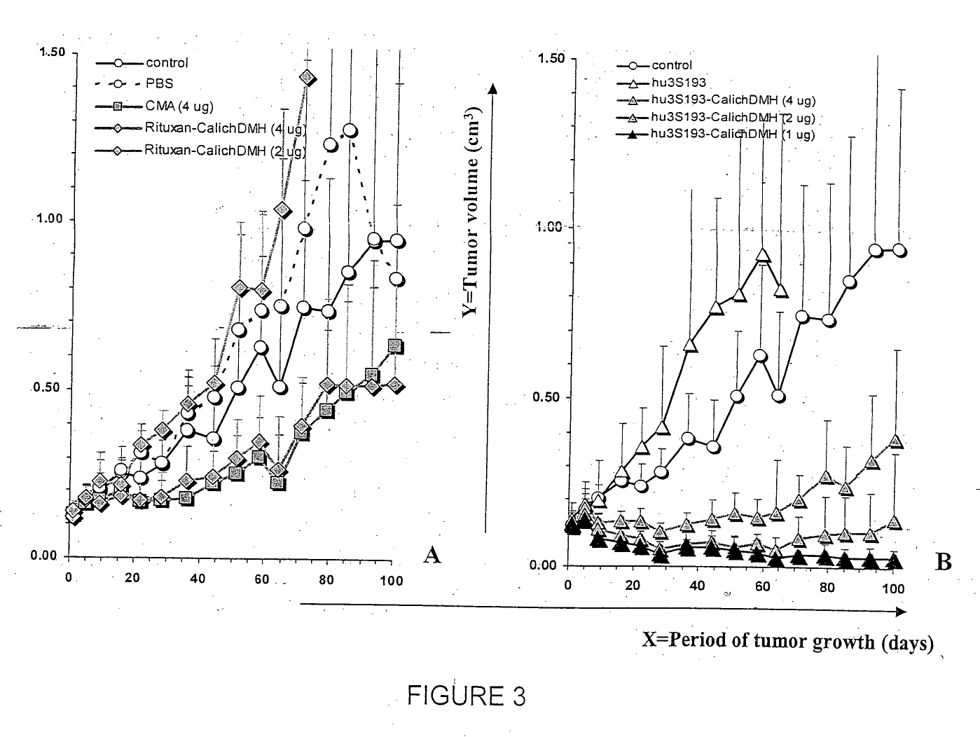

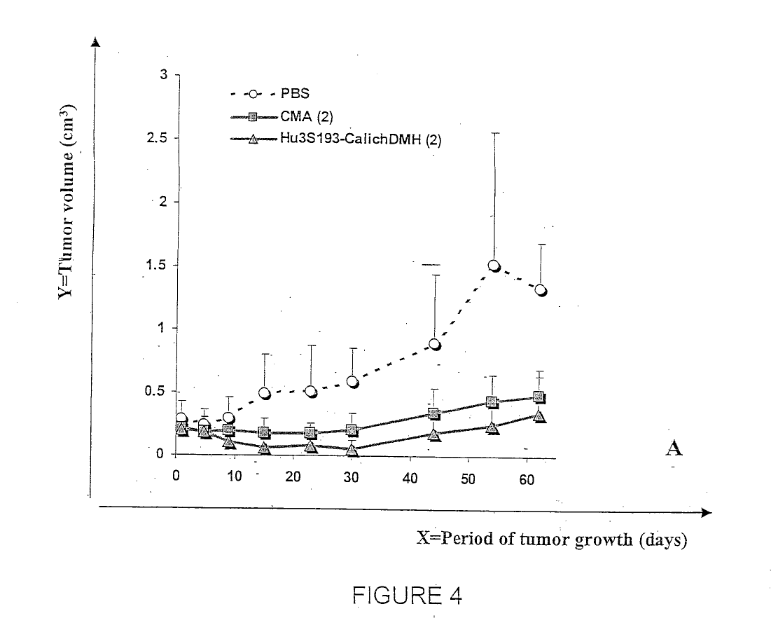

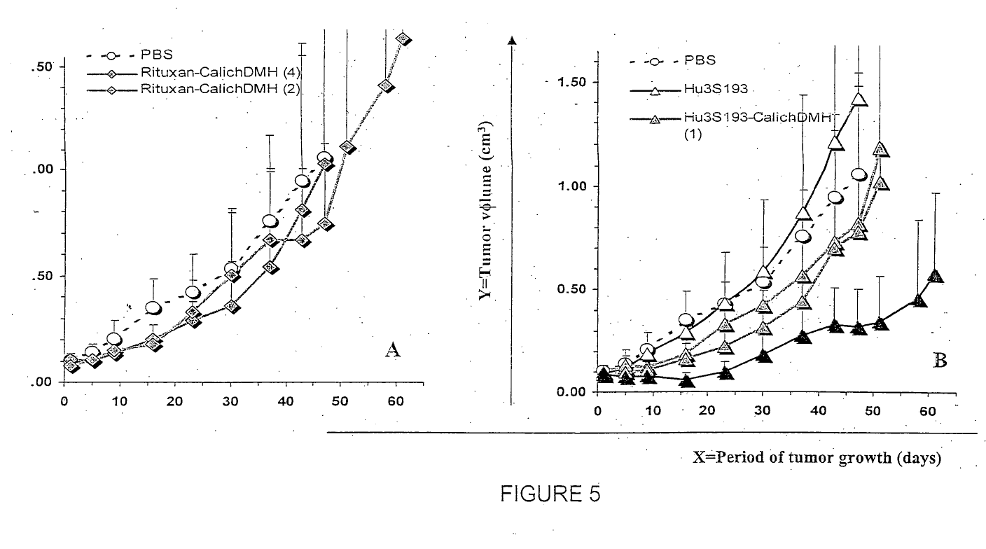

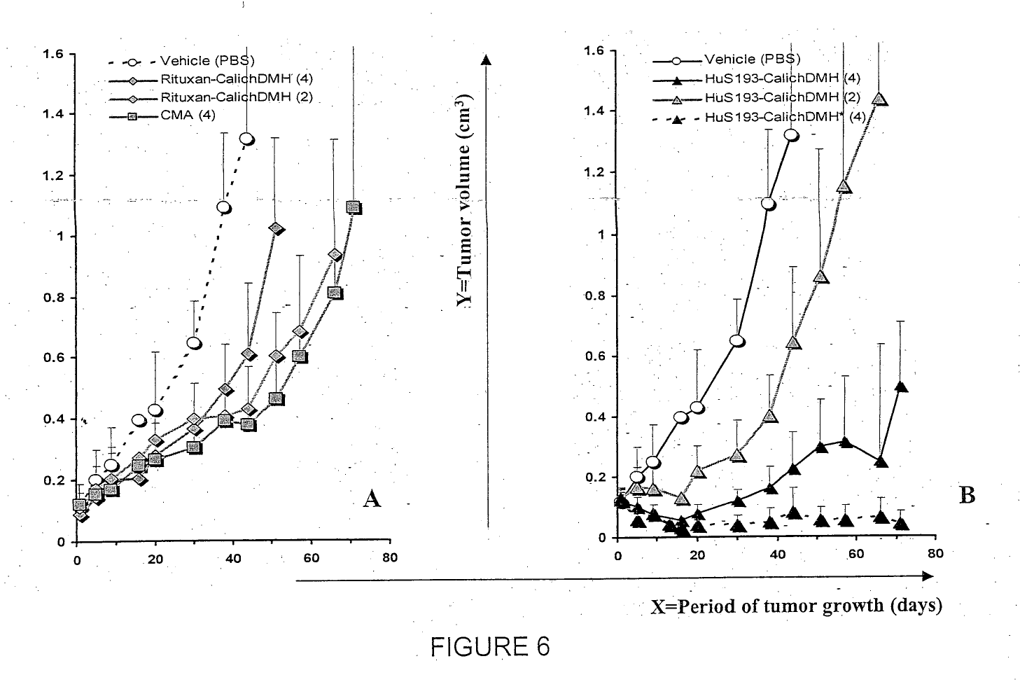

The efficacy in vivo of hu3S193-AcBut-CM was tested on subcutaneous xenografts from gastric (N87, FIG. 3), prostate (LNCaP, FIG. 4) and colon (LOVO, FIGS. 5 and 6) carcinomas. Subcutaneous tumors of N87, LOVO and LNCaP were grown in athymic nude mice (Charles River, Wilmington, Mass.). Female mice of 1.5 to 3 months old were injected with respectively 5×10⁶ N87 or 10⁷ LOVO cells per mouse. LNCaP cells were injected in male nude mice that were 3 months old. To grow tumors, N87 and LNCaP cells had to be mixed (1:1, vol/vol) with MATRIGEL® (Collaborative Biomedical Products, Belford, Mass.) prior to injection. Two perpendicular diameters of the tumor were measured at least once a week by means of calipers. The tumor volume was calculated according to the formula of Attia & Weiss: A²xBx0.4.

Unless indicated otherwise, 3 doses of each conjugate and control were given intraperitoneally with an interval of 4 days (Q4Dx3). In vivo, hu3S193-AcBut-CM inhibited tumor growth in these three separate models. Hu3S193-AcBut-CM cured mice from gastric carcinoma xenografts (N87) having high expression of Lewis Y antigen (FIG. 3). Prostate carcinoma xenografts (LNCaP) ceased to grow following administration of hu3S193-AcBut-CM and inhibition of tumor growth was obtained with colon carcinoma xenografts (LOVO) (FIGS. 5 and 6, respectively). In the LOVO model, the efficacy of hu3S193-AcBut-CM was improved by increasing the amount of the conjugate (FIG. 5).

N87 Gastric Carcinoma Xenografts

Mice bearing N87 (Le^{y+}, CD33⁻ and CD20⁻) xenografts of 100 mm³ were treated with control conjugates (CMA, RITUXAN-AcBut-CM), PBS, hu3S193 or hu3S193-AcBut-CM. Mice in each group received three doses i.p. Conjugates and controls were injected at day 1, 5 and 9. FIG. 3A shows the efficacy of control conjugates and FIG. 3B illustrates the effects of hu3S193 and of its calicheamicin conjugate. The error bars represent the standard deviation of the average tumor volume at each time point. Differences in tumor size among the treated groups of tumor bearing mice have been probed by a 2-tailed Students t-test, the p-values at day 28 are shown in C, and n equals the number of mice per group. At 1, 2, and 4 μg cal.eq/dose/mouse, hu3S193-AcBut-CM significantly inhibited the tumor growth of N87 xenografts (FIG. 3). A cure rate of 100, 60 and 10% was also observed at 4, 2, and 1 μg cal.eq./dose/mouse, respectively, indicating that the size of the xenograft decreases and never exceeds the initial average tumor volume during 100 days following treatment.

LNCaP Prostate Carcinoma Xenografts

LNCaP prostate tumor-bearing mice were treated with hu3S193-AcBut-CM, PBS or the control conjugate CMA. The number between brackets in the legend indicates the amount of calicheamicin per dose per mouse. Differences in tumor size among the treated groups have been probed by a 2-tailed Students t-test. The p-values at day 30 are reported and n equals the number of mice. As shown in FIG. 4, the control conjugates inhibited tumor growth to a lesser extent than hu3S193-AcBut-CM at equivalent or lower doses. Moreover, 0% cure rates were observed following treatment with control conjugates. Hu3S193, when administered at a dose and regimen equivalent to the protein amount (120 μg) given with 4 μg cal. eq. Hu3S193-AcBut-CM, had no effect. Previous experiments showed that administration of Calicheamicin at doses equivalent to hu3S193-AcBut-CM did not inhibit any of the tumor models tested so far. Administration of Calicheamicin has therefore been omitted as a control in the current studies.

LOVO Colon Carcinoma Xenografts

The capacity of hu3S193-AcBut-CM to inhibit tumor growth was also demonstrated in a colon carcinoma (LOVO) model. Mice bearing LOVO xenografts of 100 mm³ were treated with control conjugates (RITUXAN-AcBut-CM, FIG. 5A), PBS (FIGS. 5A and 5B), hu3S193 (FIG. 5B) or hu3S193-AcBut-CM (FIG. 5B). Except for the group treated with Hu3S193-AcBut-CM at 4 μg/dose, mice in each group received three doses i.p. The amount of each dose in calicheamicin equivalents is specified in the legend. Conjugates and controls were injected at day 1, 5 and 9. The groups were designated as follows: hu3S193-AcBut-CM, received an additional regimen of three doses at day 43, 47 and 51. The number of mice per group (n) is reported in C. Differences in tumor size at day 30 were probed for statistical significance by a 2-tailed Students t-test.

Hu3S193 inhibited growth of LOVO-xenografts to a lesser extent than observed with N87-xenografts. Control conjugates (RITUXAN-AcBut-CM or CMA) caused a negligible tumor inhibition. The inhibition caused by hu3S193-AcBut-CM was more prolonged than that of the control conjugates. Thus, differences between, on the one hand, the tumor size following treatment with RITUXAN-AcBut-CM at doses of 4 and 2 μg cal.eq. per mouse (Q4Dx3) and, on the other hand, the tumor size following treatment with PBS were only significant for 16 days (p<0.05).

In contrast, treatment with hu3S193-AcBut-CM at doses of 4, 2 and 1 μg cal.eq. per mouse (Q4Dx3) resulted in statistical differences from the PBS treatment for 43, 22, and 16 days respectively. Mice bearing LOVO xenografts of 100 mm³ were treated with control conjugates: RITUXAN-AcBut-CM and CMA (FIG. 6A), PBS or hu3S193-AcBut-CM (FIG. 6B). Mice in each group received three or four doses i.p. The amount of each dose in calicheamicin equivalents is specified in the legend. Conjugates and controls were injected at day 1, 5 and 9. The group designated: hu3S193-AcBut-CM*, received an additional dose at day 13. The number of mice per group equals n and p-values of a 2-tailed Students t-test were determined.

HCT8S11 Colon Carcinoma XenograftS

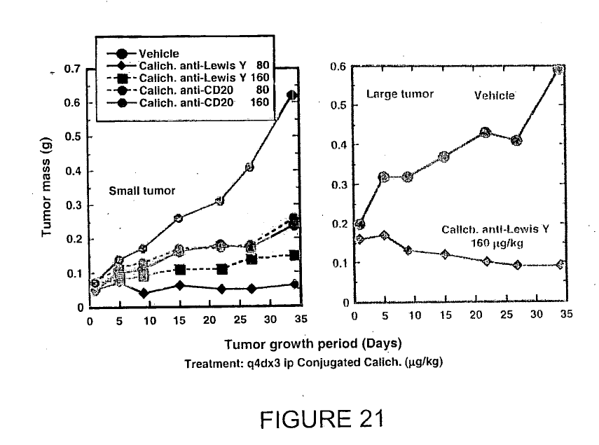

Mice bearing HCT8S11 colon carcinoma xenografts were tested to determine in vivo activity of hu3S193-AcBut-CM. CD20-targeted calicheamicin-conjugated rituximab was used as a nonbinding control. Conjugates were administered IP Q4Dx3 at 80 or 160 mg/kg. FIG. 21 shows that calicheamicin-conjugated hu3S193 was able to cause strong inhibition of growth of HCT8S11 colon carcinoma xenografts, in both small and large tumors. The antitumor activity of Lewis Y-targeted conjugate was always greater than that of nonspecific nonbinding conjugates targeted to either CD20 or CD33.

CMD-193

The efficacy in vivo of CMD-193 was tested on subcutaneous xenografts from gastric (N87), lung (L2987), cervical/epidermoid (A431/Le^{y}) and colon (LS174T and LOVO) carcinomas. Unless indicated otherwise, all conjugates and controls were injected intraperitoneally according to Q4DX3 schedule. To monitor for tumor targeting due to the carrier function of immunoglobulin, CMA was used as a negative control. Based on the studies described below, a dosage of 15 mg/kg of CMD-193 (equivalent to a conjugated antibody protein dosage in the range of 562 to 803 mg/m²) was considered to be the minimum efficacious dose (MED).

N87 Gastric Carcinoma Xenografts

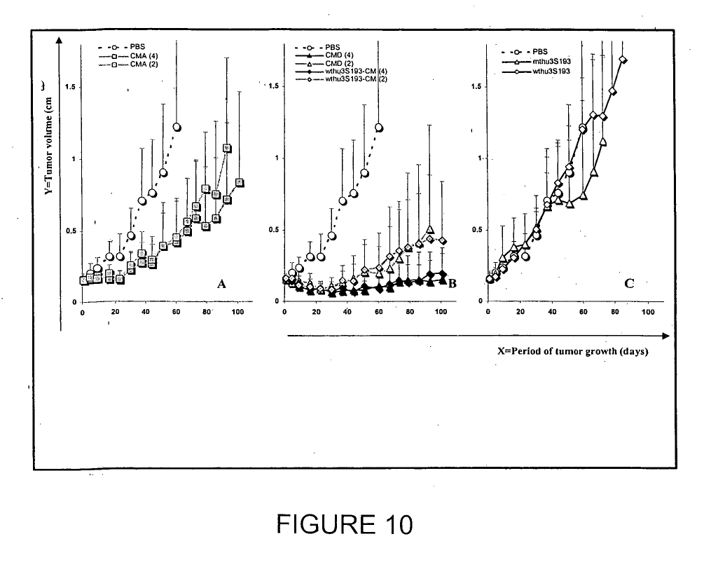

Mice bearing N87 xenografts of 150 mm³ were treated with a control conjugate (CMA), PBS, G193-AcBut-CM, hu3S193-AcBut-CM, G193 or hu3S193. Mice in each group received three doses i.p. The amount of each dose in calicheamicin equivalents is specified in the legends. Conjugates and controls were injected at day 1, 5 and 9. FIG. 10A shows the efficacy of control conjugates. FIG. 10B illustrates the effects of CMD-193 and hu3S193-AcBut-CM, while FIG. 10C. demonstrates the lack of efficacy of free antibody. The error bars represent the standard deviation of the average tumor volume at each time point.

CMA inhibited growth significantly less than either hu3S193-AcBut-CM or CMD at equivalent doses. At 4 μg cal.eq./dose/mouse, hu3S193-AcBut-CM as well as G193-AcBut-CM (CMD) cured mice from N87 xenografts (FIG. 10). Specifically, 40% and 60% of the mice were cured from their tumors after administration of 4 μg cal.eq. of hu3S193-AcBut-CM or CMD, respectively. The term cure indicates that the size of the xenograft decreases and never exceeds the initial average tumor volume during 100 days following treatment. Moreover, the tumor growth inhibition caused by hu3S193-AcBut-CM or CMD was also equivalent at a dose of 2 μg cal.eq./dose/mouse (FIG. 10). Earlier experiments showed that administration of CM in doses equivalent to hu3S193-AcBut-CM never inhibited any of the currently described tumor models (data not shown). Administration of CM has therefore been omitted as a control.

G193 as well as hu3S193 did not Inhibit the Growth of N87 Xenografts.

L2987 Lung Carcinoma Xenografts

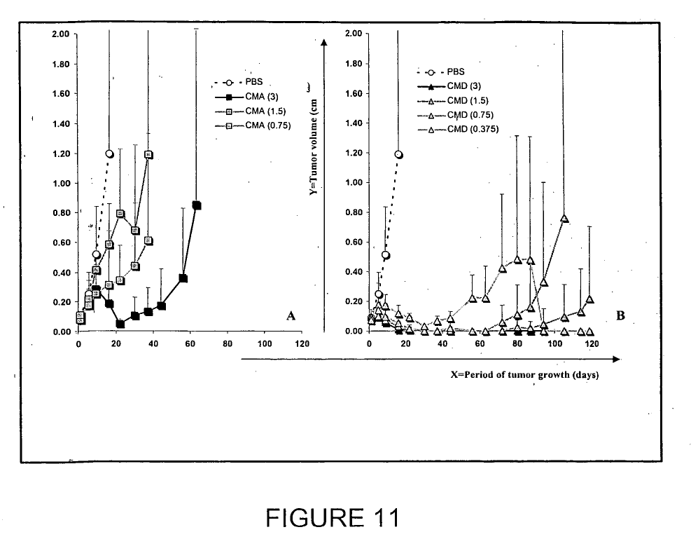

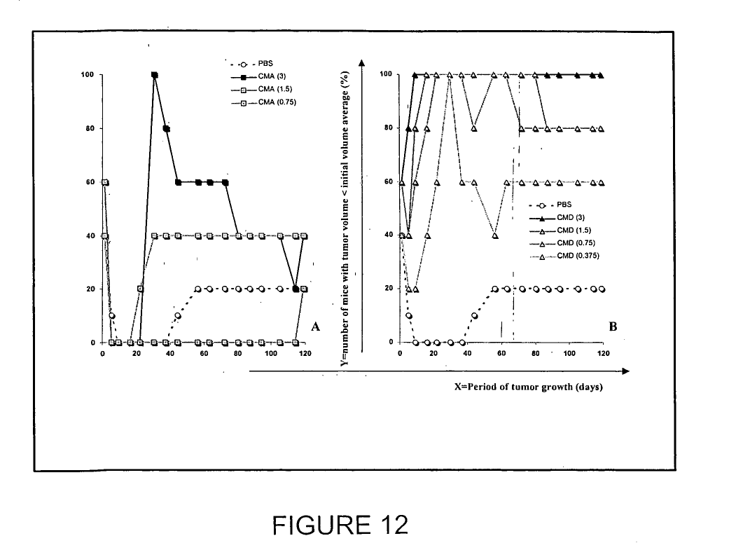

Mice bearing L2987 xenografts of 100 mm³ were treated with a control conjugate (CMA), PBS or CMD. Mice in each group received three doses i.p. The amount of each dose in calicheamicin equivalents is specified in the legends. Conjugates and controls were injected at day 1, 5 and 9. FIG. 11A shows the efficacy of control conjugates and FIG. 11B illustrates the effect of CMD. The error bars represent the standard deviation of the average tumor volume at each time point. The number of mice (expressed as a percentage) with a tumor size smaller than the initial tumor average of each group was plotted as a function of the observation period in FIG. 12. Treatment with CMA (FIG. 12A) or CMD (FIG. 12B) is compared to treatment with vehicle control (PBS).

FIG. 12 shows that CMD inhibited L2987 growth in a dose range from 0.375 to 3 μg/dose/mouse. Interpretation of the selectivity of this inhibition was hampered by two factors. In the first place, CMA exerted a significant growth inhibitory effect in this tumor model (FIG. 12). At lower doses, this inhibition was less than the inhibition caused by CMD. In the second place, spontaneous regression of the tumor occurred in 2 out of 10 mice of the control group (FIG. 12). Notwithstanding, the number of regressed tumors per group was distinctly higher in the groups treated with CMD than in those treated with CMA. CMD also inhibited growth of established L2987 xenografts.

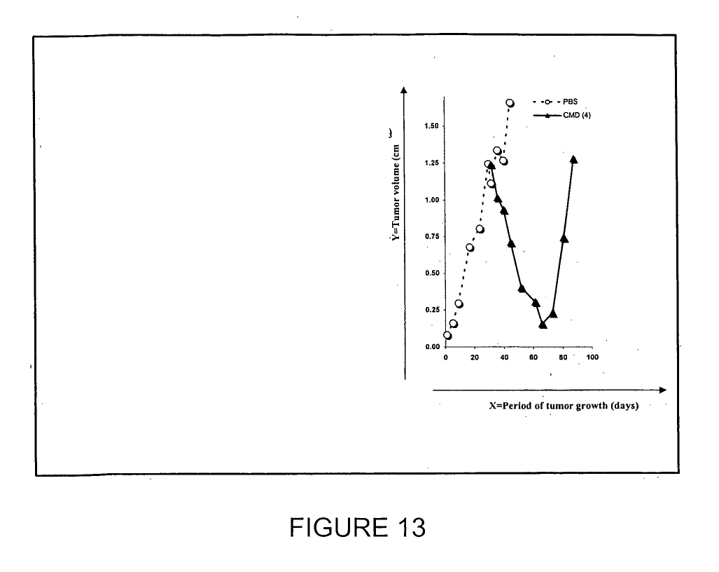

For the experiment shown in FIG. 13, 10 mice received L2987 xenografts. These tumors were grown until they reached an average volume of 1.25 cm³. Three mice with tumor volumes larger than 0.5 cm³ (i.e., 0.66, 1.97 and 1.11 cm³) were treated with 3 doses of 4 μg cal.eq. CMD (Q4DX3). These tumors shrunk after the first dose during a period of 30 days. Sufficient residual disease remained, however, to allow for re-growth of the tumors. One mouse with a tumor of 2.31 cm³ also received 4 ug cal.eq. CMD (Q4DX3). This large tumor did not respond to the therapy and the mouse had to be killed for ethical reasons prior to the third injection. Hence, three doses of 4 ug cal.eq. CMD (Q4DX3) sufficed to inhibit tumor growth of L2987 tumors with volumes between 0.66 and 1.97 cm³ but were inadequate to cure. The error bars represent the standard deviation of the average tumor volume at each time point.

A431/LE^{Y} Epidermoid Carcinoma Xenografts

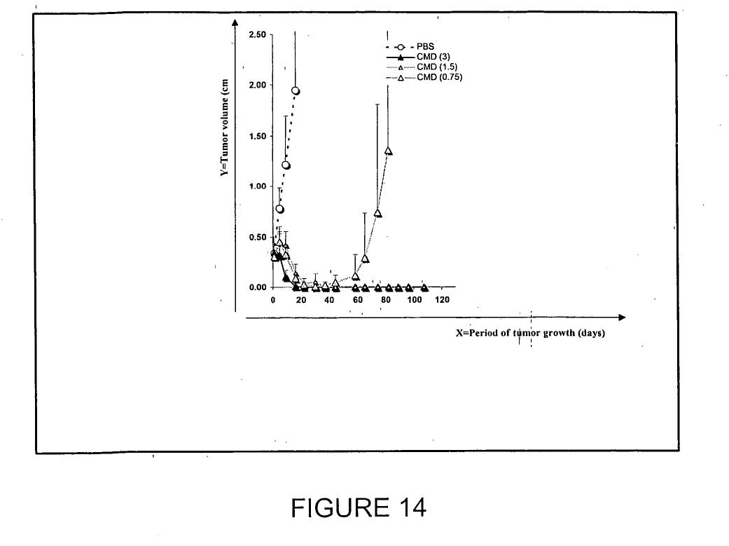

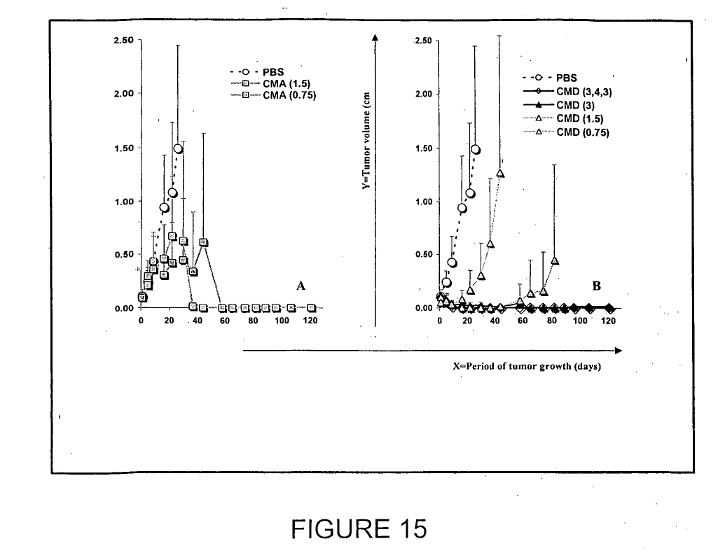

CMD-193 growth inhibition of A431/Le^{y} epidermoid carcinomas was also evaluated. Mice bearing A431/Le^{y} xenografts of approximately 300 mm³ were treated with either PBS or CMD. Mice in each group received three doses i.p. The amount of each dose in calicheamicin equivalents is specified in the legend. Conjugates and controls were injected at day 1, 5, and 9. The error bars represent the standard deviation of the average tumor volume at each time point. Results are shown in FIG. 14. Mice bearing A431/Le^{y} xenografts of approximately 100 mm³ also were treated with control conjugate (CMA), PBS, or CMD. Mice in each group received three doses i.p. The amount of each dose in calicheamicin equivalents is specified in the legend. Conjugates and controls were injected at day 1, 5, and 9. Results are shown in FIG. 15; FIG. 15A shows the efficacy of control conjugates and FIG. 15B illustrates the effects of CMD. The error bars represent the standard deviation of the average tumor volume at each time point

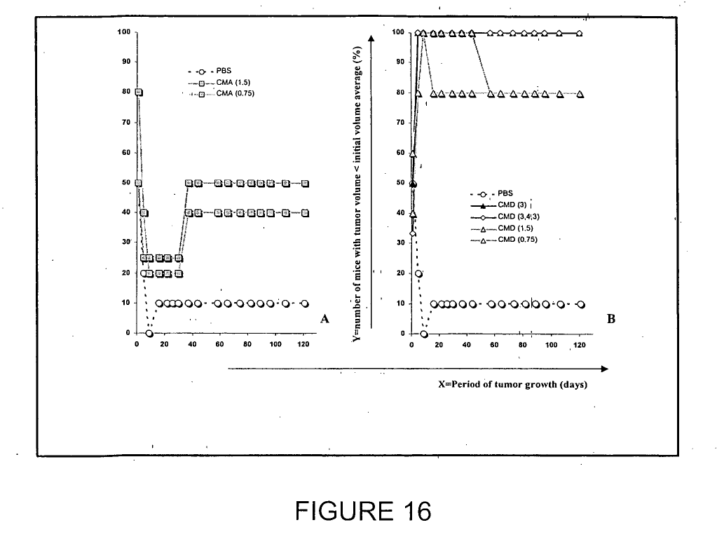

As shown in FIGS. 14 and 15, the interpretation of the specificity of CMD tumor inhibition of A431/Le^{y} was also complicated by spontaneous regressions of the tumors and by growth inhibition caused by CMA. A comparison of the number of cured mice following treatment with equivalent doses of CMD or CMA (FIG. 16) indicated a selective advantage of CMD treatment.

LS174T Colon Carcinoma Xenografts

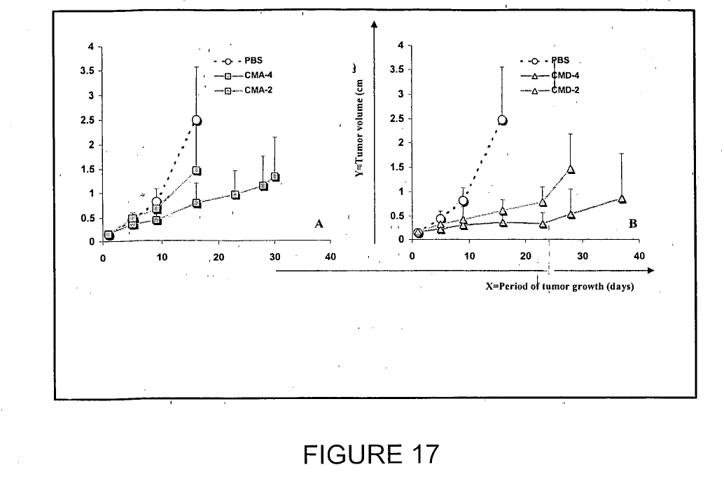

The growth inhibition of LS174T xenografts after treatment with CMD was not as pronounced as with the former tumors; nonetheless, it was more efficacious than the control conjugates (FIG. 17). Mice bearing LS174T xenografts of 150 mm³ were treated with a control conjugate (CMA), PBS or CMD. Mice in each group received three doses i.p. The amount of each dose in calicheamicin equivalents is specified in the legends. Conjugates and controls were injected at day 1, 5 and 9. LS174T tumors proliferated so fast that in the control group all the mice had to be killed within a 51-day period because of the large tumor burden (>2.5 cm³). In the group treated with 4 ug cal.eq. CMD (Q4DX3), 3 out of five mice were killed within 44 days because of a large tumor burden (⅓) or because of necrosis of the tumor (⅔). Of the other two mice one remained tumor-free for 125 days while the other one had developed a small tumor. No cures were observed in the groups treated with either 2 ug cal.eq. CMD (Q4DX3) or 4 ug cal.eq CMA (Q4DX3). In contrast, one out of the five mice treated with 2 μg cal.eq CMA (Q4DX3) was cured. FIG. 17A shows the efficacy of control conjugates and FIG. 17B illustrates the effect of CMD. The error bars represent the standard deviation of the average tumor volume at each time point.

LOVO Colon Carcinoma Xenografts

Mice bearing LOVO xenografts were tested to investigate the potential benefits of regimens different from 4 μg cal.eq./dose/mouse at Q4DX3. Mice bearing LOVO xenografts of approximately 100 mm³ were treated with PBS, G193 or various regimens of a control conjugate CMA or CMD. The amount of each dose in calicheamicin equivalents is specified in the legends. The LOVO-model was chosen for this type of experiment because of the marginal efficacy seen with 4 μg cal.eq. at Q4DX3.

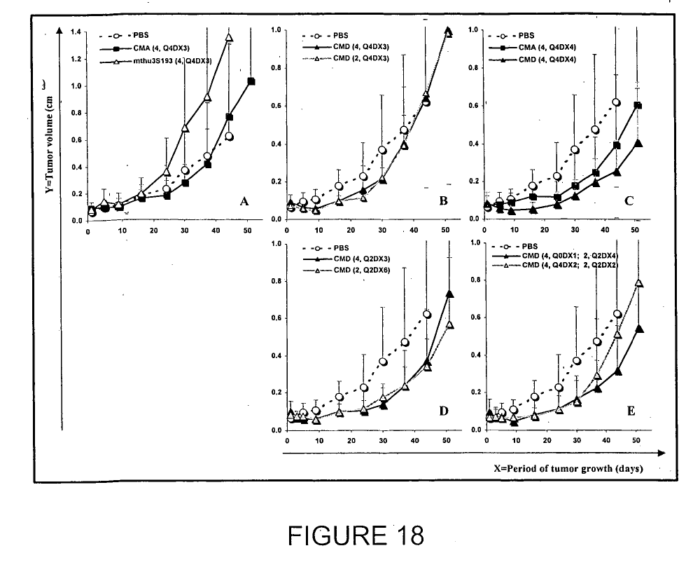

FIG. 18A shows the lack of efficacy of CMA and G193. FIGS. 18B and 18C illustrate the effects of CMD at Q4DX3 and Q4DX4 respectively. FIGS. 18D and 18E show the efficacy of CMD when given with various intervals. The error bars represent the standard deviation of the average tumor volume at each time point. The growth inhibition of LOVO xenografts after treatment with CMD was not as pronounced as with the former tumors. However, it was suggested that addition of a fourth dose, reducing the interval of injection, as well as administering a lower dose more frequently enhanced the efficacy of CMD.

MX1 Breast Carcinoma Xenografts

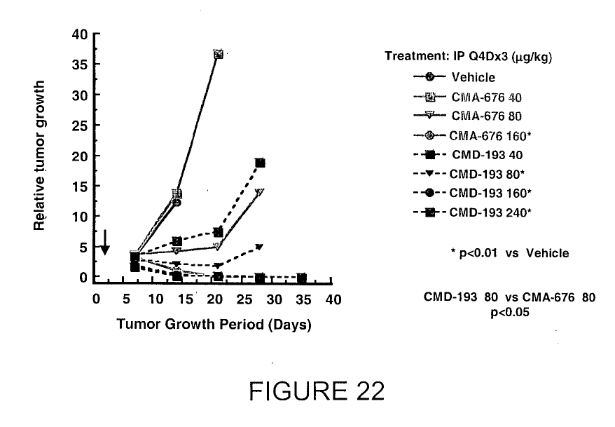

The effect of CMD-193 on the growth of established xenografts of carcinomas that had low expression of the Lewis Y antigen was also examined in MX1 breast carcinoma. CMA-676 was used as a nonbinding control conjugate. Nude mice explanted with MX1 breast carcinoma were treated with various dosages of CMD-193 (40 to 240 mg/kg) or CMA-676 (a negative control). Tumor growth was recorded for at least 35 days. CMD-193 at dosages as low as 80 mg/kg caused significant growth inhibition of MX1 xenograft growth, as shown in FIG. 22. In contrast, CMA-676 was effective only at the highest dosages (160 mg/kg) tested.

Maximum Nonlethal Dose of CMD-193

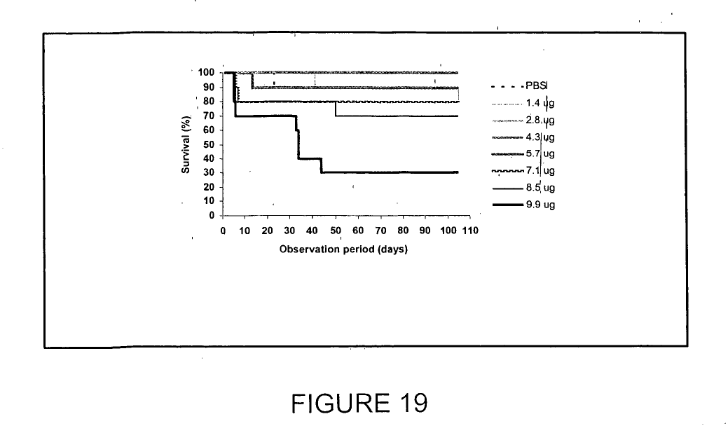

For the experiment illustrated in FIG. 19, eight groups of 10 mice were used. Each group was administered CMD at increasing doses ranging from 0 to 9.9 ug cal.eq. (0 to 396 μg/kg) every 4 days for a total of 3 administrations (Q4DX3) and their survival was monitored for up to 105 days. The control group that was treated with vehicle had one lethality during the entire observation period. The highest dose that led to a similar lethality was 5.7 μg cal.eq. CMD. Because this lethality occurred earlier than in the control group, one could argue that it was due to drug-related toxicity. Dosages of greater than or equal to 284 μg/kg resulted in a significantly higher incidence of lethality in the treated mice and treatment with 7.1, 8.5 and 9.9 ug cal.eq. resulted in not only a distinctly higher incidence of lethality, but also an earlier lethality onset than treatment with the vehicle control. The survival of mice treated with CDM-193 at dosages less than or equal to 228 μg/kg was similar to that in the vehicle-control mice. Taken together these data indicated that the maximum nonlethal dose (MND) of CMD was 5.7 μg cal.eq. (228 μg/kg, which is equivalent to conjugated antibody protein dosages in the range of 8.5 to 12.2 mg/m²) Q4DX3. This MND is considerably higher than the efficacious dose (MED) in most tumor models, for example, given its MED of 15 μg/kg in the L2987 model, CMD-193 exhibits strong antitumor activity with a therapeutic index (MND/MED) of 19.

### Example 6

### Toxicology

The toxicity of the conjugate, CMD 193, was evaluated in single-dose intravenous (IV) toxicity studies in mice and rats, and in dose-ranging and repeat-dose, 4-cycle (cycle is 1 dose/2 weeks) IV toxicity studies in rats and dogs. Toxicokinetic and immunogenicity evaluations were also conducted as part of the 4-cycle toxicity studies in rats and dogs. The genotoxic potential of CMD-193 was evaluated in bacterial reverse mutation and mouse micronucleus assays.

Single- or repeat-dose administration of CMD-193 in rats and dogs (selected for expression of the Lewis Y antigen) produced generally similar in-life effects (decreased body weight and food consumption and hematology changes indicative of bone marrow and lymphoid organ toxicity) and target organ toxicity. Overall, the toxicity of CMD-193 was comparable in rats and dogs. Comparable compound-related findings were observed in males and females. The target organs of toxicity in both species were bone marrow, thymus, and male reproductive organs. The liver (in rats) and the gastrointestinal (GI) tract (in dogs) were also target organs. The observation of CMD-193-related effects in multiple target organs in rats and dogs is consistent with non specific cytotoxicity attributable to the unconjugated calicheamicin derivatives in CMD 193; however, the GI changes in CMD-193-treated dogs may also reflect binding of the G193 antibody to the GI tract epithelium as observed in tissue cross-reactivity studies and subsequent release of the cytotoxic unconjugated calicheamicin derivatives.

Single-Dose IV Studies

In single-dose intravenous (IV) safety pharmacology studies in rats, CMD-193 at dosages of 1.18, 3.54, or 10.69 mg protein/m² did not produce any adverse effects on the central nervous system (CNS) or respiratory systems. In a single-dose cardiovascular safety pharmacology study in dogs, CMD 193 at IV dosages of 1.3 or 6.7 mg protein/m² did not produce adverse changes in heart rate or arterial blood pressure. There was no evidence of morphologic abnormalities, abnormal atrial or ventricular arrhythmias, or compound-related QTc prolongation in any of the electrocardiograms (ECGs) examined at 6.7 mg protein/m² (ECGs were not examined at 1.3 mg protein/m²).

When administered IV as a single dose, the highest non-lethal dosages of CMD-193 were 15.30 mg protein/m² in mice and 30.09 mg protein/m² in rats; these were the maximum feasible dosages based on the maximum concentration of 76 mg/mL (calicheamicin equivalents) and the maximum dose volume of 5 mukg. The dosages that did not produce adverse effects were 15.30 mg protein/m² for mice and 15.81 mg protein/m² for rats.

Dose-Ranging Studies

In dose-ranging studies with CMD-193, moribundity necessitating euthanasia occurred in 1 dog at the highest tested single dosage of 12 mg protein/m²; moribundity was attributed to CMD 193 related gastroenteric changes of slight-to-moderate mucosal degeneration and necrosis. No rats were found dead or electively euthanized at any dosage tested (up to 30.09 mg protein/m²).

4-Cycle Studies

In the 4-cycle toxicity studies, the dosages of CMD-193 administered were 0.55, 1.98, or 5.55 mg protein/m²/cycle in rats and 0.36, 1.2, or 3.59 mg protein/m²/cycle in dogs. In these studies, G193 antibody alone was administered at 5.55 mg protein/m²/cycle in rats and 3.59 mg protein/m²/cycle in dogs. The maximum tolerated dosages (MTDs) of CMD-193 were 5.55 mg protein/m²/cycle in rats and 3.59 mg protein/m²/cycle in dogs (the highest dosages administered); these dosages did not elicit dose-limiting or life-threatening toxicity. In the 4-cycle study in rats, a no-observed-adverse-effect level (NOAEL) for CMD-193 was not established in males based on the microscopic findings (testicular tubular atrophy) observed at 0.55 mg protein/m²/cycle. Based on hepatocellular karyomegaly/cytomegaly in both males and females at 5.55 mg protein/m²/cycle, the NOAEL in females in the 4-cycle study in rats was 1.98 mg protein/m²/cycle. In the 4-cycle dog study, the NOAEL for CMD-193 in males was not established based on microscopic findings (testicular tubular degeneration with secondary epididymal hypospermia and slight epididymal epithelial degeneration) at 0.36 mg protein/m²/cycle. Based on microscopic findings of mucosal epithelial degeneration in the GI tract at 3.59 mg protein/m²/cycle in both males and females, the NOAEL for CMD-193 in females in the 4-cycle study in dogs was 1.2 mg protein/m²/cycle.

In the 4-cycle toxicity study in rats, the tested dosage of the G193 antibody alone of 5.55 mg protein/m²/cycle did not result in any G193 antibody-related toxicity. In the 4 cycle toxicity study in dogs, the tested dosage of the G193 antibody alone of 3.59 mg protein/m²/cycle did not result in dose limiting or life threatening toxicity. G193 antibody-related toxicity in this study in dogs included slight testicular tubular degeneration and slight gastric mucosal degeneration.

Toxicokinetic evaluations of the G193 antibody, unconjugated (free) calicheamicin derivatives, and total calicheamicin derivatives (rats only), as well as determination of the presence of antibodies specific for CMD-193 in rat serum and for the G193 antibody in dog serum, were conducted as part of the 4 cycle repeat-dose IV toxicity studies.

Genotoxic Studies

CMD-193 was negative for mutagenicity in the bacterial reverse mutation assay but clastogenic in an in vivo mouse micronucleus assay. The positive response in this assay was expected and is consistent with the induction of DNA breaks (clastogenicity) by the calicheamicins and other enediyne antitumor antibiotics.

Cross-Reactivity Studies

In cross reactivity studies, unconjugated G193 antibody showed specific staining in the salivary gland and GI tract of rats and dogs, and in the pancreas and liver (biliary epithelium) in dogs only. In an additional study, the most prominent and consistent staining with the G193 antibody was in the GI tract (epithelium of the large intestine and stomach), urinary bladder (epithelium), vagina (epithelium), and pituitary (endocrine cells of the adenohypophysis) in rats, dogs, and humans. Since the G193 antibody component of CMD-193 cross-reacted with tissues associated with expression of the Lewis Y antigen in rats, dogs, and humans, these results demonstrate that rats and dogs are appropriate species for the nonclinical studies that were conducted with CMD 193.

### Example 7

### Stable Formulations of Anti-Lewis Y Antibody Calicheamicin Conjugates

Stable formulations of anti-Lewis Y antibody calicheamicin conjugates (hu3S193-AcBut-CM and CMD-193) for in vivo administration were prepared. Approximately 19 mg of CMD-193 in 20 mM TRIS (pH 8.0), and 100 mM sodium chloride was formulated as follows according to Tables 8 or 9.

TABLE 8

| Ingredient | Content |
|---|---|
| CMD-193 | 1 mg/mL |
| Sucrose | 5% |
| TRIS | 20 mM |
| Sodium Chloride | 50 mM |
| Hydrochloric Acid, 1 N | pH adjusted to 7.5 and 8.0 |
| Water for Injection | q.s |

TABLE 9

| Active Ingredients | Inactive Ingredients |
|---|---|
| CMD-193 | 5% Sucrose |
| (5 mg) | 0.01% Tween 80 |
| Fill volume 5 mL | 10 mM Sodium chloride |
| | 20 mM TRIS |
| | pH adjusted to 8.0 with HCl |

Two batches of CMD-193 were lyophilized. The difference in the formulations was of the pH, as one was buffered at pH 7.5, and the other at pH 8.0. Four vials of the pH 8.0 formulation were reconstituted with water for injection and combined in a polypropylene tube. The pH was measured and then the solution was divided into four portions and put at 25° C. Similarly four vials were used to set up a similar study at pH 7.5. When the solutions were combined, the pH had to be readjusted with 0.1 N Hydrochloric acid to 7.5. Four more vials were also used to make a solution at pH 7.0. The solutions were given for initial analysis and the rest was kept in the stability chambers at 25° C. The solutions were then analyzed after 2 days and 7 days and results are shown below in Table 10 (stability of CMD-193 bulk solution at pH 7.0, 7.5, and 8.0 for 1 week at 25° C.). The solutions were observed to be cloudy and clear precipitate were observed in pH 7.0 and pH 7.5 solutions after 1 week at 25° C. Based upon the results, the solution buffered at pH 8.0 resulted in the best stability of the three cases and TRIS was selected as the buffer.

TABLE 10

| | | | | Unconjugated | | | | | |
|---|---|---|---|---|---|---|---|---|---|
| | | | | Calicheamicin | | | Aggregates | | |
| Days | pH | | | (μg/mg of protein) | | | (%) | | |
| pH | 7.0 | 7.5 | 8.0 | 7.0 | 7.5 | 8.0 | 7.0 | 7.5 | 8.0 |
| 0 | 7.056 | 7.432 | 8.039 | 0.50 | 0.41 | 0.61 | 3.25 | 3.38 | 3.33 |
| 2 | | | | 2.09 | 1.83 | 1.69 | 2.47 | 3.06 | 3.25 |
| 7 | 6.919 | 7.321 | 7.933 | 6.24 | 5.52 | 4.63 | 1.87 | 2.60 | 3.34 |

In another example, approximately 35 mg of CMD-193 in 20 mM TRIS (pH 8.0) and 100 mM sodium chloride were used. From this, two additional formulations of CMD-193 were manufactured. The first formulation contained 5% sucrose and was buffered with TRIS at pH 8.0. The final formulation is described below in Table 11.

TABLE 11

| Ingredient | Content |
|---|---|
| CMD-193 | 1 mg/mL |
| Sucrose | 5% |
| TRIS | 20 mM |
| Sodium Chloride | 50 mM |
| Hydrochloric Acid, 1 N | pH adjusted to 8.0 |
| Water for Injection | q.s |

To manufacture the second formulation, the concentrate of CMD-193 used (total protein 2.23 mg/mL) was diluted with water for injection such that the concentration of the protein was 1 mg/mL. This solution was then centrifuged using centricon filter units that are permeable to molecules less than 30,000 Daltons.

When the solution volume was halved, it was then diluted with a 5 mM K₂HPO₄, 50 mM NaCl, 10% sucrose solution. The final formulation is described below in Table 12.

TABLE 12

| Ingredient | Content |
|---|---|
| CMD-193 | 1 mg/mL |
| Sucrose | 5% |
| K₂HPO₄ (Buffer Exchanged) | 5 mM |
| Sodium Chloride | 50 mM |
| Hydrochloric Acid, 1 N | pH adjusted to 7.5 |
| Water for Injection | q.s |

The vials of CMD-193 manufactured at pH 7.5 and pH 8.0 were reconstituted with various solutions listed in Table 13 (second formulation), which shows a visual inspection of CMD-193 reconstituted with different solutions, and the vials were observed for visual particles. In all cases, the solutions buffered at pH 8.0 were clearer than those at pH 7.5. Addition of surfactant was beneficial in all cases. The precipitate in the vials reconstituted with water for injection (wfi) were filtered and collected for microscopic examination.

TABLE 13

| pH | Reconstituting Solution | Observation |
|---|---|---|
| 7.5 | wfi | Precipitate |
| 7.5 | 0.01% Tween 80 | Clear |
| 7.5 | 0.1% Tween 80 | Clear |
| 7.5 | 0.1% Poloxamer 188 | Slight turbidity |
| 7.5 | 10% Propylene Glycol | Precipitate |
| 8.0 | wfi | Precipitate |
| 8.0 | 0.01% Tween 80 | Clear |
| 8.0 | 0.1% Tween 80 | Clear |
| 8.0 | 0.1% Poloxamer 188 | Clear |
| 8.0 | 10% Propylene Glycol | Clear |

Based upon the above, addition of a surfactant to the solution was found to be necessary to ensure solubility. A choice of Tween 80 (0.01%) was used to maintain solubility of 1 mg/ml. The final formulation contained 5% sucrose, 0.01% Tween 80, 20 mM TRIS (pH 8.0), and 50 mM Sodium Chloride)

Two more batches of CMD-193 were used. The first batch was purified using HIC followed by ultra-filtration, and the second batch was directly passed through ultra-filtration process after conjugation. The two formulations were formulated as below in Tables 13 and 14.

TABLE 14

| Ingredient | Content |
|---|---|
| CMD-193 | 1 mg/mL |
| Sucrose | 5% |
| TRIS | 20 mM |
| Sodium Chloride | 50 mM |
| Hydrochloric Acid, 1 N | pH adjusted to 8.0 |
| Water for Injection | q.s |

Stability of the bulk solution at 5° C. (Table 15) and the lyophilized product at 25° C. (Table 16) was performed and is summarized below.

TABLE 15

| | Initial | 1 week | 2 weeks |
|---|---|---|---|
| LIMS # | 200272733 | 200273196 | 200273639 |
| App & Desc; cake | conforms | conforms | conforms |
| App & Desc; | conforms | conforms | conforms |
| Reconstituted | | | |
| Protein Content | 1.05 | 1.06 | 1.06 |
| (mg/mL) | | | |
| Total Calicheamicin | 66 | | |
| (μg/mg of protein) | | | |
| Unconjugated | 1.13 | 2.31 | 2.64 |
| Calicheamicin | | | |
| (% total calicheamicin) | | | |
| Aggregates (%) | 3.02 | 3.39 | 3.32 |
| pH Reconstituted | 7.83 | 7.79 | 7.71 |
| SDS-PAGE Reduced | 100 | | 100 |
| (%) | | | |
| Antigen Binding | 108 | | |
| ELISA | | | |
| Unconjugated | 1.77 | | |
| Antibody (%) | | | |

TABLE 16

| | Initial | 2 weeks | 4 weeks |
|---|---|---|---|
| LIMS # | 200273198 | 200274024 | 200274713 |
| | 200273204 | | 200274715 |
| App & Desc; cake | conforms | conforms | conforms |
| App & Desc; | conforms | conforms | conforms |
| Reconstituted | | | |
| Protein Content | 0.99 | 0.99 | 0.98 |
| (mg/vial) | | | |
| Total Calicheamicin | 67 | | 67 |
| (μg/mg of protein) | | | |
| Unconjugated | 1.11 | 1.59 | 1.56 |
| Calicheamicin | | | |
| (% of total | | | |
| calicheamicin) | | | |
| Aggregates (%) | 3.32 | 3.17 | 3.18 |
| pH Reconstituted | 7.76 | 7.78 | 7.75 |
| Moisture | 0.95 | | 1.19 |
| SDS-PAGE Reduced | | | 100 |
| (%) | | | |
| Antigen Binding | | | 99 |
| ELISA (%) | | | |

Dilution and Administration

CMD-193 for injection is supplied as a sterile white, preservative-free, freeze-dried powder in a 20-mL amber glass vial. Each single-vial package contains 5 mg of CMD 193 freeze-dried powder. CMD-193 for injection can be refrigerated (2 to 8° C./36 to 46° F.) and protected from light.

The drug product is light sensitive and can be protected from direct and indirect sunlight and unshielded fluorescent light during both preparation and administration. All preparation is preferably done inside a biologic safety hood. The lyophilized drug may be reconstituted without equilibration of the vial to room temperature. Sterile syringes are used to reconstitute the contents of each vial with 5 mL of sterile water for injection, USP. Gentle swirling can be used to aid this process. After reconstitution and before administration, each vial of drug is inspected visually for particulate matter and discoloration. The final concentration of the reconstituted solution is 1 mg/mL.

Sterile water for injection, USP containing benzyl alcohol or any other preservative is not recommended for reconstitution of CMD-193 for Injection.

Once reconstituted, the drug solution is further diluted into 0.9% Sodium Chloride injection, USP and administered within 4 hours after reconstitution of the vials. Reconstituted vials of CMD-193 for Injection should never be allowed to freeze.

To produce the final dose for the administration, the appropriate amount of reconstituted drug is injected into sufficient 0.9% Sodium Chloride Injection, USP to produce a final volume of 50 mL. The admixture bag or container is composed of polyolefin or contain a polyethylene-lined contact surface and with an ultraviolet UV light protector.

CMD-193 for Injection should not be administered as an IV push or bolus.

The patient receives the admixture solution (total dose) by IV infusion at a constant rate over a 1-hour (±15 minutes) period via a programmable infusion pump. Although the infusion container should be protected from light, it is not necessary to protect the infusion tubing from light. Infusion tubing may be either polyolefin or polyethylene-lined. In-line filters should not be used with CMD-193 administration.

All references and patents cited above are incorporated herein by reference. Numerous modifications and variations of the present inventions are included in the above-identified specification and are expected to be obvious to one of skill in the art. Such modifications and alterations to the conjugation process, the conjugates made by the process, and to the compositions/formulations comprising conjugates are believed to be encompassed within the scope of the claims.

What is claimed is:

1. A process for preparing a calicheamicin conjugate comprising reacting at a pH of about 7 to about 9 (i) an activated calicheamicin-hydrolyzable linker derivative and (ii) an IgG1 antibody in the presence of a member of the deoxycholate family or a salt thereof.

2. The process of claim 1, wherein the deoxycholate family member has one of the following structures: 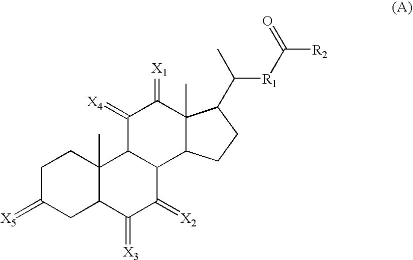
wherein
two of X₁ through X₅ are H or OH and the other three are independently either O or H;
R₁ is (CH₂)ₙ where n is 0-4 and
R₂ is OH, NH(CH₂)ₘCOOH, NH(CH₂)ₘSO₃H, or NH(CH₂)ₘPO₃H₂ where m is 1-4.
OR 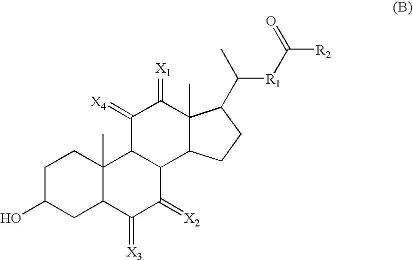
wherein
one of X₁ through X₄ is H or OH and the other three are independently either O or H;
R₁ is (CH₂)ₙ where n is 0-2 and
R₂ is OH, NH(CH₂)ₘCOOH, or NH(CH₂)ₘSO₃H, where and m is 2.
OR 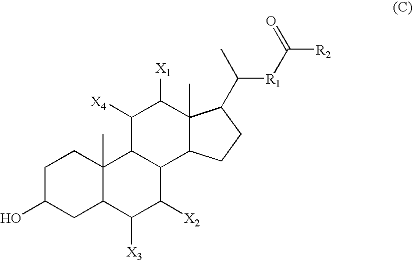
wherein
one of X₁ through X₄ is OH and the other three are H;
R₁ is (CH₂)ₙ where n is 0-2 and
R₂ is OH, NH(CH₂)₂SO₃H.

3. The process of claim 1, wherein the deoxycholate family member is chenodeoxycholic acid, hyodeoxycholate, urosodeoxycholic acid, glycodeoxycholic acid, taurodeoxycholic acid, tauroursodeoxycholic, or taurochenodeoxycholic.

4. The process of claim 1, wherein the deoxycholate family member is deoxycholic acid at a concentration of about 10 mM.

5. The process of claim 1, wherein the calicheamicin derivative is about 3 to about 9% by weight of the IgG1 antibody.

6. The process of claim 5, wherein the calicheamicin derivative is about 7% by weight of the IgG1 antibody.

7. The process of claim 1, wherein the IgG1 antibody is an anti-Lewis Y antibody.

8. The process of claim 7, wherein the anti-Lewis Y antibody is G193 or Hu3S193.

9. The process of claim 1, wherein the calicheamicin derivative is an N-acyl derivative of calicheamicin or a disulfide analog of calicheamicin.

10. The process of claim 9, wherein the calicheamicin derivative is N-acetyl gamma calicheamicin dimethyl hydrazide (N-acetyl calicheamicin DMH).

11. The process of claim 1, wherein the hydrolyzable linker is 4-(4-acetylephenoxy) butanoic acid (AcBut).

12. The process of claim 1, wherein the pH is about 8.2.

13. The process of claim 1, wherein the process further comprises purifying the calicheamicin conjugate.

14. The process of claim 13, wherein purification comprises chromatographic separation and ultrafiltration/diafiltration.

15. The process of claim 14, wherein the chromatographic separation is size exclusion chromatography (SEC) or hydrophobic interaction chromatography (HIC).

16. The process of claim 13, wherein following the purification step, the average loading of the conjugate is from about 5 to about 7 moles of calicheamicin per mole of IgG1 antibody.

17. The process of claim 13, wherein following the purification step, the low conjugated fraction (LCF) of the conjugate is less than about 10%.

18. A calicheamicin conjugate prepared by the process of claim 1.

19. A calicheamicin conjugate prepared by the process of claim 13.

20. A calicheamicin conjugate prepared by the process of claim 13, wherein the calicheamicin derivative is N-acetyl gamma calicheamicin dimethyl hydrazide (N-acetyl calicheamicin DMH), the hydrolyzable linker is 4-(4-acetylephenoxy) butanoic acid (AcBut), the deoxycholate family member is deoxycholic acid at a concentration of about 10 mM, the pH is about 8.2, and wherein following the purification step, the average loading of the calicheamicin conjugate is from about 5 to about 7 moles of calicheamicin per mole of IgG1 antibody and the low conjugated fraction (LCF) of the conjugate is less than about 10%.

21. A composition comprising a conjugate of a calicheamicin-hydrolyzable linker derivative covalently attached to an anti-Lewis Y antibody.

22. The composition of claim 21, wherein the calicheamicin derivative is an N-acyl derivative of calicheamicin or a disulfide analog of calicheamicin.

23. The composition of claim 22, wherein the calicheamicin derivative is N-acetyl gamma calicheamicin dimethyl hydrazide (N-acetyl calicheamicin DMH).

24. The composition of claim 21, wherein the hydrolyzable linker is 4-(4-acetylephenoxy) butanoic acid (AcBut).

25. The composition of claim 21, wherein the anti-Lewis Y antibody is G193 or Hu3S193.

26. The composition of claim 21, wherein the average loading is from about 5 to about 7 moles of calicheamicin per mole of anti-Lewis Y antibody.

27. The composition of claim 21, wherein the conjugate has a K_{D} of about 1×10⁻⁷ M to about 4×10⁻⁷ M.

28. The composition of claim 21, wherein the conjugate has a K_{D} of about 3.4×10⁻⁷ M.

29. The composition of claim 21, wherein the conjugate binds the Lewis Y antigen and does not bind Lewis X and H-2 blood group antigens.

30. The composition of claim 21, wherein the conjugate has cytotoxic activity.

31. The composition of claim 21, wherein the conjugate has anti-tumor activity.

32. The composition of claim 21, wherein the conjugate is present in a therapeutically effective amount.

33. A composition comprising a conjugate of N-acetyl gamma calicheamicin dimethyl hydrazide-4-(4-acetylephenoxy) butanoic acid (N-acetyl calicheamicin DMH-AcBut) covalently linked to G193, wherein the average loading is from about 5 to about 7 moles of N-acetyl calicheamicin DMH per mole of G193 and the low conjugated fraction (LCF) of the conjugate is less than about 10%.

34. A process for preserving biological activity of the composition of claim 21 comprising contacting the composition with a cryoprotectant, a surfactant, a buffering agent, and an electrolyte in a solution; and lyophilizing the solution.

35. The process of claim 34, wherein the conjugate is at a concentration of about 0.5 mg/mL to about 2 mg/mL.

36. The process of claim 35, wherein the conjugate is at a concentration of 1 mg/mL.

37. The process of claim 34, wherein the cryoprotectant is at a concentration of about 1.5% to about 6% by weight.

38. The process of claim 34, wherein the cryoprotectant is a sugar alcohol or a carbohydrate.

39. The process of claim 38, wherein the cryoprotectant is trehalose, mannitol, or sorbitol.

40. The process of claim 38, wherein the cryoprotectant is sucrose at a concentration of about 5%.

41. The process of claim 34, wherein the surfactant is at a concentration of about 0.005% to about 0.05%.

42. The process of claim 41, wherein the surfactant is Polysorbate 80 at a concentration of 0.01% by weight or Tween 80 at a concentration of about 0.01%.

43. The process of claim 34, wherein the buffering agent is at a concentration of about 5 mM to about 50 mM.

44. The process of claim 43, wherein the buffering agent is Tris at a concentration of about 20 mM.

45. The process of claim 34, wherein the electrolyte is at a concentration of about 5 mM to about 100 mM.

46. The process of claim 45, wherein the electrolyte is a sodium or potassium salt.

47. The process of claim 45, wherein the electrolyte is NaCl at a concentration of about 10 mM.

48. The process of claim 34, wherein prior to lyophilization, the pH is about 7.8 to about 8.2.

49. The process of claim 48, wherein the pH prior to lyophilization is about 8.0.

50. The process of claim 34, wherein lyophilization comprises
freezing the solution at a temperature of about −35° to about −50° C.;
initially drying the frozen solution at a primary drying pressure of about 20 to about 80 microns at a shelf-temperature of about −10° to about −40° C. for 24 to 78 hours; and
secondarily drying the freeze-dried product at a secondary drying pressure of about 20 to about 80 microns at a shelf temperature of about +10° to about +30° C. for 15 to 30 hours.

51. The process of claim 50, wherein freezing is carried out at about −45° C.; the initial freeze drying is at a primary drying pressure of about 60 microns and a shelf temperature of about −30° C. for 60 hours; and the secondary drying step is at a drying pressure about 60 microns and a shelf temperature of about +25° C. for about 24 hours.

52. The process of claim 34, wherein the process further comprises adding a bulking agent prior to lyophilization.

53. The process of claim 52, wherein the bulking agent is at a concentration of about 0.5 to about 1.5% by weight.

54. The process of claim 52, wherein the bulking agent is Dextran 40 at a concentration of about 0.9% by weight or hydroxyethyl starch 40 at a concentration of about 0.9% by weight.

55. The process of claim 34, wherein the conjugate has cytotoxic activity.

56. The process of claim 34, wherein the conjugate has anti-tumor activity.

57. The process of claim 34, wherein the conjugate is present in a therapeutically effective amount.

58. The process of claim 34, wherein the conjugate is N-acetyl gamma calicheamicin dimethyl hydrazide-4-(4-acetylephenoxy) butanoic acid (N-acetyl calicheamicin DMH-AcBut) covalently linked to G193, wherein the average loading is from about 5 to about 7 moles of N-acetyl calicheamicin DMH per mole of G193 and the low conjugated fraction (LCF) of the conjugate is less than about 10%.

59. A formulation comprising a calicheamicin-anti-Lewis Y antibody conjugate, a cryoprotectant, a surfactant, a buffering agent, and an electrolyte.

60. The formulation of claim 59, wherein the conjugate is at a concentration of about 0.5 mg/mL to about 2 mg/mL.

61. The formulation of claim 60, wherein the conjugate is at a concentration of about 1 mg/mL.

62. The formulation of claim 59, wherein the cryoprotectant is at a concentration of about 1.5% to about 6% by weight.

63. The formulation of claim 62, wherein the cryoprotectant is a sugar alcohol or a carbohydrate.

64. The formulation of claim 63, wherein the cryoprotectant is trehalose, mannitol, or sorbitol.

65. The formulation of claim 63, wherein the cryoprotectant is sucrose at a concentration of about 5%.

66. The formulation of claim 59, wherein the surfactant is at a concentration of about 0.005% to about 0.05%.

67. The formulation of claim 66, wherein the surfactant is Tween 80 at a concentration of about 0.01% by weight.

68. The formulation of claim 59, wherein the buffering agent is at a concentration of about 5 mM to about 50 mM.

69. The formulation of claim 69, wherein the buffering agent is Tris at a concentration of about 20 mM.

70. The formulation of claim 59, wherein the electrolyte is at a concentration of about 5 mM to about 100 mM.

71. The formulation of claim 70, wherein the electrolyte is a sodium or potassium salt.

72. The formulation of claim 71, wherein the electrolyte is NaCl at a concentration of about 10 mM.

73. The formulation of claim 59, wherein the pH is about 7.8 to about 8.2.

74. The formulation of claim 73, wherein the pH is about 8.0.

75. The formulation of claim 59, wherein the calicheamicin is an N-acyl derivative of calicheamicin or a disulfide analog of calicheamicin.

76. The formulation of claim 75, wherein the calicheamicin is N-acetyl gamma calicheamicin dimethyl hydrazide (N-acetyl calicheamicin DMH).

77. The formulation of claim 59, wherein the hydrolyzable linker is 4-(4-acetylephenoxy) butanoic acid (AcBut).

78. The formulation of claim 59, wherein the anti-Lewis Y antibody is G193 or Hu3S193.

79. The formulation of claim 59, wherein the average loading is from about 5 to about 7 moles of calicheamicin per mole of anti-Lewis Y antibody.

80. The formulation of claim 59, wherein the conjugate has a K_{D} of about 1×10⁻⁷ M to about 4×10⁻⁷ M.

81. The formulation of claim 59, wherein the conjugate has a K_{D} of about 3.4×10⁻⁷ M.

82. The formulation of claim 59, wherein the conjugate binds the Lewis Y antigen and does not bind Lewis X and H-2 blood group antigens.

83. The formulation of claim 59, wherein the conjugate has cytotoxic activity.

84. The formulation of claim 59, wherein the conjugate has anti-tumor activity.

85. The formulation of claim 59, wherein the conjugate is present in a therapeutically effective amount.

86. The formulation of claim 59, wherein the conjugate is N-acetyl gamma calicheamicin dimethyl hydrazide-4-(4-acetylephenoxy) butanoic acid (N-acetyl calicheamicin DMH-AcBut) covalently linked to G193, wherein the average loading is from about 5 to about 7 moles of N-acetyl calicheamicin DMH per mole of G193 and the low conjugated fraction (LCF) of the conjugate is less than about 10%.

87. The formulation of claim 86, wherein the conjugate is at a concentration of 1 mg/mL, the cryoprotectant is sucrose at a concentration of about 5%, the surfactant is Tween 80 at a concentration of about 0.01% by weight, the buffering agent is Tris at a concentration of about 20 mM, and the electrolyte is NaCl at a concentration of about 10 mM, wherein the pH is about 8.0.

88. A method of treating cancer comprising administering a therapeutically effective amount of a composition of claim 21.

89. The method of claim 88, wherein the cancer is positive for Lewis Y antigen.

90. The method of claim 89, wherein the cancer is a carcinoma.

91. The method of claim 88, wherein the composition is administered as a second-line monotherapy.

92. The method of claim 88, wherein the composition is administered as a first-line combination therapy.

93. The method of claim 88, wherein the cancer is Non-Small Cell Lung Cancer (NSCLC) or breast cancer.

94. The method of claim 88, wherein the cancer is prostate cancer or colorectal cancer.

95. The method of claim 88, wherein the composition is administered in combination with a bioactive agent.

96. The method of claim 95, wherein the bioactive agent is an anti-cancer agent.

97. A method of treating NSCLC comprising administering a composition of claim 33.

98. A method of treating breast cancer comprising administering a composition of claim 33.

99. A method of treating a patient with a proliferative disorder, the method comprising administering a therapeutically effective amount of the composition of claim 21.

100. A kit comprising (i) a container which holds the formulation of claim 59; and (ii) instructions for reconstituting the formulation with a diluent to a conjugate concentration in the reconstituted formulation within the range from about 0.5 mg/mL to about 5 mg/mL.
